(19) United States
(12) Patent Application Publication
(10) Pub. No.: US 2017/0152226 A1
(43) Pub. Date: Jun. 1, 2017

(54) FUNCTIONALISED AND SUBSTITUTED INDOLES AS ANTI-CANCER AGENTS

(71) Applicant: Novogen Ltd., Hornsby, New South Wales (AU)

(72) Inventors: Andrew Heaton, Hornsby (AU); Eleanor Eiffe, Hornsby (AU); Narender Pottabathini, Nacharam (IN); Peter Gunning, Sydney (AU)

(21) Appl. No.: 15/323,694

(22) PCT Filed: Jul. 16, 2015
(86) PCT No.: PCT/AU2015/050400

§ 371 Date: Jan. 3, 2017

Related U.S. Application Data
(60) Provisional application No. 62/025,207, filed on Jul. 16, 2014.

(51) Int. Cl. C07D 209/08; C07D 405/12; C07D 401/12

(52) U.S. Cl. CPC C07D 209/08; C07D 401/12; C07D 405/12

(57) ABSTRACT

The present invention relates to anti-tropomyosin compounds, processes for their preparation, and methods for treating or preventing a disease or disorder, such as a proliferative disease (preferably cancer), using compounds of the invention.

### FIELD OF THE INVENTION

The present invention relates broadly to pharmaceutical agents as treatments for proliferative disease such as cancer and a range of degenerative diseases such as osteoarthritis, atherosclerosis, heart disease and inflammatory bowel disease. In particular, the present invention relates to pharmaceutical agents which comprise aryl and/or alkyl substituted indole compounds. The invention further relates to methods for treating or preventing a disease or disorder, such as a proliferative disorder (preferably cancer). The invention also relates to processes for preparing the compounds.

### BACKGROUND OF THE INVENTION

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art. Cancer kills many thousands of people and is the second largest cause of death in the USA. There have been significant breakthroughs made in treating or preventing a wide variety of cancers. For example patients with breast cancer have benefited from early screening programs as well as a variety of surgical techniques. However, these often prove physically and emotionally debilitating. Moreover, patients who have undergone surgery and subsequent chemotherapy often experience a recurrence in their disease.

A potential new method of specifically attacking cancer cells is through disruption of cancer cells' cellular skeletal system comprised predominantly of actin. The actin cytoskeleton is intimately involved in cell division and cell migration. However, actin plays a ubiquitous role as the cytoskeleton of tumour cells and the actin filaments of the muscle sarcomere. The differing roles but similarity in structure make actin a hard target for drug development, due to unwanted off-target side effects.

### SUMMARY OF THE INVENTION

The invention seeks to address one or more of the above mentioned problems, and/or to provide improvements in therapy (e.g. cancer therapy) and in one embodiment provides an anti-tropomyosin compound.

In a first aspect of the invention there is provided a compound of general formula (I), or a pharmaceutically acceptable drug or prodrug thereof:

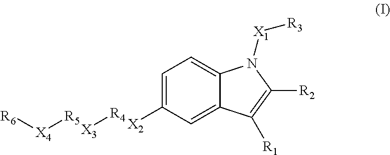

wherein:
R₁ and R₂ are independently H or C₁-C₆ alkyl;
R₃ is N(R₇)₂ or a 3- to 7-membered carbocyclic ring wherein between 1 and 3 ring carbon atoms may optionally be replaced by S, N, O, NH or NR₇ and wherein the ring may optionally be substituted by R₇;
R₄ and R₅ are independently

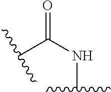 [SMILES: *NC(*)=O]

or a 5- or 6-membered carbocyclic ring wherein between 1 and 3 ring carbon atoms may optionally be replaced with S, N, O, NH or NR₈ and wherein the ring may optionally be substituted by R₈;
R₆ is a C₁-C₆ alkyl group, a C₂-C₆ alkene group or a monocyclic or bicyclic carbocyclic ring having between 5 and 10 ring carbon atoms wherein 1 or 2 ring carbon atoms may optionally be replaced with S, O, N, NH or NR₇ and wherein the ring may optionally be substituted with R₈, or R₆ is

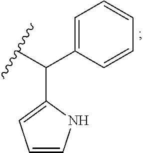 [SMILES: *C(c1ccccc1)c1ccc[nH]1]

X₁ is absent or is an alkyl group having between 1 and 10 carbon atoms, or an alkenyl group having between 2 and 10 carbon atoms;
X₂, X₃ and X₄ are independently absent or selected from the group consisting of: S, O, NH, NHR₇, C(O), C(O)NH, an alkyl group having between 1 and 10 carbon atoms, an alkene group having between 2 and 10 carbon atoms, CH(R₇)CHC(R₇)C(O), (CH₂)₀₋₅C(R₇)C(R₇)(CH₂)₀₋₅, and a 5- or 6-membered carbocyclic ring wherein between 1 and 3 ring carbon atoms may optionally be replaced by S, N, O, NH or NR₇;
X₅ is O, NH, NR₇ or S;
R₇ is H, C₁-C₆ alkyl, (CH₂)₁₋₅OMe, CF₃, CN or OCF₃; and
R₈ is H, OH, alkyl (e.g. C₁-C₆ alkyl), alkenyl (e.g. C₂-C₆ alkenyl), halo, alkoxy, amino, alkylamino, dialkylamino or a dioxolane ring fused to 2 adjacent carbon atoms of R₄, R₅ or R₆.

X₁ may be an alkyl group having between 1 and 10 carbon atoms (e.g. between 1 and 5 carbon atoms).

R₃ may be N(R₇)₂ or a 4-, 5-, 6- or 7-membered carbocyclic ring (e.g. cycloalkyl) wherein between 1 and 3 ring carbon atoms may optionally be replaced by S, N, O, NH or NR₇ and wherein the ring may optionally be substituted by R₇.

R₁ and R₂ may be independently C₁-C₆ alkyl (e.g. R₁ may be, for example, CH₃ or CH₂CH₃ and R₂ may be, for example, CH₃ or CH₂CH₃).

X₂, X₃ and X₄ may be independently selected from the group consisting of: S, O, NH, NHR₇, C(O), C(O)NH, an alkyl group having between 1 and 10 carbon atoms (e.g. between 1 and 5 carbon atoms), CH(R₇)CHC(R₇)C(O), (CH₂)₀₋₅C(R₇)C(R₇)(CH₂)₀₋₅, and a 5-membered carbocyclic ring (e.g. aryl) wherein between 1 and 3 ring carbon atoms (e.g. 1 or 2 ring carbon atoms) may optionally be replaced by S, N, O, NH or NR₇ (e.g. N and/or O).

R₄ and R₅ may be independently a 5- or 6-membered aryl or cycloalkyl group wherein between 1 and 3 ring carbon atoms may optionally be replaced with S, N, O, NH or NR₈ and wherein the ring may optionally be substituted by R₈.

R₆ may be a C₁-C₆ alkyl group (e.g. CH₃ or CH₂CH₃) or a monocyclic or bicyclic aryl group having between 6 and 10 ring carbon atoms wherein 1 or 2 ring carbon atoms may optionally be replaced with S, O, N, NH or NR₇ and wherein the ring may optionally be substituted with R₈. R₆ may be:

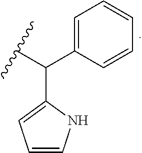 [SMILES: *C(c1ccccc1)c1ccc[nH]1]

In one embodiment, the compound of formula (I), or a pharmaceutically acceptable drug or prodrug thereof, is:

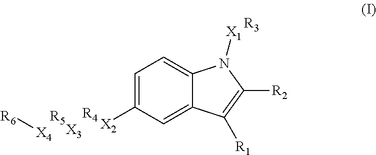

wherein:

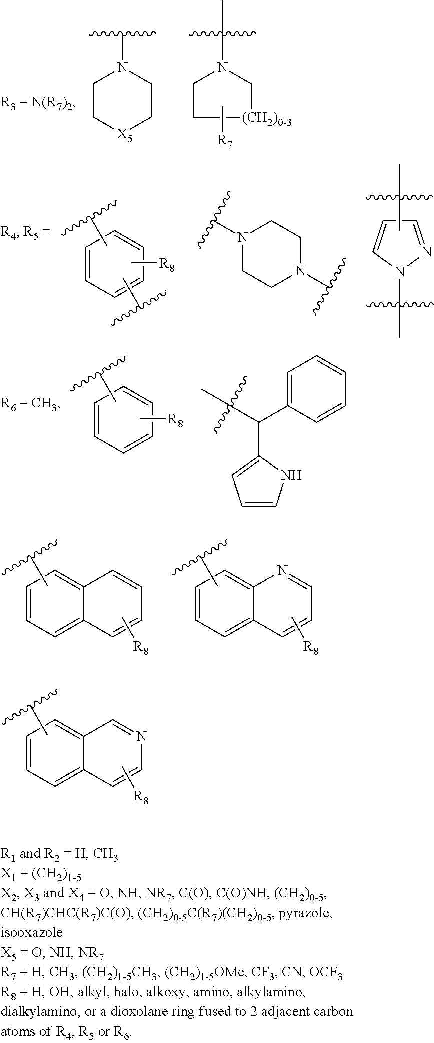

R₁ and R₂ may both be CH₃ or CH₂CH₃.

X₁ may be an alkyl group having between 1 and 5 carbon atoms (e.g. CH₂, (CH₂)₂ or (CH₂)₃).

R₃ may be a 4-, 5-, 6- or 7-membered cycloalkyl group wherein between 1 and 3 ring carbon atoms may optionally be replaced by S, N, O, NH or NR₇ and wherein the ring may optionally be substituted by R₇, such as:

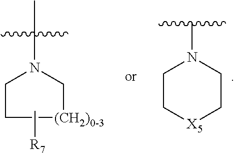

R₃ may be a 6-membered cycloalkyl group wherein between 1 and 3 ring carbon atoms may optionally be replaced by S, N, O, NH or NR₇ and wherein the ring may optionally be substituted by R₇, such as:

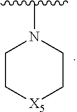

X₅ may be NH or NR₇. R₇ may be C₁-C₆ alkyl (e.g. CH₃ or CH₂CH₃).

X₂ may be an alkyl group having between 1 and 10 carbon atoms, O or NH. X₂ may be (CH₂)₁₋₅ (e.g. CH₂, (CH₂)₂ or (CH₂)₃).

R₄ may be a 5- or 6-membered aryl group wherein between 1 and 3 ring carbon atoms may optionally be replaced with S, N, O, NH or NR₈ and wherein the ring may optionally be substituted by R₈, such as:

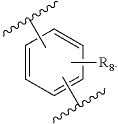

R₈ may be H.

X₃ may be C(O).

R₅ may be a 5- or 6-membered cycloalkyl group wherein between 1 and 3 ring carbon atoms may optionally be replaced with S, N, O, NH or NR₈ and wherein the ring may optionally be substituted by R₈, such as:

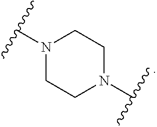 [SMILES: *N1CCN(*)CC1]

X₄ may be an alkyl group having between 1 and 5 carbon atoms (e.g. CH₂, (CH₂)₂ or (CH₂)₃).

R₆ may be a bicyclic aryl group having 9 or 10 ring carbon atoms wherein 1 or 2 ring carbon atoms may optionally be replaced with S, O, N, NH or NR₇ and wherein the ring may optionally be substituted with R₈. R₆ may be selected from:

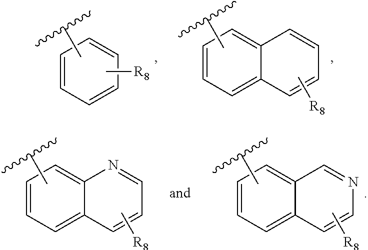

R₈ may be selected from H, alkoxy, halo and a dioxalane ring fused to two adjacent carbon atoms of R₆. R₈ may be alkoxy (e.g. OCH₃ or OCH₂CH₃). R₈ may be halo (e.g. fluorine).

Preferably, the compounds of the first aspect of the invention are exemplified in the following structures:

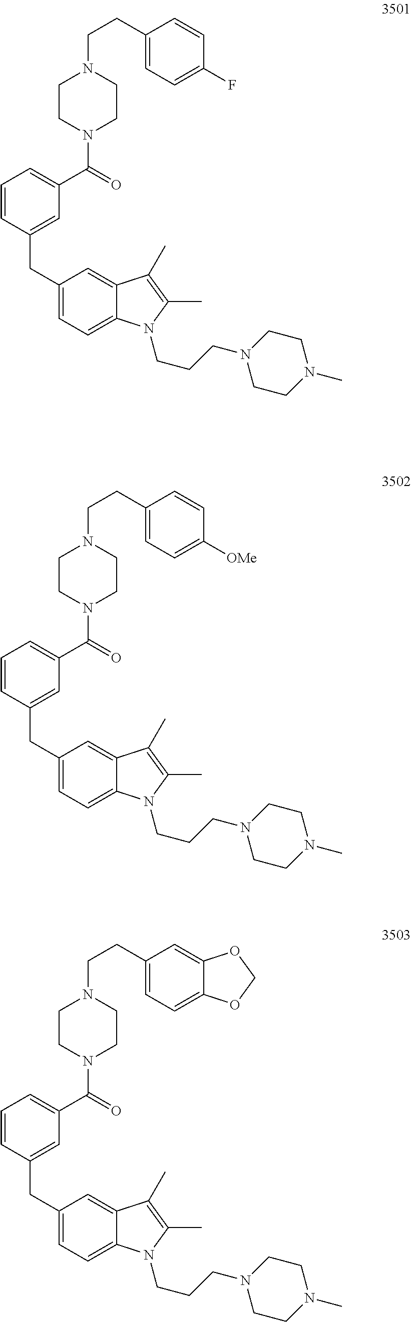 [SMILES: Cc1c(C)n(CCCN2CCN(C)CC2)c2ccc(Cc3cccc(C(=O)N4CCN(CCc5ccc(F)cc5)CC4)c3)cc12 ; COc1ccc(CCN2CCN(C(=O)c3cccc(Cc4ccc5c(c4)c(C)c(C)n5CCCN4CCN(C)CC4)c3)CC2)cc1 ; Cc1c(C)n(CCCN2CCN(C)CC2)c2ccc(Cc3cccc(C(=O)N4CCN(CCc5ccc6c(c5)OCO6)CC4)c3)cc12]

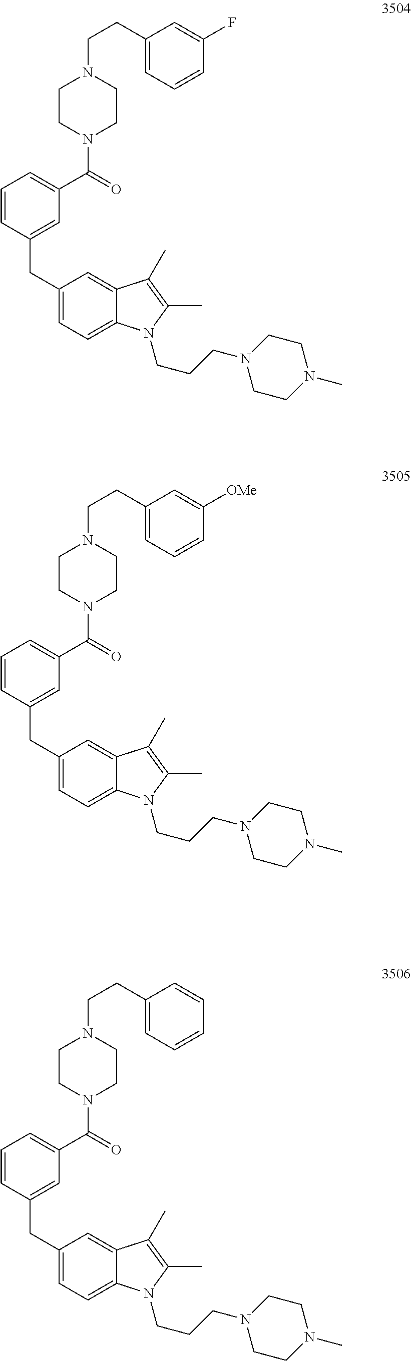 [SMILES: Cc1c(C)n(CCCN2CCN(C)CC2)c2ccc(Cc3cccc(C(=O)N4CCN(CCc5cccc(F)c5)CC4)c3)cc12 ; COc1cccc(CCN2CCN(C(=O)c3cccc(Cc4ccc5c(c4)c(C)c(C)n5CCCN4CCN(C)CC4)c3)CC2)c1 ; Cc1c(C)n(CCCN2CCN(C)CC2)c2ccc(Cc3cccc(C(=O)N4CCN(CCc5ccccc5)CC4)c3)cc12]

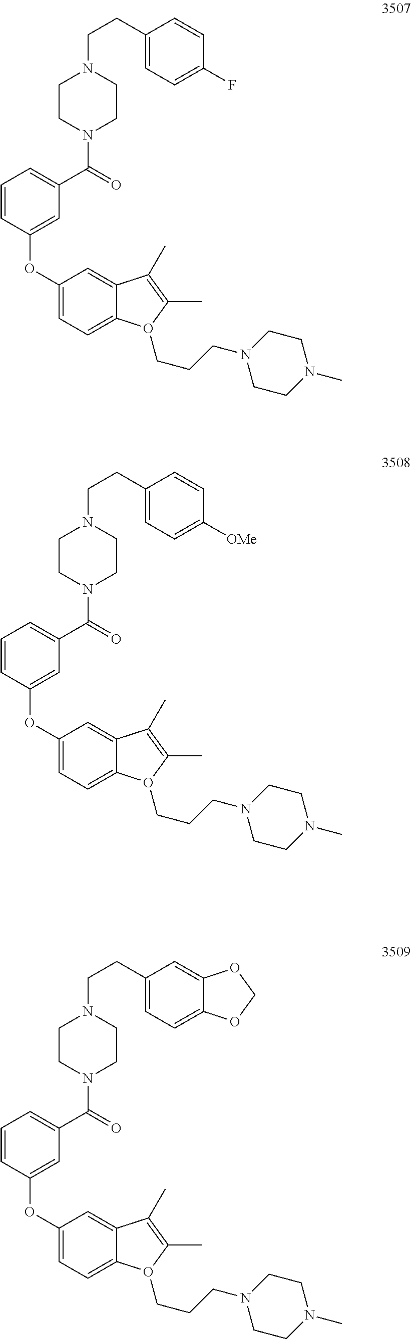

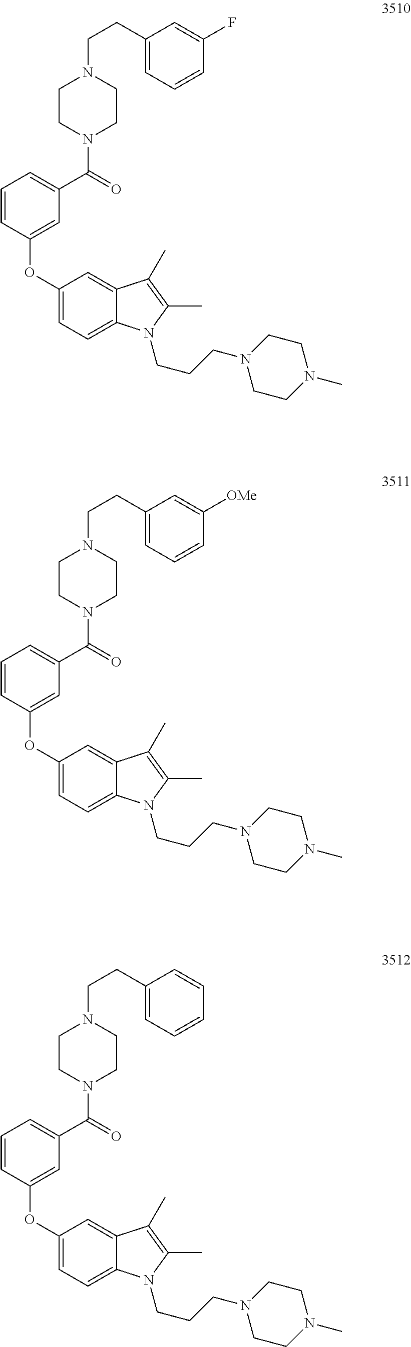 [SMILES: Cc1c(C)n(CCCN2CCN(C)CC2)c2ccc(Oc3cccc(C(=O)N4CCN(CCc5cccc(F)c5)CC4)c3)cc12 ; COc1cccc(CCN2CCN(C(=O)c3cccc(Oc4ccc5c(c4)c(C)c(C)n5CCCN4CCN(C)CC4)c3)CC2)c1 ; Cc1c(C)n(CCCN2CCN(C)CC2)c2ccc(Oc3cccc(C(=O)N4CCN(CCc5ccccc5)CC4)c3)cc12]

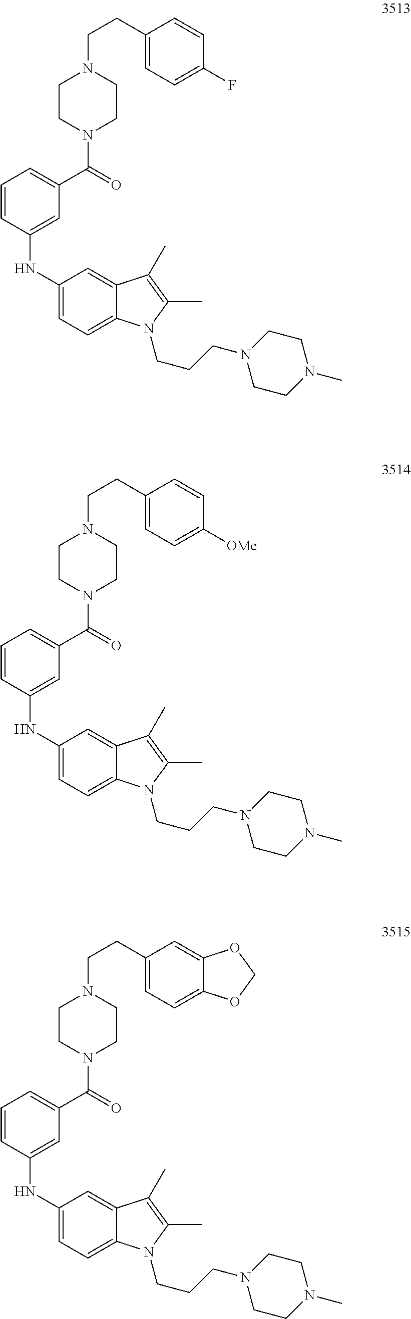 [SMILES: Cc1c(C)n(CCCN2CCN(C)CC2)c2ccc(Nc3cccc(C(=O)N4CCN(CCc5ccc(F)cc5)CC4)c3)cc12 ; COc1ccc(CCN2CCN(C(=O)c3cccc(Nc4ccc5c(c4)c(C)c(C)n5CCCN4CCN(C)CC4)c3)CC2)cc1 ; Cc1c(C)n(CCCN2CCN(C)CC2)c2ccc(Nc3cccc(C(=O)N4CCN(CCc5ccc6c(c5)OCO6)CC4)c3)cc12]

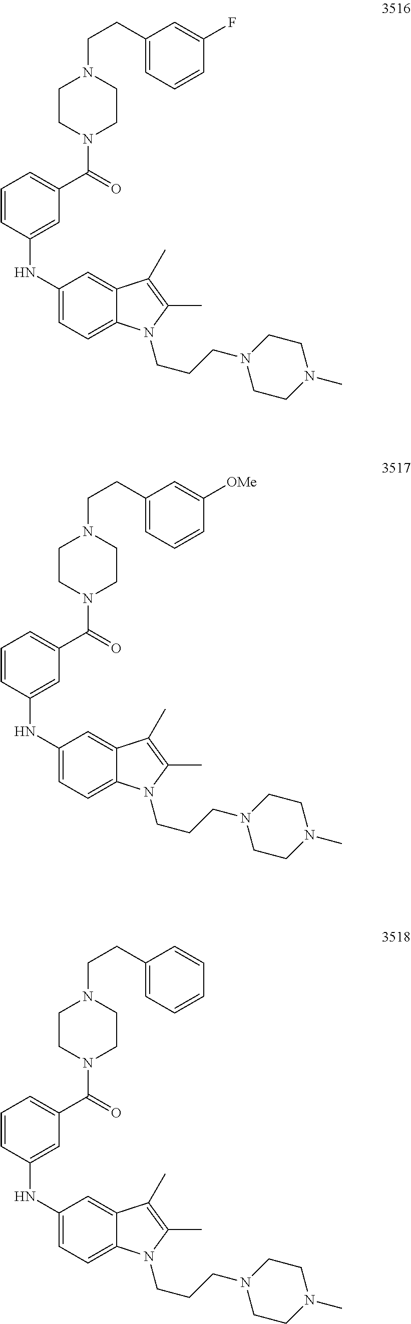 [SMILES: Cc1c(C)n(CCCN2CCN(C)CC2)c2ccc(Nc3cccc(C(=O)N4CCN(CCc5cccc(F)c5)CC4)c3)cc12 ; COc1cccc(CCN2CCN(C(=O)c3cccc(Nc4ccc5c(c4)c(C)c(C)n5CCCN4CCN(C)CC4)c3)CC2)c1 ; Cc1c(C)n(CCCN2CCN(C)CC2)c2ccc(Nc3cccc(C(=O)N4CCN(CCc5ccccc5)CC4)c3)cc12]

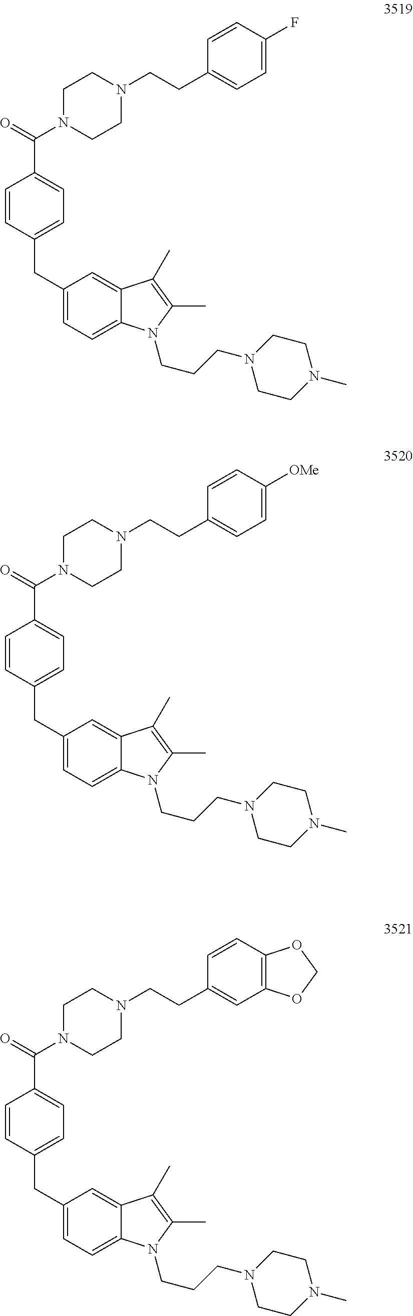 [SMILES: Cc1c(C)n(CCCN2CCN(C)CC2)c2ccc(Cc3ccc(C(=O)N4CCN(CCc5ccc(F)cc5)CC4)cc3)cc12 ; COc1ccc(CCN2CCN(C(=O)c3ccc(Cc4ccc5c(c4)c(C)c(C)n5CCCN4CCN(C)CC4)cc3)CC2)cc1 ; Cc1c(C)n(CCCN2CCN(C)CC2)c2ccc(Cc3ccc(C(=O)N4CCN(CCc5ccc6c(c5)OCO6)CC4)cc3)cc12]

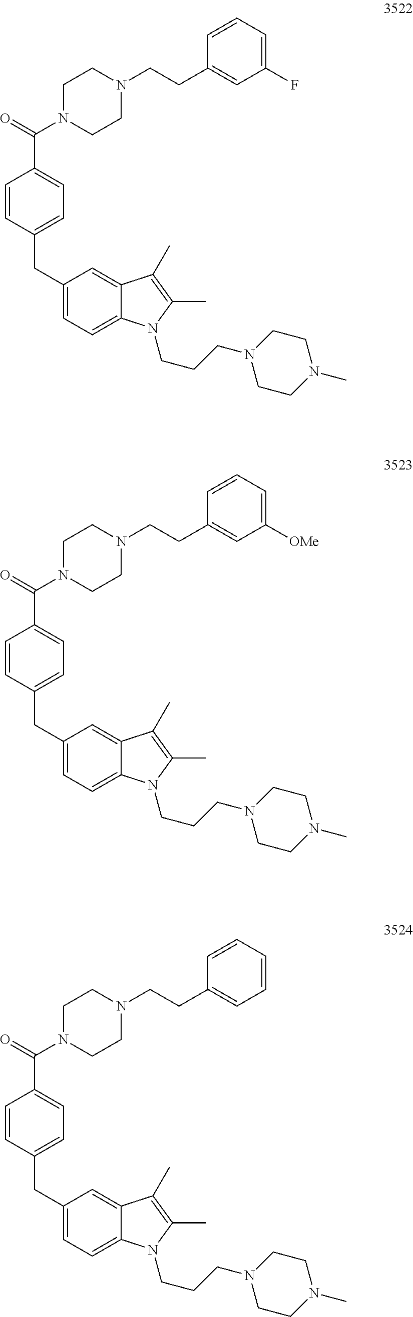 [SMILES: Cc1c(C)n(CCCN2CCN(C)CC2)c2ccc(Cc3ccc(C(=O)N4CCN(CCc5cccc(F)c5)CC4)cc3)cc12 ; COc1cccc(CCN2CCN(C(=O)c3ccc(Cc4ccc5c(c4)c(C)c(C)n5CCCN4CCN(C)CC4)cc3)CC2)c1 ; Cc1c(C)n(CCCN2CCN(C)CC2)c2ccc(Cc3ccc(C(=O)N4CCN(CCc5ccccc5)CC4)cc3)cc12]

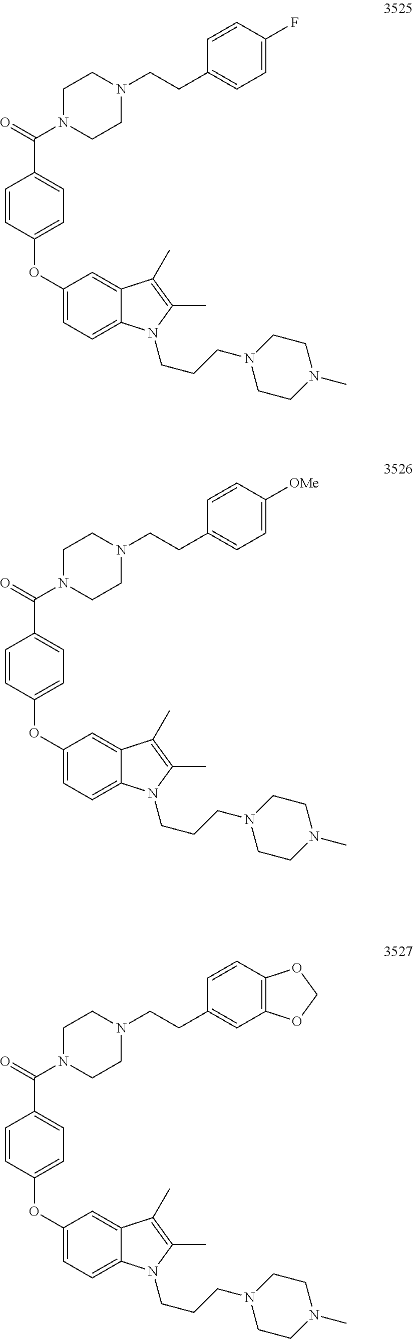 [SMILES: Cc1c(C)n(CCCN2CCN(C)CC2)c2ccc(Oc3ccc(C(=O)N4CCN(CCc5ccc(F)cc5)CC4)cc3)cc12 ; COc1ccc(CCN2CCN(C(=O)c3ccc(Oc4ccc5c(c4)c(C)c(C)n5CCCN4CCN(C)CC4)cc3)CC2)cc1 ; Cc1c(C)n(CCCN2CCN(C)CC2)c2ccc(Oc3ccc(C(=O)N4CCN(CCc5ccc6c(c5)OCO6)CC4)cc3)cc12]

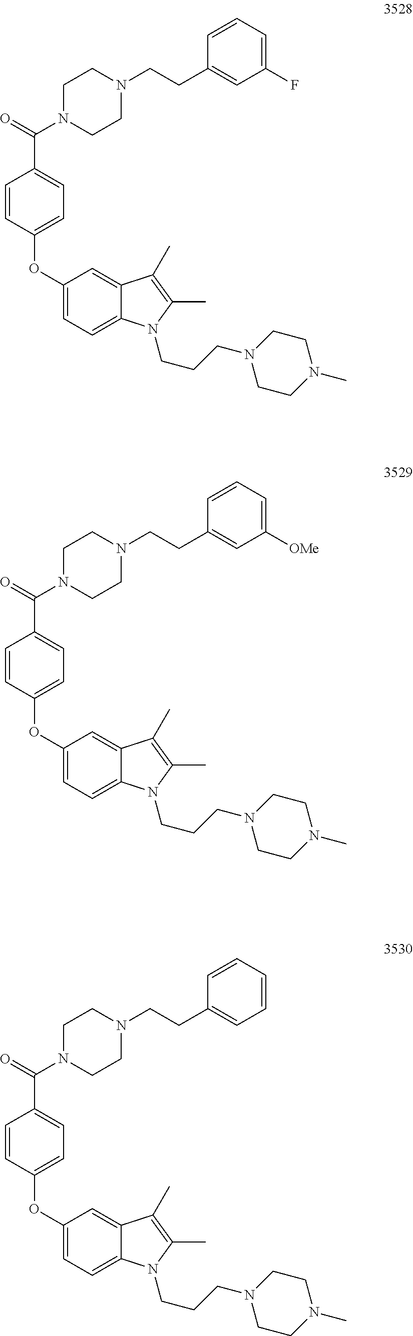 [SMILES: Cc1c(C)n(CCCN2CCN(C)CC2)c2ccc(Oc3ccc(C(=O)N4CCN(CCc5cccc(F)c5)CC4)cc3)cc12 ; COc1cccc(CCN2CCN(C(=O)c3ccc(Oc4ccc5c(c4)c(C)c(C)n5CCCN4CCN(C)CC4)cc3)CC2)c1 ; Cc1c(C)n(CCCN2CCN(C)CC2)c2ccc(Oc3ccc(C(=O)N4CCN(CCc5ccccc5)CC4)cc3)cc12]

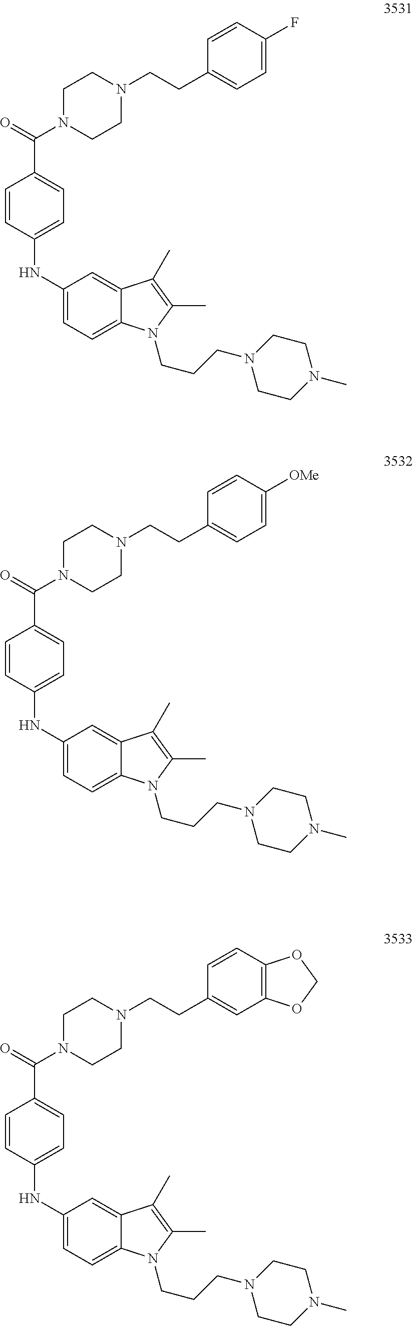 [SMILES: Cc1c(C)n(CCCN2CCN(C)CC2)c2ccc(Nc3ccc(C(=O)N4CCN(CCc5ccc(F)cc5)CC4)cc3)cc12 ; COc1ccc(CCN2CCN(C(=O)c3ccc(Nc4ccc5c(c4)c(C)c(C)n5CCCN4CCN(C)CC4)cc3)CC2)cc1 ; Cc1c(C)n(CCCN2CCN(C)CC2)c2ccc(Nc3ccc(C(=O)N4CCN(CCc5ccc6c(c5)OCO6)CC4)cc3)cc12]

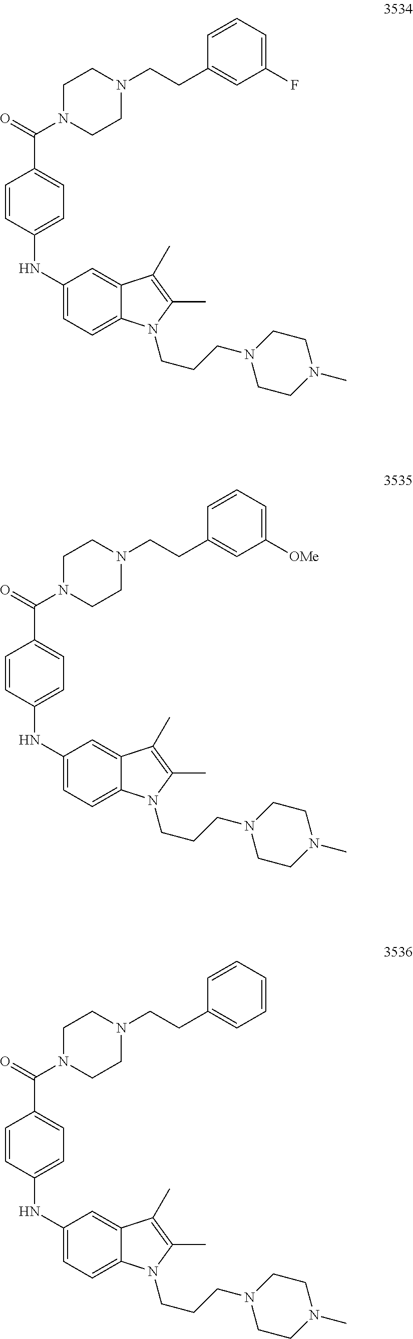 [SMILES: Cc1c(C)n(CCCN2CCN(C)CC2)c2ccc(Nc3ccc(C(=O)N4CCN(CCc5cccc(F)c5)CC4)cc3)cc12 ; COc1cccc(CCN2CCN(C(=O)c3ccc(Nc4ccc5c(c4)c(C)c(C)n5CCCN4CCN(C)CC4)cc3)CC2)c1 ; Cc1c(C)n(CCCN2CCN(C)CC2)c2ccc(Nc3ccc(C(=O)N4CCN(CCc5ccccc5)CC4)cc3)cc12]

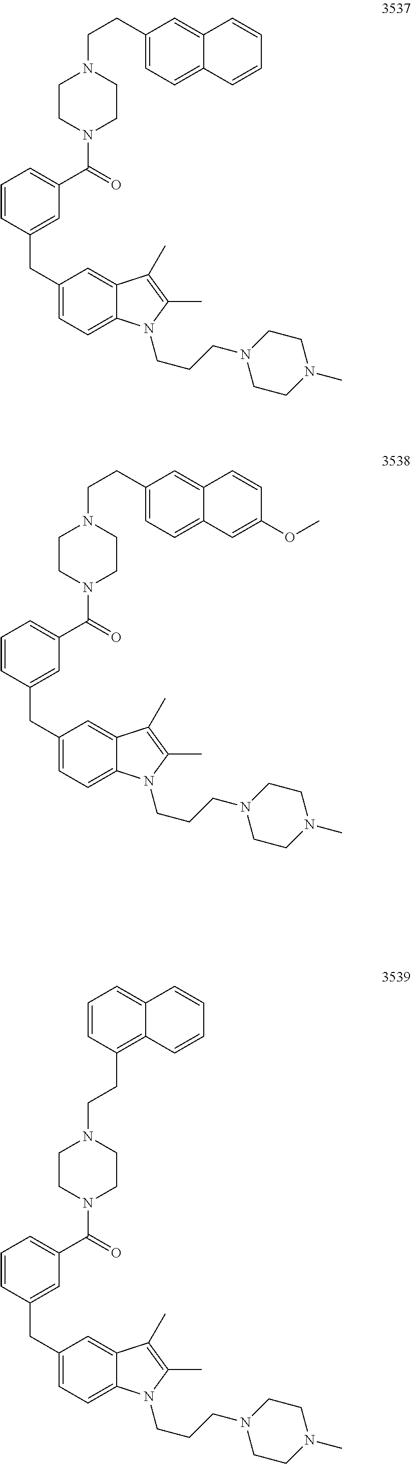 [SMILES: Cc1c(C)n(CCCN2CCN(C)CC2)c2ccc(Cc3cccc(C(=O)N4CCN(CCc5ccc6ccccc6c5)CC4)c3)cc12 ; COc1ccc2cc(CCN3CCN(C(=O)c4cccc(Cc5ccc6c(c5)c(C)c(C)n6CCCN5CCN(C)CC5)c4)CC3)ccc2c1 ; Cc1c(C)n(CCCN2CCN(C)CC2)c2ccc(Cc3cccc(C(=O)N4CCN(CCc5cccc6ccccc56)CC4)c3)cc12]

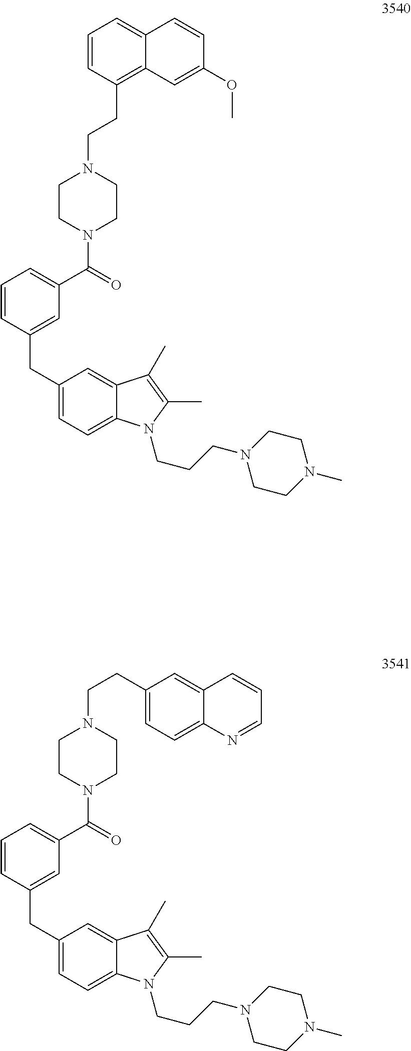 [SMILES: COc1ccc2cccc(CCN3CCN(C(=O)c4cccc(Cc5ccc6c(c5)c(C)c(C)n6CCCN5CCN(C)CC5)c4)CC3)c2c1 ; Cc1c(C)n(CCCN2CCN(C)CC2)c2ccc(Cc3cccc(C(=O)N4CCN(CCc5ccc6ncccc6c5)CC4)c3)cc12]

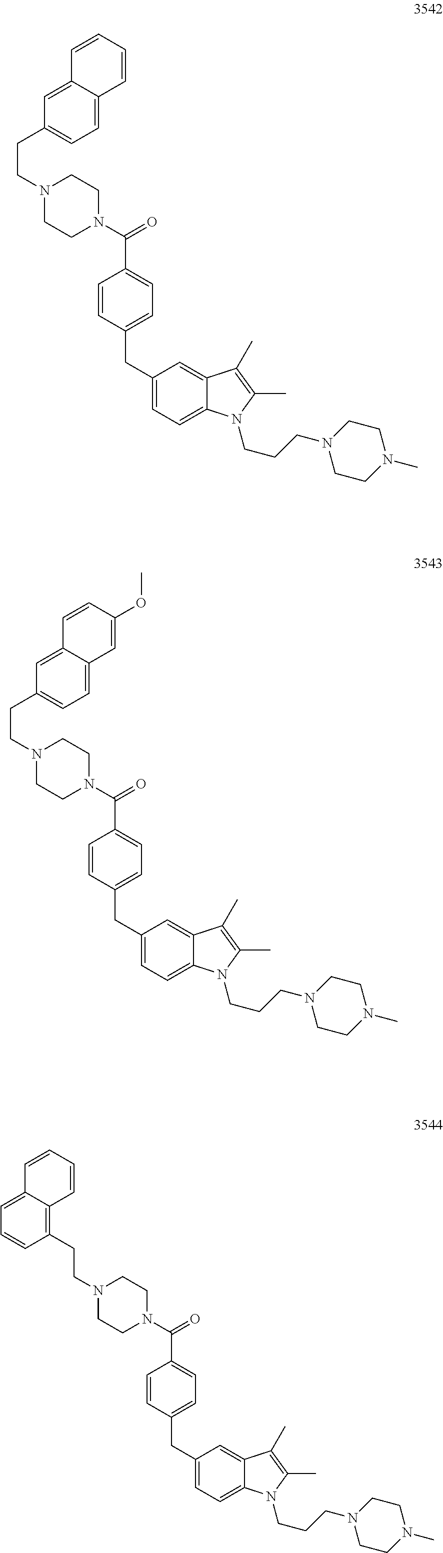 [SMILES: Cc1c(C)n(CCCN2CCN(C)CC2)c2ccc(Cc3ccc(C(=O)N4CCN(CCc5ccc6ccccc6c5)CC4)cc3)cc12 ; COc1ccc2cc(CCN3CCN(C(=O)c4ccc(Cc5ccc6c(c5)c(C)c(C)n6CCCN5CCN(C)CC5)cc4)CC3)ccc2c1 ; Cc1c(C)n(CCCN2CCN(C)CC2)c2ccc(Cc3ccc(C(=O)N4CCN(CCc5cccc6ccccc56)CC4)cc3)cc12]

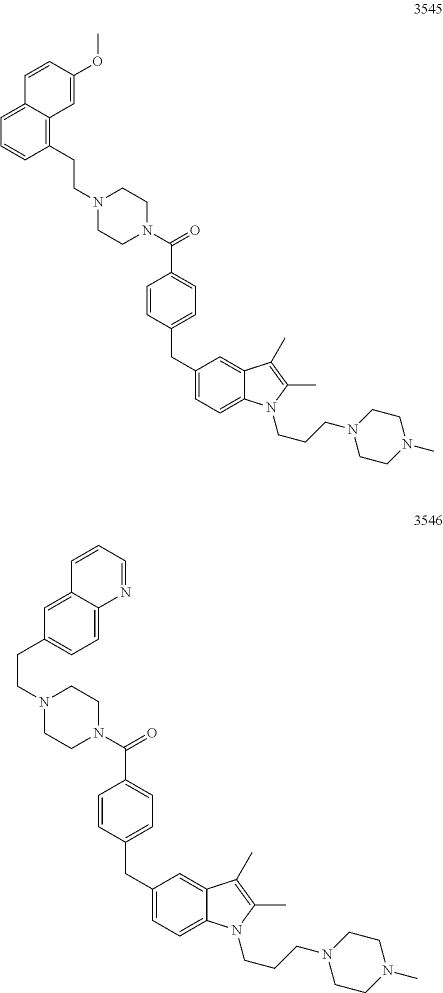 [SMILES: COc1ccc2cccc(CCN3CCN(C(=O)c4ccc(Cc5ccc6c(c5)c(C)c(C)n6CCCN5CCN(C)CC5)cc4)CC3)c2c1 ; Cc1c(C)n(CCCN2CCN(C)CC2)c2ccc(Cc3ccc(C(=O)N4CCN(CCc5ccc6ncccc6c5)CC4)cc3)cc12]

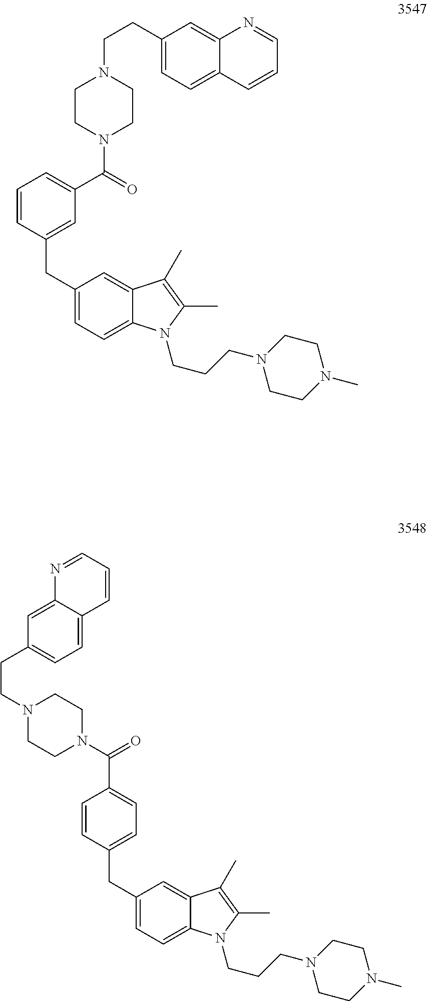 [SMILES: Cc1c(C)n(CCCN2CCN(C)CC2)c2ccc(Cc3cccc(C(=O)N4CCN(CCc5ccc6cccnc6c5)CC4)c3)cc12 ; Cc1c(C)n(CCCN2CCN(C)CC2)c2ccc(Cc3ccc(C(=O)N4CCN(CCc5ccc6cccnc6c5)CC4)cc3)cc12]

In one embodiment, the compounds are:
(3-((2,3-dimethyl-1-(3-(4-methylpiperazin-1-yl)propyl)-1H-indol-5-yl)methyl)phenyl)(4-(4-fluorophenethyl)piperazin-1-yl)methanone
(3-((2,3-dimethyl-1-(3-(4-methylpiperazin-1-yl)propyl)-1H-indol-5-yl)methyl)phenyl)(4-(4-methoxyphenethyl)piperazin-1-yl)methanone
(4-(2-(benzo[d][1,3]dioxol-5-yl)ethyl)piperazin-1-yl)(3-((2,3-dimethyl-1-(3-(4-methylpiperazin-1-yl)propyl)-1H-indol-5-yl)methyl)phenyl)methanone
(3-((2,3-dimethyl-1-(3-(4-methylpiperazin-1-yl)propyl)-1H-indol-5-yl)methyl)phenyl)(4-(3-fluorophenethyl)piperazin-1-yl)methanone
(3-((2,3-dimethyl-1-(3-(4-methylpiperazin-1-yl)propyl)-1H-indol-5-yl)methyl)phenyl)(4-(3-methoxyphenethyl)piperazin-1-yl)methanone
(3-((2,3-dimethyl-1-(3-(4-methylpiperazin-1-yl)propyl)-1H-indol-5-yl)methyl)phenyl)(4-phenethylpiperazin-1-yl)methanone
(3-((2,3-dimethyl-1-(3-(4-methylpiperazin-1-yl)propyl)-1H-indol-5-yl)oxy)phenyl)(4-(4-fluorophenethyl)piperazin-1-yl)methanone
(3-((2,3-dimethyl-1-(3-(4-methylpiperazin-1-yl)propyl)-1H-indol-5-yl)oxy)phenyl)(4-(4-methoxyphenethyl)piperazin-1-yl)methanone
(4-(2-(benzo[d][1,3]dioxol-5-yl)ethyl)piperazin-1-yl)(3-((2,3-dimethyl-1-(3-(4-methylpiperazin-1-yl)propyl)-1H-indol-5-yl)oxy)phenyl)methanone
(3-((2,3-dimethyl-1-(3-(4-methylpiperazin-1-yl)propyl)-1H-indol-5-yl)oxy)phenyl)(4-(3-fluorophenethyl)piperazin-1-yl)methanone
(3-((2,3-dimethyl-1-(3-(4-methylpiperazin-1-yl)propyl)-1H-indol-5-yl)oxy)phenyl)(4-(3-methoxyphenethyl)piperazin-1-yl)methanone
(3-((2,3-dimethyl-1-(3-(4-methylpiperazin-1-yl)propyl)-1H-indol-5-yl)oxy)phenyl)(4-phenethylpiperazin-1-yl)methanone
(3-((2,3-dimethyl-1-(3-(4-methylpiperazin-1-yl)propyl)-1H-indol-5-yl)amino)phenyl)(4-(4-fluorophenethyl)piperazin-1-yl)methanone
(3-((2,3-dimethyl-1-(3-(4-methylpiperazin-1-yl)propyl)-1H-indol-5-yl)amino)phenyl)(4-(4-methoxyphenethyl)piperazin-1-yl)methanone
(4-(2-(benzo[d][1,3]dioxol-5-yl)ethyl)piperazin-1-yl)(3-((2,3-dimethyl-1-(3-(4-methylpiperazin-1-yl)propyl)-1H-indol-5-yl)amino)phenyl)methanone
(3-((2,3-dimethyl-1-(3-(4-methylpiperazin-1-yl)propyl)-1H-indol-5-yl)amino)phenyl)(4-(3-fluorophenethyl)piperazin-1-yl)methanone
(3-((2,3-dimethyl-1-(3-(4-methylpiperazin-1-yl)propyl)-1H-indol-5-yl)amino)phenyl)(4-(3-methoxyphenethyl)piperazin-1-yl)methanone
(3-((2,3-dimethyl-1-(3-(4-methylpiperazin-1-yl)propyl)-1H-indol-5-yl)amino)phenyl)(4-phenethylpiperazin-1-yl)methanone
(4-((2,3-dimethyl-1-(3-(4-methylpiperazin-1-yl)propyl)-1H-indol-5-yl)methyl)phenyl) (4-(4-fluorophenethyl)piperazin-1-yl)methanone
(4-((2,3-dimethyl-1-(3-(4-methylpiperazin-1-yl)propyl)-1H-indol-5-yl)methyl)phenyl)(4-(4-methoxyphenethyl)piperazin-1-yl)methanone
(4-(2-(benzo[d][1,3]dioxol-5-yl)ethyl)piperazin-1-yl)(4-((2,3-dimethyl-1-(3-(4-methylpiperazin-1-yl)propyl)-1H-indol-5-yl)methyl)phenyl)methanone
(4-((2,3-dimethyl-1-(3-(4-methylpiperazin-1-yl)propyl)-1H-indol-5-yl)methyl)phenyl)(4-(3-fluorophenethyl)piperazin-1-yl)methanone
(4-((2,3-dimethyl-1-(3-(4-methylpiperazin-1-yl)propyl)-1H-indol-5-yl)methyl)phenyl)(4-(3-methoxyphenethyl)piperazin-1-yl)methanone
(4-((2,3-dimethyl-1-(3-(4-methylpiperazin-1-yl)propyl)-1H-indol-5-yl)methyl)phenyl)(4-phenethylpiperazin-1-yl)methanone
(4-((2,3-dimethyl-1-(3-(4-methylpiperazin-1-yl)propyl)-1H-indol-5-yl)oxy)phenyl)(4-(4-fluorophenethyl)piperazin-1-yl)methanone
(4-((2,3-dimethyl-1-(3-(4-methylpiperazin-1-yl)propyl)-1H-indol-5-yl)oxy)phenyl)(4-(4-methoxyphenethyl)piperazin-1-yl)methanone
(4-(2-(benzo[d][1,3]dioxol-5-yl)ethyl)piperazin-1-yl)(4-((2,3-dimethyl-1-(3-(4-methylpiperazin-1-yl)propyl)-1H-indol-5-yl)oxy)phenyl)methanone
(4-((2,3-dimethyl-1-(3-(4-methylpiperazin-1-yl)propyl)-1H-indol-5-yl)oxy)phenyl)(4-(3-fluorophenethyl)piperazin-1-yl)methanone
(4-((2,3-dimethyl-1-(3-(4-methylpiperazin-1-yl)propyl)-1H-indol-5-yl)oxy)phenyl)(4-(3-methoxyphenethyl)piperazin-1-yl)methanone
(4-((2,3-dimethyl-1-(3-(4-methylpiperazin-1-yl)propyl)-1H-indol-5-yl)oxy)phenyl)(4-phenethylpiperazin-1-yl)methanone
(4-((2,3-dimethyl-1-(3-(4-methylpiperazin-1-yl)propyl)-1H-indol-5-yl)amino)phenyl)(4-(4-fluorophenethyl)piperazin-1-yl)methanone
(4-((2,3-dimethyl-1-(3-(4-methylpiperazin-1-yl)propyl)-1H-indol-5-yl)amino)phenyl)(4-(4-methoxyphenethyl)piperazin-1-yl)methanone
(4-(2-(benzo[d][1,3]dioxol-5-yl)ethyl)piperazin-1-yl)(4-((2,3-dimethyl-1-(3-(4-methylpiperazin-1-yl)propyl)-1H-indol-5-yl)amino)phenyl)methanone
(4-((2,3-dimethyl-1-(3-(4-methylpiperazin-1-yl)propyl)-1H-indol-5-yl)amino)phenyl)(4-(3-fluorophenethyl)piperazin-1-yl)methanone
(4-((2,3-dimethyl-1-(3-(4-methylpiperazin-1-yl)propyl)-1H-indol-5-yl)amino)phenyl)(4-(3-methoxyphenethyl)piperazin-1-yl)methanone
(4-((2,3-dimethyl-1-(3-(4-methylpiperazin-1-yl)propyl)-1H-indol-5-yl)amino)phenyl)(4-phenethylpiperazin-1-yl)methanone
(3-((2,3-dimethyl-1-(3-(4-methylpiperazin-1-yl)propyl)-1H-indol-5-yl)methyl)phenyl)(4-(2-(naphthalen-2-yl)ethyl)piperazin-1-yl)methanone
3-((2,3-dimethyl-1-(3-(4-methylpiperazin-1-yl)propyl)-1H-indol-5-yl)methyl)phenyl)(4-(2-(6-methoxynaphthalen-2-yl)ethyl)piperazin-1-yl)methanone
(3-((2,3-dimethyl-1-(3-(4-methylpiperazin-1-yl)propyl)-1H-indol-5-yl)methyl)phenyl)(4-(2-(naphthalen-1-yl)ethyl)piperazin-1-yl)methanone
(3-((2,3-dimethyl-1-(3-(4-methylpiperazin-1-yl)propyl)-1H-indol-5-yl)methyl)phenyl)(4-(2-(7-methoxynaphthalen-1-yl)ethyl)piperazin-1-yl)methanone
(3-((2,3-dimethyl-1-(3-(4-methylpiperazin-1-yl)propyl)-1H-indol-5-yl)methyl)phenyl)(4-(2-(quinolin-6-yl)ethyl)piperazin-1-yl)methanone
(4-((2,3-dimethyl-1-(3-(4-methylpiperazin-1-yl)propyl)-1H-indol-5-yl)methyl)phenyl)(4-(2-(naphthalen-2-yl)ethyl)piperazin-1-yl)methanone
(4-((2,3-dimethyl-1-(3-(4-methylpiperazin-1-yl)propyl)-1H-indol-5-yl)methyl)phenyl)(4-(2-(6-methoxynaphthalen-2-yl)ethyl)piperazin-1-yl)methanone
(4-((2,3-dimethyl-1-(3-(4-methylpiperazin-1-yl)propyl)-1H-indol-5-yl)methyl)phenyl)(4-(2-(naphthalen-1-yl)ethyl)piperazin-1-yl)methanone
(4-((2,3-dimethyl-1-(3-(4-methylpiperazin-1-yl)propyl)-1H-indol-5-yl)methyl)phenyl)(4-(2-(7-methoxynaphthalen-1-yl)ethyl)piperazin-1-yl)methanone
(4-((2,3-dimethyl-1-(3-(4-methylpiperazin-1-yl)propyl)-1H-indol-5-yl)methyl)phenyl)(4-(2-(quinolin-6-yl)ethyl)piperazin-1-yl)methanone
(3-((2,3-dimethyl-1-(3-(4-methylpiperazin-1-yl)propyl)-1H-indol-5-yl)methyl)phenyl)(4-(2-(quinolin-7-yl)ethyl)piperazin-1-yl)methanone
(4-((2,3-dimethyl-1-(3-(4-methylpiperazin-1-yl)propyl)-1H-indol-5-yl)methyl)phenyl)(4-(2-(quinolin-7-yl)ethyl)piperazin-1-yl)methanone.

In a second aspect the invention relates to a pharmaceutical composition comprising a compound of formula (I) together with a pharmaceutically acceptable carrier, diluent or excipient.

Compounds and pharmaceutical compositions according to the present invention may be suitable for the treatment or prevention of a proliferative disease. Accordingly, in another aspect the invention relates to a method of treating or preventing a proliferative disease in a subject, the method comprising administering to the subject an effective amount of a compound of formula (I) according to the first aspect of the invention or a pharmaceutical composition according to the second aspect of the invention.

In a further aspect the present invention relates to the use of a compound of formula (I) according to the first aspect of the invention or a pharmaceutical composition according to the second aspect of the invention in the manufacture of a medicament for treating or preventing a proliferative disease.

In a further aspect the present invention relates to the use of a compound of formula (I) according to the first aspect of the invention or a pharmaceutical composition according to the second aspect of the invention for the treatment or prevention of a proliferative disease in a subject.

In a further aspect the present invention relates to a compound of formula (I) according to the first aspect of the invention or a pharmaceutical composition according to the second aspect of the invention for use in the treatment or prevention of a proliferative disease in a subject.

In one or more preferred embodiments, the proliferative disease is cancer, preferably a solid tumour. In various preferred embodiments, the cancer is selected from the group consisting of breast cancer, lung cancer, prostate cancer, ovarian cancer, uterine cancer brain cancer, skin cancer, colon cancer and bladder cancer.

Those skilled in the art will understand that in the context of the present invention an ‘effective amount’ is an amount sufficient to produce a desired therapeutic or pharmacological effect in the subject being treated.

In a further aspect the invention relates to a method of completely or partially preventing the recurrence of a solid tumour in a subject, the method comprising administering to the subject an effective amount of a compound of formula (I) according to the first aspect of the invention or a pharmaceutical composition according to the second aspect of the invention.

In another aspect the invention relates to the use of a compound according to the first aspect of the invention or the pharmaceutical composition according to the second aspect of the invention in the manufacture of a medicament for completely or partially preventing the recurrence of a solid tumour.

In a further aspect the present invention relates to the use of a compound of formula (I) according to the first aspect of the invention or a pharmaceutical composition according to the second aspect of the invention for completely or partially preventing the recurrence of a solid tumour in a subject.

In a further aspect the present invention relates to a compound of formula (I) according to the first aspect of the invention or a pharmaceutical composition according to the second aspect of the invention for use in completely or partially preventing the recurrence of a solid tumour in a subject.

Compounds and pharmaceutical compositions according to the present invention may be suitable for the treatment or prevention of an inflammatory disease or disorder. Accordingly, in another aspect the present invention relates to a method of treating an inflammatory disease or disorder in a subject, the method comprising administering to the subject an effective amount of a compound of formula (I) according to the first aspect of the invention or a pharmaceutical composition according to the second aspect of the invention.

In a further aspect the present invention relates to the use of a compound of formula (I) according to the first aspect of the invention or a pharmaceutical composition according to the second aspect of the invention in the manufacture of a medicament for treating an inflammatory disease or disorder.

In a further aspect the present invention relates to the use of a compound of formula (I) according to the first aspect of the invention or a pharmaceutical composition according to the second aspect of the invention for the treatment of an inflammatory disease or disorder in a subject.

In a further aspect the present invention relates to a compound of formula (I) according to the first aspect of the invention or a pharmaceutical composition according to the second aspect of the invention for use in the treatment of an inflammatory disease or disorder in a subject.

The inflammatory disease or disorder may be osteoarthritis, inflammatory bowel disease (e.g. ulcerative colitis and Crohn's disease), ulcerative proctitis, distal colitis, an autoimmune disorder (e.g. SLE, rheumatoid arthritis, glomerulonephritis), asthma or a disease involving pulmonary inflammation, or a cardiovascular disorder (e.g. atherosclerosis, hypertension and lipid dyscrasia).

The compounds of formula (I) may be used in therapy alone or in combination with one or more other agents (e.g. chemotherapeutic or anti-inflammatory agents), for example, as part of a combination therapy.

In another aspect the present invention relates to a process for preparing a compound of formula (I) comprising the steps of:

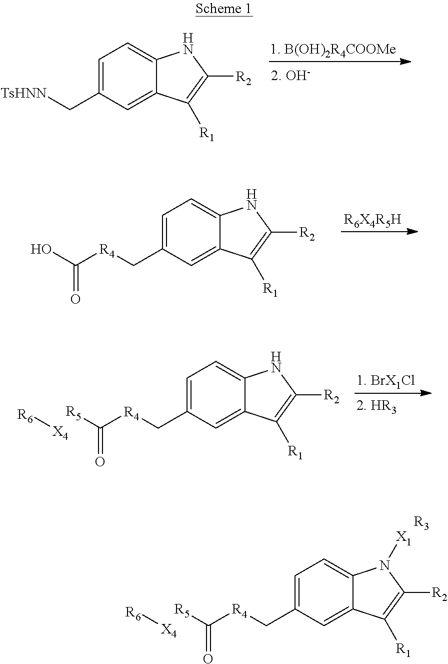

In another aspect the present invention relates to a process for preparing a compound of formula (I) comprising the steps of:

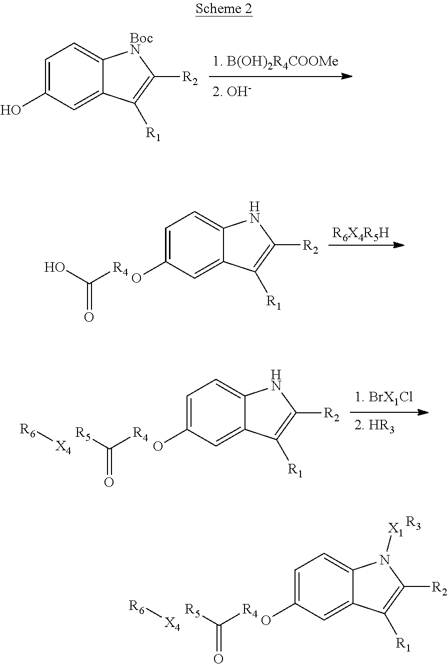

In another aspect the present invention relates to a process for preparing a compound of formula (I) comprising the steps of:

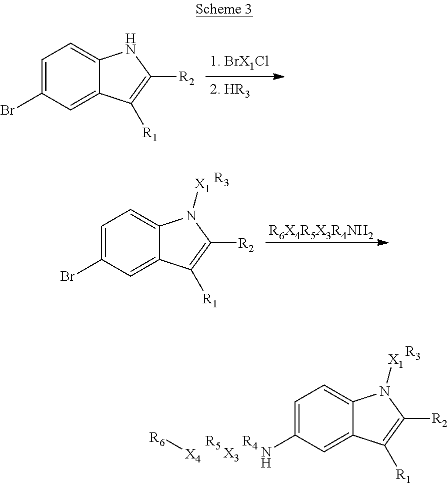

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Imaging and quantitation of actin filaments in SK-N-SH neuroblastoma cells treated with compound (A) 3504, (B) 3507 and (C) 3516. Cells were stained with 488-Atto-Phallodin and DAPI to visualize the actin filament bundles and the nucleus, respectively. Shown in the top panel is a representative grey scale immunofluorescent image from control (vehicle alone), 5 μM and 10 μM treated cells. The middle panel (enlarged inset bottom panel) shows the overlay of the cell image with the linear feature quantitation. The coloured lines indicate the detected actin filaments. Also shown is the quantitation of cell number, filament number/cell and filament number/cell area (μM²). Statistical analysis was performed using a one way ANNOVA-multiple comparison where each drug treated group was compared to the control. **** p<0.0001, **** p<0.001, *** p<0.01, ** p<0.1.

FIG. 2: Imaging and quantitation of actin filaments in SK-N-SH neuroblastoma cells treated with compound (A) 3504, (B) 3507 and (C) 3516. Cells were stained with γ9d (sheep polycolonal, 1:100) followed by 488-conjugated secondary (1:1000) and DAPI to visualize the Tpm3.1 containing filament bundles and the nucleus, respectively. Shown in the top panel is a representative grey scale immunofluorescent image from control (vehicle alone), 5 μM and 10 μM treated cells. The middle panel (enlarged inset bottom panel) shows the overlay of the cell image with the linear feature quantitation. The coloured lines indicate the detected actin filaments. Also shown is the quantitation of cell number, filament number/cell and filament number/cell area (μM²). Statistical analysis was performed using a one way ANNOVA-multiple comparison where each drug treated group was compared to the control. **** p<0.0001, **** p<0.001, *** p<0.01, ** p<0.1.

FIG. 3: Impact of compound 3507 on Tpm3.1-regulated actin-filament depolymerisation kinetics. (A and B) Depolymerisation time course of 6 μM actin filaments (35% pyrene labelled) diluted 12-fold into F-actin buffer (100 mM NaCl, 10 mM Tris-HCl pH 7.0, 2 mM MgCl₂, 1 mM EGTA, 0.2 mM CaCl₂, 0.2 mM ATP, 0.5 mM DTT, 0.01% (v/v) NaN₃) in the presence or absence of saturating amounts (10 μM) of Tpm3.1. Final concentration of F-actin and Tpm3.1 was 0.5 μM and 0.83 μM respectively. Tpm3.1 was pre-incubated with 50 μM 3507 or 1% (v/v) DMSO prior to mixing with F-actin. Depolymerisation data is normalized to the initial fluorescence value. (C and D) Initial rates (V₀) of depolymerisation for F-actin alone or Tpm3.1/F-actin, in the presence of compound 4015 or 4093. Initial rates of depolymerisation were determined from the first 3600 s, fitted to a linear regression model. Data represents mean±SEM, averaged from n>6 replicates. **** p<0.0001.

FIG. 4: Compound 3507/30% w/v Dexolve-7 was administered IP daily at 150 mg/kg for 18 days in a flank xenograft model of neuroblastoma (CHLA-20). Tumour volume was measured every 2-3 days.

FIG. 5: Compound 3507/30% w/v Dexolve-7 was administered IV 2×/week at 60 mg/kg for 14 days in a flank xenograft model of melanoma (A375). Tumour volume was measured every 2-3 days. ** p<0.01.

### DETAILED DESCRIPTION OF THE EMBODIMENTS

The invention is based on the surprising finding that compounds of general formula (I) effectively inhibit tropomyosin, which results in unexpected improvement in the treatment of proliferative diseases, particularly cancer. The development of the actin cytoskeleton involves a number of ancillary control and regulatory proteins. Identification and specific targeting of actin regulatory proteins associated with the cytoskeleton of cancer cells offers the opportunity to develop cancer specific drugs without unwanted side effects.

Actin filaments are constructed through the polymerisation of globular actin protein monomers. The actin monomer is polar, with one end bearing a positive charge and the other end a negative charge. The actin filaments thus have all the actin proteins aligned in one direction. These filaments have secondary coiled proteins, tropomyosins, associated with them. The tropomyosins play an integral role in regulating the function of actin filaments. Structurally the actin filaments are made up of polymeric actin monomers with tropomyosin dimers sitting in the alpha helical groove of the actin filament to form a homopolymer. There are more than 40 mammalian tropomyosin isoforms, each of which regulates specific actin filaments. There are specific isoforms of tropomyosins that regulate the cytoskeleton of cancer cells; disruption of this interaction offers a basis to specifically treat cancer cells.

### 1. Definitions

The following are some definitions of terms used in the art that may be helpful in understanding the description of the present invention. These are intended as general definitions and should in no way limit the scope of the present invention to those terms alone, but are put forth for a better understanding of the following description.

Unless the context requires otherwise or specifically states to the contrary, integers, steps, or elements of the invention recited herein as singular integers, steps or elements clearly encompass both singular and plural forms of the recited integers, steps or elements.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps, features, compositions and compounds.

The terms “comprising” and “including” are used herein in their open-ended and non-limiting sense unless otherwise noted.

The term “optionally substituted” as used throughout the specification denotes that the group may or may not be further substituted or fused (so as to form a polycyclic system), with one or more non-hydrogen substituent groups. Suitable chemically viable optional substituents for a particular functional group will be apparent to those skilled in the art. Typical optional substituents include C₁-C₄ alkyl, C₂-C₄ alkenyl, OH, halogen, O(C₁-C₄ alkyl), NR^{a}R^{b} wherein R^{a} and R^{b} are independently selected from H, C₁-C₃ alkyl, CONH₂, SH, S(C₁-C₃ alkyl), —CH₂—O(C₁₋₃alkyl), C₆₋₁₀ aryl, —CH₂-phenyl, hydroxyl-(C₁₋₃ alkyl), and halo-(C₁₋₃alkyl). Presently preferred optional substituents include C₁₋₃ alkyl, C₁₋₃ alkoxy, —CH₂—(C₁₋₃)alkoxy, C₆₋₁₀ aryl, —CH₂-phenyl, halogen, OH, hydroxy-(C₁₋₃)alkyl, and halo-(C₁₋₃)alkyl, e.g, CF₃, CH₂CF₃.

“Acyl” means an alkyl-CO— group in which the alkyl group is as described herein. Examples of acyl include acetyl and benzoyl. The alkyl group may be a C₁-C₆ alkyl, C₁-C₄ alkyl, or C₁-C₃ alkyl group. The group may be a terminal group or a bridging group.

“Alkyl” as a group or part of a group refers to a straight or branched aliphatic hydrocarbon group having 1-12 carbon atoms, or 1-10 carbon atoms, or 1-6 carbon atoms, or 1-4 carbon atoms, or 1-3 carbon atoms. Thus, for example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, 2-ethylpentyl, 3-ethylpentyl, heptyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, 5-methylheptyl, 1-methylheptyl, octyl, nonyl, decyl, and the like. The group may be a terminal group or a bridging group.

“Alkenyl” as a group or part of a group denotes an aliphatic hydrocarbon group containing at least one carbon-carbon double bond and which may be straight or branched such as a group having 2-12 carbon atoms, or 2-6 carbon atoms, or 2-4 carbon atoms, in the normal chain. The group may contain a plurality of double bonds in the normal chain and the orientation about each double bond is independently cis or trans, E or Z. Exemplary alkenyl groups include, but are not limited to, ethenyl, vinyl, allyl, 1-methylvinyl, 1-propenyl, 2-propenyl, 2-methyl-1-propenyl, 2-methyl-1-propenyl, 1-butenyl, 2-butenyl, 3-butentyl, 1,3-butadienyl, 1-pentenyl, 2-pententyl, 3-pentenyl, 4-pentenyl, 1,3-pentadienyl, 2,4-pentadienyl, 1,4-pentadienyl, 3-methyl-2-butenyl, 1-hexenyl, 2-hexenyl, 3-hexenyl, 1,3-hexadienyl, 1,4-hexadienyl, 2-methylpentenyl, 1-heptenyl, 2-heptentyl, 3-heptenyl, 1-octenyl, 1-nonenyl, 1-decenyl, and the like. The group may be a terminal group or a bridging group.

“Alkenyloxy” refers to an —O— alkenyl group in which alkenyl is as defined herein. Preferred alkenyloxy groups are C₂-C₁₂ alkenyloxy groups. The group may be a terminal group or a bridging group.

The terms “alkyloxy” and “alkoxy” are synonymous and refer to an —O-alkyl group in which alkyl is defined herein. Presently preferred alkoxy groups are C₁₋₆ alkoxy or C₁₋₄ alkoxy or C₁₋₃ alkoxy. Examples include, but are not limited to, methoxy, ethoxy, n-propoxy, isopropoxy, sec-butoxy, tert-butoxy, and the like. The group may be a terminal group or a bridging group.

“Alkylamino” includes both mono-alkylamino and dialkylamino, unless specified. “Mono-alkylamino” means a —NH-alkyl group, in which alkyl is as defined above. “Dialkylamino” means a —N(alkyl)₂ group, in which each alkyl may be the same or different and are each as defined herein for alkyl. The alkyl group may be a C₁-C₆ alkyl group. The group may be a terminal group or a bridging group.

“Alkynyl” as a group or part of a group means an aliphatic hydrocarbon group containing a carbon-carbon triple bond and which may be straight or branched and may have from 2-12 carbon atoms or 2-6 carbon atoms or 2-4 carbon atoms in the normal chain. Exemplary structures include, but are not limited to, ethynyl and propynyl. The group may be a terminal group or a bridging group.

“Alkynyloxy” refers to an —O-alkynyl group in which alkynyl is as defined herein. Presently preferred alkynyloxy groups are C₂-C₆ alkynyloxy groups, C₂-C₄ alkynyloxy. The group may be a terminal group or a bridging group.

“Aryl” as a group or part of a group denotes (i) an optionally substituted monocyclic, or fused polycyclic, aromatic carbocycle (ring structure having ring atoms that are all carbon) that may have from 5-18 atoms per ring. Presently preferred aryl groups have 6-14 atoms per ring, or more preferably 6-10 atoms per ring. Examples of aryl groups include phenyl, naphthyl, phenanthryl and the like; (ii) an optionally substituted partially saturated bicyclic aromatic carbocyclic moiety in which a phenyl and a C₅₋₇ cycloalkyl or C₅₋₇ cycloalkenyl group are fused together to form a cyclic structure, such as tetrahydronaphthyl, indenyl or indanyl. The group may be a terminal group or a bridging group.

“Cycloalkenyl” means a non-aromatic monocyclic or multicyclic ring system containing at least one carbon-carbon double bond and may have from 5-10 carbon atoms per ring. Exemplary monocyclic cycloalkenyl rings include cyclopentenyl, cyclohexenyl or cycloheptenyl. The cycloalkenyl group may be substituted by one or more substituent groups. The group may be a terminal group or a bridging group.

“Cycloalkyl” refers to a saturated or partially saturated, monocyclic or fused or spiro polycyclic, carbocycle that may contain from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. The group may be a terminal group or a bridging group.

The term “carbocyclic ring” as used herein refers to a carbon-based ring system. It is intended to include aryl, cycloalkenyl, cycloalkyl, and heteroaryl groups, as defined herein.

The terms “halogen” or “halo” are synonymous and refer to fluorine, chlorine, bromine or iodine.

“Heteroaryl” either alone or as part of a group refers to groups containing an aromatic ring (such as a 5- or 6-membered aromatic ring) having one or more heteroatoms as ring atoms in the aromatic ring with the remainder of the ring atoms being carbon atoms. Suitable heteroatoms include nitrogen, oxygen and sulphur. Examples of heteroaryl include thiophene, benzothiophene, benzofuran, benzimidazole, benzoxazole, benzothiazole, benzisothiazole, naphtho[2,3-b]thiophene, furan, isoindolizine, xantholene, phenoxatine, pyrrole, imidazole, pyrazole, pyridine, pyrazine, pyrimidine, pyridazine, indole, isoindole, 1H-indazole, purine, quinoline, isoquinoline, phthalazine, naphthyridine, quinoxaline, cinnoline, carbazole, phenanthridine, acridine, phenazine, thiazole, isothiazole, phenothiazine, oxazole, isooxazole, furazane, phenoxazine, 2-, 3- or 4-pyridyl, 2-, 3-, 4-, 5-, or 8-quinolyl, 1-, 3-, 4-, or 5-isoquinolinyl 1-, 2-, or 3-indolyl, and 2-, or 3-thienyl. The group may be a terminal group or a bridging group.

The term “heteroatom” or variants such as “hetero-” as used herein refers to O, N, NH and S.

Certain compounds of the disclosed embodiments may exist as single stereoisomers, racemates, and/or mixtures of enantiomers and/or diastereomers. All such single stereoisomers, racemates and mixtures thereof, are intended to be within the scope of the subject matter described and claimed.

Additionally, formula (I) is intended to cover, where applicable, solvated as well as unsolvated forms of the compounds. Thus, formula (I) includes compounds having the indicated structure, including the hydrated or solvated form, as well as the non-hydrated and non-solvated forms.

The term “pharmaceutically acceptable salt” refers to those salts which, within the scope of sound medical judgement, are suitable for use in contact with the tissues of humans and animals without undue toxicity, irritation, allergic response and the like, and are commensurate with a reasonable benefit/risk ratio. Pharmaceutically acceptable salts are well known in the art. S. M. Berge et al. describe pharmaceutically acceptable salts in detail in J. Pharmaceutical Sciences, 1977, 66:1-19. The salts can be prepared in situ during the final isolation and purification of the compounds of the invention, or separately by reacting the free base function with a suitable organic acid. Suitable pharmaceutically acceptable acid addition salts of the compounds of the present invention may be prepared from an inorganic acid or from an organic acid. Examples of such inorganic acids are hydrochloric, hydrobromic, hydroiodic, nitric, carbonic, sulphuric, and phosphoric acid. Appropriate organic acids may be selected from aliphatic, cycloaliphatic, aromatic, heterocyclic carboxylic and sulphonic classes of organic acids, examples of which are formic, acetic, propionic, succinic, glycolic, gluconic, lactic, malic, tartaric, citric, ascorbic, glucoronic, fumaric, maleic, pyruvic, alkyl sulphonic, arylsulphonic, aspartic, glutamic, benzoic, anthranilic, mesylic, salicylic, p-hydroxybenzoic, phenylacetic, mandelic, ambonic, pamoic, pantothenic, sulphanilic, cyclohexylaminosulphonic, stearic, algenic, β-hydroxybutyric, galactaric, and galacturonic acids. Suitable pharmaceutically acceptable base addition salts of the compounds of the present invention include metallic salts made from lithium, sodium, potassium, magnesium, calcium, aluminium, and zinc, and organic salts made from organic bases such as choline, diethanolamine, morpholine. Alternatively, organic salts made from N,N′-dibenzylethylenediamine, chloroprocaine, choline, diethanolamine, ethylenediamine, meglumine (N-methylglucamine), procaine, ammonium salts, quaternary salts such as tetramethylammonium salt, amino acid addition salts such as salts with glycine and arginine. In the case of compounds that are solids, it will be understood by those skilled in the art that the inventive compounds, agents and salts may exist in different crystalline or polymorphic forms, all of which are intended to be within the scope of the present invention and specified formulae.

“Prodrug” means a compound which is convertible in vivo by metabolic means (e.g. by hydrolysis, reduction or oxidation) to a compound of the present invention. For example an ester prodrug of a compound of the present invention containing a hydroxyl group may be convertible by hydrolysis in vivo to the parent molecule. Suitable esters are for example, acetates, citrates, lactates, tartrates, malonates, oxalates, salicylates, propionates, succinates, fumarates, maleates, methylene-bis-β-hydroxynaphthoates, gestisates, isethionates, di-p-toluoyltartrates, methanesulphonates, ethanesulphonates, benzenesulphonates, p-toluenesulphonates, cyclohexylsulphamates and quinates.

The terms “treating”, “treatment” and “therapy” are used herein to refer to curative therapy, prophylactic therapy and preventative therapy. Thus, in the context of the present disclosure the term “treating” encompasses curing, ameliorating or tempering the severity of cancer or its associated symptoms.

“Preventing” or “prevention” means preventing the occurrence of the cancer or tempering the severity of the cancer if it develops subsequent to the administration of the compounds or pharmaceutical compositions of the present invention. This prevents the onset of clinically evident unwanted cell proliferation altogether or the onset of a pre-clinically evident stage of unwanted rapid cell proliferation in individuals at risk. Also intended to be encompassed by this definition is the prevention of metastases of malignant cells or the arrest or reversal of the progression of malignant cells.

The terms “therapeutically effective” or “pharmacologically effective” are intended to qualify the amount of each agent which will achieve the goal of improvement in disease severity and the frequency of incidence over treatment of each agent by itself while avoiding adverse side effects typically associated with other therapies.

A “pharmaceutical carrier, diluent or excipient” includes, but is not limited to, any physiological buffered (i.e., about pH 7.0 to 7.4) medium comprising a suitable water soluble organic carrier, conventional solvents, dispersion media, fillers, solid carriers, coatings, antibacterial and antifungal agents, isotonic and absorption delaying agents. Suitable water soluble organic carriers include, but are not limited to saline, dextrose, corn oil, dimethylsulphoxide, and gelatine capsules. Other conventional additives include lactose, mannitol, corn starch, potato starch, binders such as crystalline cellulose, cellulose derivatives, acacia, gelatines, disintegrators such as sodium carboxymethyl-cellulose, and lubricants such as talc or magnesium stearate.

“Subject” includes any human or non-human animal. Thus, in addition to being useful for human treatment, the compounds of the present invention may also be useful for veterinary treatment of mammals, including companion animals and farm animals, such as, but not limited to dogs, cats, horses, cows, sheep, and pigs.

In the context of this specification the term “administering” and variations of that term including “administer” and “administration”, includes contacting, applying, delivering or providing a compound or composition of the invention to an organism, or a surface by any appropriate means.

### II. Synthesis of Compounds of the Invention

The present invention relates to functionalized indole compounds of general formula (I) as defined herein, and to the use of such compounds as therapeutic agents.

Compounds of general formula (I), or salts, hydrates or solvates, thereof may be prepared by methods known to those skilled in the art. The general synthetic scheme for preparing compounds of formula (I) is described below:

The first step in a presently preferred synthetic route for preparing compounds of formula (I) is the ligation of the indole scaffold with one of a number of linking groups.

Specific conditions being used for compounds linked with C or O groups are shown in Scheme 4.

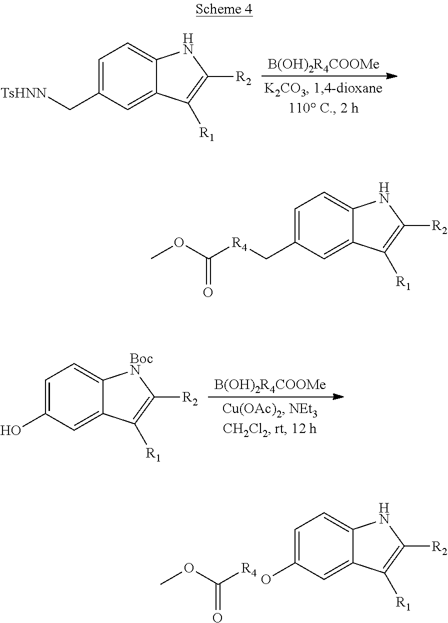

The next step is N-alkylation of the substituted indole, as show in Scheme 5. Alternatively, the N-alkylation can be performed prior to ligation of the linking group.

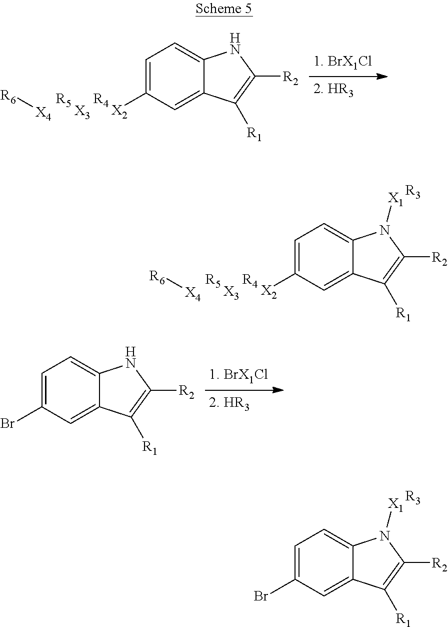

The N-alkylated indole can be further ligated with a number of linking groups, specific conditions being used for N-linked compounds as shown in Scheme 6.

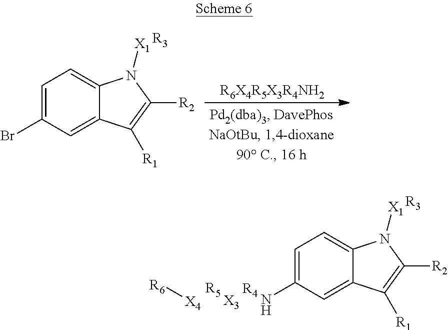

The methods described above in Schemes 4-6 may offer one or more advantages including high yields, control of stereochemistry, few synthetic steps and reaction conditions that are amenable to large scale manufacture.

The methods described above are merely representative and routine modifications and variations that would be apparent to persons skilled in the art fall within the broad scope and ambit of the invention disclosed herein.

### III. Methods of Treatment Using Compounds of the Invention

The compounds of general formula (I) according to the present invention, and pharmaceutical compositions thereof, may be used in the treatment or prevention of proliferative diseases, preferably cancer. The compounds and compositions of the invention may be useful for the treatment of a wide variety of cancers (tumours), including but not limited to, solid tumours, such as for example, breast cancer, lung cancer, prostate cancer, ovarian cancer, uterine cancer brain cancer, skin cancer, colon cancer and bladder cancer.

Advantageously, compounds of the present invention may possess superior pharmaceutical properties, such as improved resistance to conjugation via glucuronyl transferases and other water solubilizing transferases such as sulphases, which may be over-expressed on proliferative cells such as cancer cells. This may advantageously confer superior pharmaceutical properties, such as an enhanced pharmacokinetic profile through reduced conjugation and elimination.

Pharmaceutical compositions suitable for the delivery of compounds of the present invention and methods for their preparation will be readily apparent to those skilled in the art. Such compositions and methods for their preparation may be found, for example, in Reminqton's Pharmaceutical Sciences, 19th Edition (Mack Publishing Company, 1995).

The compounds or pharmaceutical compositions of the present invention may be administered orally, intravenously, intranasally, rectally, parenterally, subcutaneously, intramuscularly, topically or by any means which delivers an effective amount of the active agent to the tissue or site to be treated. It will be appreciated that different dosages may be required for treating different disorders. An effective amount of an agent is that amount which causes a statistically significant decrease in neoplastic cell count, growth, or size. Neoplastic disorders responsive to the agents of the present invention include, but are not limited to, breast cancer.

The dosage form and amount of the compounds or pharmaceutical compositions of the present invention can be readily established by reference to known treatment or prophylactic regimens.

For example, the compounds and pharmaceutical compositions may be formulated for oral, injectable, rectal, parenteral, subcutaneous, intravenous or intramuscular delivery. Non-limiting examples of particular formulation types include tablets, capsules, caplets, powders, granules, injectables, ampoules, vials, ready-to-use solutions or suspensions, lyophilized materials, suppositories and implants. The solid formulations such as the tablets or capsules may contain any number of suitable pharmaceutically acceptable excipients or carriers described above.

For intravenous, intramuscular, subcutaneous, or intraperitoneal administration, one or more compounds may be combined with a sterile aqueous solution which is preferably isotonic with the blood of the recipient. Such formulations may be prepared by dissolving solid active ingredient in water containing physiologically compatible substances such as sodium chloride or glycine, and having a buffered pH compatible with physiological conditions to produce an aqueous solution, and rendering said solution sterile. Suitable formulations may include cyclodextrins (e.g. sulfobutyl-ether-beta-cyclodextrin, or SBECD, commercially-available as Dexolve, or the formulation aid known as Captisol). The formulations may be present in unit or multi-dose containers such as sealed ampoules or vials.

The amount of therapeutically effective compound that is administered and the dosage regimen for treating a disease condition with the compounds and/or pharmaceutical compositions of the invention depends on a variety of factors, including the age, weight, sex, and medical condition of the subject, the severity of the disease, the route and frequency of administration, the particular compound employed, the location of the unwanted proliferating cells, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

The compounds of the present invention may be administered along with a pharmaceutical carrier, diluent or excipient as described above. Alternatively, or in addition to, the compounds may be administered in combination with other agents, for example, chemotherapeutic or immune-stimulating drugs or therapeutic agents.

The terms “combination therapy” or “adjunct therapy” in defining use of a compound of the present invention and one or more other pharmaceutical agents, are intended to embrace administration of each agent in a sequential manner in a regimen that will provide beneficial effects of the drug combination, and is intended as well to embrace co-administration of these agents in a substantially simultaneous manner, such as in a single formulation having a fixed ratio of these active agents, or in multiple, separate formulations of each agent.

In accordance with various embodiments of the present invention one or more compounds of general formula (I) may be formulated or administered in combination with one or more other therapeutic agents. Thus, in accordance with various embodiments of the present invention, one or more compounds of general formula (I) may be included in combination treatment regimens with surgery and/or other known treatments or therapeutic agents, such as other anticancer agents, in particular, chemotherapeutic agents, radiotherapeutic agents, and/or adjuvant or prophylactic agents.

There are large numbers of antineoplastic agents available in commercial use, in clinical evaluation and in pre-clinical development, which could be selected for treatment of cancers or other neoplasias by combination drug chemotherapy. Such anti-neoplastic agents fall into several major categories, namely, antibiotic-type agents, alkylating agents, antimetabolite agents, hormonal agents, immunological agents, interferon-type agents and a category of miscellaneous agents. Alternatively, other anti-neoplastic agents, such as metallomatrix proteases inhibitors may be used. Suitable agents which may be used in combination therapy will be recognized by those of skill in the art. Suitable agents are listed, for example, in the Merck Index, An Encyclopaedia of Chemicals, Drugs and Biologicals, 12^{th} Ed., 1996, the entire contents of which are incorporated herein by reference.

Combination regimens may involve the active agents being administered together, sequentially, or spaced apart as appropriate in each case. Combinations of active agents including compounds of the invention may be synergistic.

The co-administration of compounds of the general formula (I) may be effected by a compound of the general formula (I) being in the same unit dose as a chemotherapeutic or other anti-cancer agent, or the compound of the general formula (I) and the chemotherapeutic or other anti-cancer agents may be present in individual and discrete unit doses administered at the same, or at a similar time. Sequential administration may be in any order as required, and may require an ongoing physiological effect of the first or initial compound to be current when the second or later compound is administered, especially where a cumulative or synergistic effect is desired.

Embodiments of the invention will now be discussed in more detail with reference to the examples which is provided for exemplification only and which should not be considered limiting on the scope of the invention in any way.

### EXAMPLES

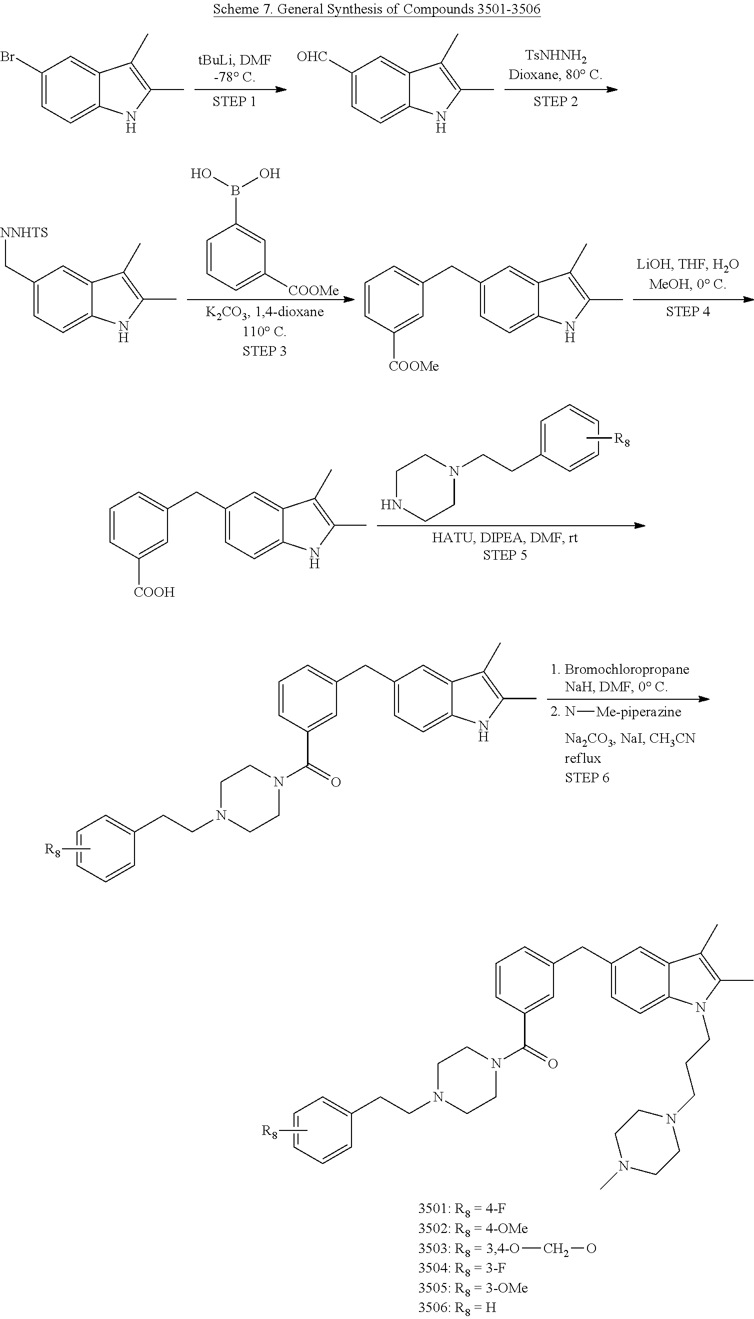

### Step 1: Preparation of 2, 3-dimethyl-1H-indole-5-carbaldehyde

To a stirred solution of 5-bromo-2,3-dimethyl-1H-indole (5.0 g, 22.42 mmol), in dry THF (50 mL) was added t-BuLi (44.8 mL, 67.20 mmol) at −78° C. The resulting reaction mixture was stirred at same temperature for 1 hour. Then dry DMF (5.0 mL, 65.00 mmol) was added to the reaction mass at −78° C. The temperature was maintained for a further 2 hours. After complete consumption of the starting material, the reaction mass was quenched with saturated ammonium chloride solution at −40° C. and extracted with ethyl acetate. The combined organic layers were washed with water and brine, dried over anhydrous Na₂SO₄ and concentrated under reduced pressure to afford the crude product. The crude compound was purified by flash column chromatography using 20-25% ethyl acetate in petroleum ether as an eluent to obtain 2,3-dimethyl-1H-indole-5-carbaldehyde as a yellow solid (3.0 g, 77%). LCMS: m/z 174.0 [M+H]⁺.

### Steps 2 and 3: Preparation of methyl 3-((2,3-dimethyl-1H-indol-5-yl)methyl)benzoate

Tosyl hydrazine (5.36 g, 28.9 mmol) was added to a stirred solution of 2,3-dimethyl-1H-indole-5-carbaldehyde (5.0 g, 28.9 mmol) in dry 1,4-dioxane (100 mL) at room temperature. The temperature was increased to 80° C. and maintained for 2 hours before cooling to 0° C.

To the crude 2,3-dimethyl-5-((1-tosyl-2λ²-diazanyl)methyl)-1H-indole in the reaction mass was added K₂CO₃ (5.96 g, 43.2 mmol) and (3-(methoxycarbonyl)phenyl)boronic acid (5.18 g, 28.8 mmol). The reaction temperature was raised to 110° C. and maintained for 4 hours. After complete consumption of the starting material, the reaction mass was concentrated, diluted with water and extracted with ethyl acetate. The combined organic layers were washed with water and brine, dried over anhydrous Na₂SO₄ and concentrated under reduced pressure to afford the crude product. The crude compound was purified by flash column chromatography using 20-25% ethyl acetate in petroleum ether as an eluent to obtain methyl 3-((2,3-dimethyl-1H-indol-5-yl)methyl)benzoate as a brown solid (4.0 g, 47%). LCMS: m/z 294.38 [M+H]⁺.

### Step 4: Preparation of 3-((2,3-dimethyl-1H-indol-5-yl)methyl)benzoic acid

Methyl 3-((2,3-dimethyl-1H-indol-5-yl)methyl)benzoate (4.0 g, 13.60 mmol) was dissolved in a THF: H₂O: MeOH (6:2:2) mixture. LiOH.H₂O (1.14 g, 27.20 mmol) was added at 0° C. The reaction mixture was allowed to stir at room temperature for 16 hours. After complete consumption of the starting material, the reaction mass was concentrated and then partitioned between ethyl acetate and water. The aqueous layer was collected and acidified with saturated citric acid solution at 0° C. The obtained solid was filtered and dried over vacuum to afford 3-((2,3-dimethyl-1H-indol-5-yl)methyl)benzoic acid as a brown solid (2.9 g, 77%). LCMS: m/z 280.39 [M+H]⁺.

### Step 5: Preparation of (3-((2,3-dimethyl-1H-indol-5-yl)methyl)phenyl) (4-(4-fluorophenethyl)piperazin-1-yl)methanone

To a stirred solution of 3-((2,3-dimethyl-1H-indol-5-yl)methyl)benzoic acid (300 mg, 1.08 mmol) in DMF (5 mL), DIPEA (0.5 mL) was added then stirred for 10 minutes, followed by the addition of HATU (817.6 mg, 2.150 mmol) and stirring for 30 minutes. The reaction mass was cooled to 0° C. and 1-(4-fluorophenethyl)piperazine (246.2 mg, 1.183 mmol) was added. The mixture was then stirred at room temperature overnight. After complete consumption of the starting material, the reaction mixture was poured into ice water. The resulting precipitate was collected by filtration and dried to afford (3-((2,3-dimethyl-1H-indol-5-yl)methyl)phenyl)(4-(4-fluorophenethyl)piperazin-1-yl)methanone (300 mg, 60%). LCMS: m/z 470.23 [M+H]⁺.

Other analogues prepared via this method:
3-((2,3-dimethyl-1H-indol-5-yl)methyl)phenyl)(4-(4-methoxyphenethyl)piperazin-1-yl)methanone (58%). LCMS: m/z 482.47[M+H]⁺.
(4-(2-(benzo[d][1,3]dioxol-5-yl)ethyl)piperazin-1-yl)(3-((2,3-dimethyl-1H-indol-5-yl)methyl)phenyl)methanone (56%). LCMS: m/z 496.48 [M+H]⁺.
(3-((2,3-dimethyl-1H-indol-5-yl)methyl)phenyl)(4-(3-fluorophenethyl)piperazin-1-yl)methanone (70%). LCMS: m/z 470.32 [M+H]⁺.
3-((2,3-dimethyl-1H-indol-5-yl)methyl)phenyl)(4-(3-methoxyphenethyl)piperazin-1-yl)methanone (72%). LCMS: m/z 482.41 [M+H]⁺.
3-((2,3-dimethyl-1H-indol-5-yl)methyl)phenyl)(4-phenethylpiperazin-1-yl)methanone (62%). LCMS: m/z 452.23 [M+H]⁺.

### Step 6-1: Preparation of (3-((2,3-dimethyl-1-(3-chloropropyl)-1H-indol-5-yl)methyl)phenyl) (4-(4-fluorophenethyl)piperazin-1-yl) methanone

NaH (30.6 mg, 1.2779 mmol) was added portionwise to a stirred solution of (3-((2,3-dimethyl-1H-indol-5-yl)methyl)phenyl)(4-(4-fluorophenethyl)piperazin-1-yl)methanone (300 mg, 0.6389 mmol) in DMF (4 mL) at 0° C. The reaction mixture was allowed to warm to room temperature for 30 minutes. Bromochloropropane (0.13 mL, 1.2779 mmol) was added dropwise at 0° C. and the mixture was allowed to stir at room temperature for 3 hours. After complete consumption of the starting material, ice cold water was added into the reaction mixture, which was then extracted with ethyl acetate. The organic layer was washed with brine solution, dried over anhydrous Na₂SO₄ and concentrated under reduced pressure to afford the crude product. The crude compound was purified by flash column chromatography using ethyl acetate as an eluent to afford (3-((2,3-dimethyl-1-(3-chloropropyl)-1H-indol-5-yl)methyl)phenyl)(4-(4-fluorophenethyl)piperazin-1-yl)methanone as a brown gummy liquid (200 mg, 57%).

LCMS: m/z 546.0 [M+H]⁺.

Other analogues prepared via this method:
(3-((2,3-dimethyl-1-(3-chloropropyl)-1H-indol-5-yl)methyl)phenyl)(4-(4-methoxyphenethyl)piperazin-1-yl)methanone (50%). LCMS: m/z 558.0 [M+H]⁺.
(4-(2-(benzo[d][1,3]dioxol-5-yl)ethyl)piperazin-1-yl)(3-((1-(3-chloropropyl)-2,3-dimethyl-1H-indol-5-yl)methyl)phenyl)methanone (58%). LCMS: m/z 572.0 [M+H]⁺.
(3-((2,3-dimethyl-1-(3-chloropropyl)-1H-indol-5-yl)methyl)phenyl)(4-(3-fluorophenethyl)piperazin-1-yl)methanone (43%). LCMS: m/z 546.39 [M+H]⁺.
(3-((2,3-dimethyl-1-(3-chloropropyl)-1H-indol-5-yl)methyl)phenyl)(4-(3-methoxyphenethyl)piperazin-1-yl)methanone (43%). LCMS: m/z 558.45 [M+H]⁺.
(3-((2,3-dimethyl-1-(3-chloropropyl)-1H-indol-5-yl)methyl)phenyl)(4-phenethylpiperazin-1-yl)methanone (45%). LCMS: m/z 528.31 [M+H]⁺.

### Step 6-2: Preparation of Compound 3501, (3-((2,3-dimethyl-1-(3-(4-methylpiperazin-1-yl)propyl)-1H-indol-5-yl)methyl)phenyl) (4-(4-fluorophenethyl)piperazin-1-yl)methanone

To a stirred solution of (3-((2,3-dimethyl-1-(3-chloropropyl)-1H-indol-5-yl)methyl)phenyl)(4-(4-fluorophenethyl)piperazin-1-yl)methanone (200 mg, 0.366 mmol) in acetonitrile (5 mL) at room temperature, sodium iodide (137.1 mg, 0.9155 mmol) and sodium carbonate (116.4 mg, 1.0986 mmol) were added, followed by N-methylpiperazine (91.70 mg, 0.9155 mmol). The reaction mixture was heated to 75° C. for 16 hours. After complete consumption of the starting material, the reaction mixture was cooled to room temperature, diluted with ethyl acetate (60 mL), washed with water and brine solution, dried over anhydrous Na₂SO₄ and concentrated under reduced pressure to afford the crude product. The crude compound was purified by flash column chromatography using 5% methanol-CH₂Cl₂ as an eluent to afford (3-((2,3-dimethyl-1-(3-(4-methylpiperazin-1-yl)propyl)-1H-indol-5-yl)methyl)phenyl)(4-(4-fluorophenethyl)piperazin-1-yl)methanone (Compound 3501) as a pale yellow gummy solid (43 mg, 19%).

¹H NMR (300 MHz, d₆-DMSO): δ 7.37-7.30 (m, 2H), 7.28-7.22 (m, 4H), 7.19-7.13 (m, 2H), 7.09 (t, J=9.0 Hz, 2H), 6.91 (br d, J=8.3 Hz, 1H), 4.09-4.02 (m, 4H), 3.66-3.46 (m, 4H), 2.71 (t, J=7.1 Hz, 2H), 2.64-2.15 (m, 22H), 2.14 (s, 3H), 1.81-1.71 (m, 2H).

LCMS: m/z 610.56 [M+H]⁺.

Other analogues prepared via this method:

Compound 3502, (3-((2,3-dimethyl-1-(3-(4-methylpiperazin-1-yl)propyl)-1H-indol-5-yl)methyl)phenyl)(4-(4-methoxyphenethyl)piperazin-1-yl)methanone (34%).

¹H NMR (400 MHz, CD₃OD): δ 7.41-7.37 (m, 2H), 7.25 (br s, 1H), 7.23-7.19 (m, 2H), 7.15-7.07 (m, 3H), 6.92 (br d, J=6.8 Hz, 1H), 6.84 (d, J=8.8 Hz, 2H), 4.11 (t, J=6.8 Hz, 2H), 4.08 (s, 2H), 3.76 (s, 3H), 3.72 (br s, 2H), 3.46 (br s, 2H), 2.79-2.20 (m, 24H), 2.18 (s, 3H), 1.88 (quintet, J=6.8 Hz, 2H). LCMS: m/z 622.58 [M+H]⁺.

Compound 3503, (4-(2-(benzo[d][1,3]dioxol-5-yl)ethyl)piperazin-1-yl)(3-((2,3-dimethyl-1-(3-(4-methylpiperazin-1-yl)propyl)-1H-indol-5-yl)methyl)phenyl)methanone (18%).

¹H NMR (400 MHz, CD₃OD): δ 7.42-7.33 (m, 2H), 7.27-7.22 (m, 2H), 7.20 (br d, J=6.8 Hz, 1H), 7.14 (br s, 1H), 6.92 (dd, J=8.0 Hz, 1.2 Hz, 1H), 6.83-6.77 (m, 2H), 6.65 (dd, J=8.4 Hz, 1.2 Hz, 1H), 5.90 (s, 2H), 4.13 (t, J=6.8 Hz, 2H), 4.08 (s, 2H), 3.71 (br s, 2H), 3.34 (br s, 2H), 2.77 (br s, 4H), 2.70-2.65 (m, 2H), 2.59-2.47 (m, 11H), 2.36 (t, J=6.8 Hz, 2H), 2.31 (s, 3H), 2.25 (br s, 2H), 2.18 (s, 3H), 1.89 (quintet, J=6.9 Hz, 2H).

LCMS: m/z 636.54 [M+H]⁺.

Compound 3504, (3-((2,3-dimethyl-1-(3-(4-methylpiperazin-1-yl)propyl)-1H-indol-5-yl)methyl)phenyl)(4-(3-fluorophenethyl)piperazin-1-yl)methanone (12%).

¹H NMR (300 MHz, CD₃OD): δ 7.40-7.34 (m, 2H), 7.33-7.18 (m, 4H), 7.15 (br s, 1H), 7.06-6.96 (m, 2H), 6.96-6.87 (m, 2H), 4.12-4.08 (m, 4H), 3.71 (br s, 2H), 3.38 (br s, 2H), 2.81-2.71 (m, 2H), 2.71-2.47 (m, 10H), 2.46-2.21 (m, 12H), 2.32 (s, 3H), 1.87 (quintet, J=6.9 Hz, 2H). LCMS: m/z 610.6 [M+H]⁺.

Compound 3505, (3-((2,3-dimethyl-1-(3-(4-methylpiperazin-1-yl)propyl)-1H-indol-5-yl)methyl)phenyl)(4-(3-methoxyphenethyl)piperazin-1-yl)methanone (12%).

¹H NMR (300 MHz, d₆-DMSO): δ 7.35-7.32 (m, 2H), 7.28-7.25 (m, 2H), 7.21-7.13 (m, 3H), 6.91 (br d, J=8.4 Hz, 1H), 6.82-6.72 (m, 3H), 4.09-4.03 (m, 4H), 3.73 (s, 3H), 3.53 (br s, 2H), 3.25 (br s, 2H), 2.74-2.61 (m, 4H), 2.47-2.15 (m, 20H), 2.14 (s, 3H), 1.82-1.71 (m, 2H). LCMS: m/z 622.58 [M+H]⁺.

Compound 3506, (3-((2,3-dimethyl-1-(3-(4-methylpiperazin-1-yl)propyl)-1H-indol-5-yl)methyl)phenyl)(4-phenethylpiperazin-1-yl)methanone (12%).

¹H NMR (300 MHz, d₆-DMSO): δ 7.42-7.08 (m, 11H), 6.94-6.89 (m, 1H), 4.11-4.01 (m, 4H), 3.62-3.43 (m, 4H), 2.76-2.67 (m, 2H), 2.63-2.10 (m, 25H), 1.91-1.81 (m, 2H).

LCMS: m/z 592.58 [M+H]⁺.

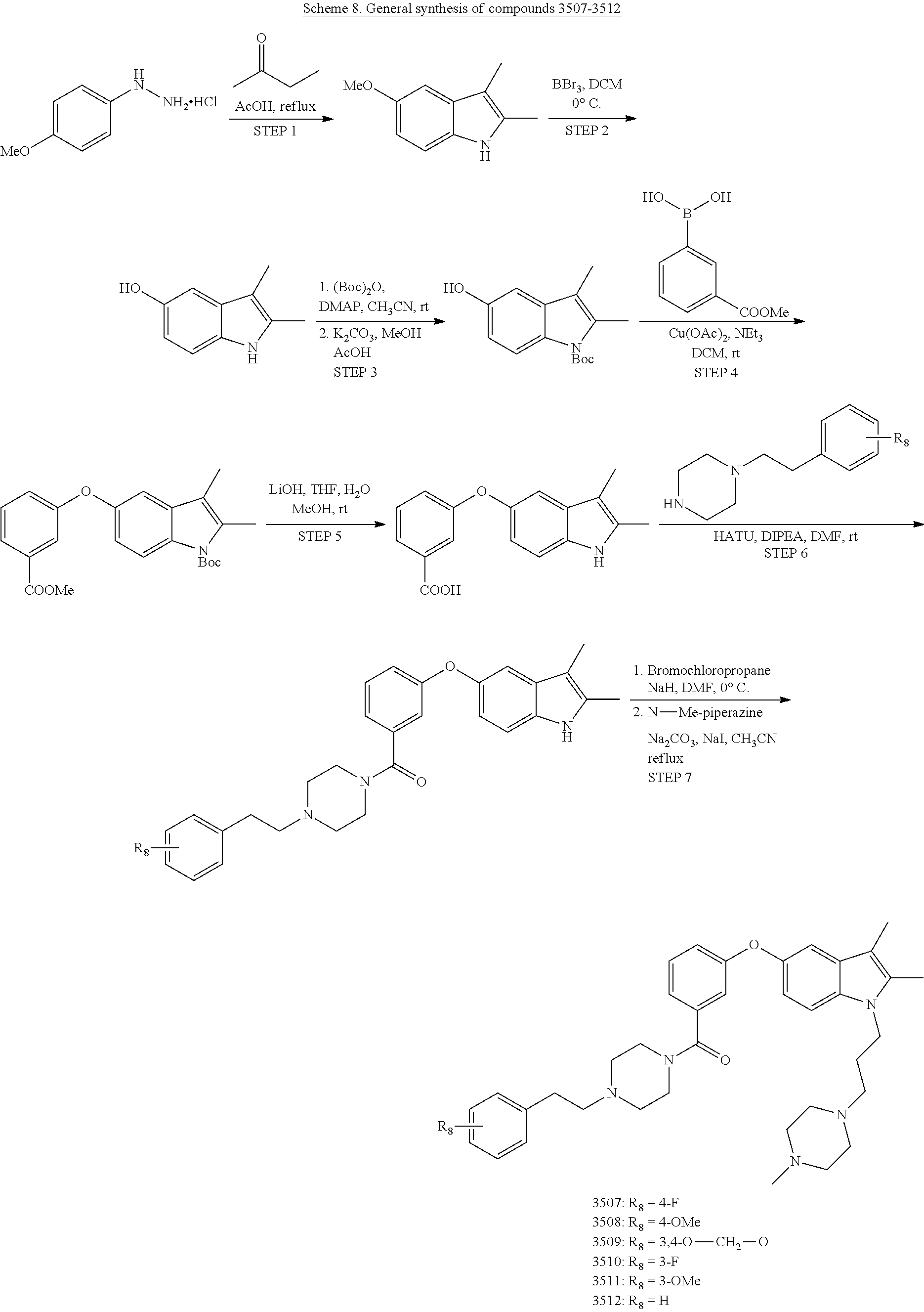

### Step 1: Preparation of 5-methoxy-2,3-dimethyl-1H-indole

2-Butanone (11.93 mL, 128.8 mmol) was added to a stirred solution of 4-methoxy hydrazine hydrochloride (15.00 g, 85.89 mmol) in acetic acid (150 mL) then heated at 80° C. for 1.5 hours. After complete consumption of the starting material, the acetic acid was removed via rotary evaporator and the reaction mass was basified using saturated NaHCO₃ solution. The resulting grey precipitate was collected by filtration and dried for 1 hour. The crude compound was purified by flash column chromatography using 20% ethyl acetate in petroleum ether as an eluent to afford 5-methoxy-2,3-dimethyl-1H-indole as a grey solid (8.8 g, 59%). LCMS: m/z 176.23 [M+H]⁺.

### Step 2: Preparation of 2,3-dimethyl-1H-indol-5-ol

BBr₃ (12.18 mL, 128.40 mmol) was added to a stirred solution of 5-methoxy-2,3-dimethyl-1H-indole in DCM (50 mL) at 0° C. The temperature was maintained at 0-5° C. for 2 hours. After complete consumption of the starting material, the reaction mixture was basified using with saturated NaHCO₃ then extracted with CH₂Cl₂. The organic layer was washed with brine solution, dried over anhydrous Na₂SO₄ and concentrated under reduced pressure to afford the crude product. The crude compound was purified by flash column chromatography using 20-50% ethyl acetate in petroleum ether as an eluent to afford 2,3-dimethyl-1H-indol-5-ol as an off white solid (8.2 g, 100%). LCMS: m/z 162.08 [M+H]⁺.

### Step 3: Preparation of tert-butyl 5-hydroxy-2,3-dimethyl-1H-indole-1-carboxylate

To a stirred solution of 2,3-dimethyl-1H-indol-5-ol (7.20 g, 44.7 mmol), in acetonitrile (72 mL) was added Boc-anhydride (29.2 g, 134 mmol) and DMAP (0.55 g, 4.472 mmol) at room temperature. The reaction mass was stirred at room temperature overnight. After complete consumption of the starting material, acetonitrile was evaporated under reduced pressure to yield a crude mixture of both the N-Boc-5-hydroxyindole and the N,O-di-Boc-protected compound (8.2 g, 51.42 mmol). The mixture was re-dissolved in methanol (828 mL), K₂CO₃ (21.3 g, 154.2 mmol) was added and the resulting mixture was stirred at room temperature for 2 hours. After completion of the reaction, the mixture was cooled to 0° C., acetic acid was added (10 mL) and the mixture was stirred for 10 minutes. The reaction mass was extracted with ethyl acetate. The organic layer was washed with water and brine solution, dried over anhydrous Na₂SO₄ and concentrated under reduced pressure to afford the crude product. The crude compound was purified on 100-200 mesh silica gel eluting with 20% ethyl acetate in petroleum ether to afford tert-butyl 5-hydroxy-2,3-dimethyl-1H-indole-1-carboxylate as a brown liquid (9.5 g, 72%). LCMS: m/z 262.40 [M+H]⁺.

### Step 4: Preparation of tert-butyl 5-(3-(methoxycarbonyl)phenoxy)-2,3-dimethyl-1H-indole-1-carboxylate

To a stirred solution of tert-butyl 5-hydroxy-2,3-dimethyl-1H-indole-1-carboxylate (7.00 g, 26.7 mmol) in DCM (100 mL) was added (3-(methoxycarbonyl)phenyl)boronic acid (14.4 g, 80.361 mmol). Cu(OAc)₂ (12.16 g, 66.96 mmol) was then added, followed by NEt₃ (18.5 ml, 133.93 mmol) and the system was purged with oxygen gas for 4 hours. The whole reaction mass was stirred under an oxygen atmosphere overnight. After complete consumption of the starting material, the reaction mass was filtered through a bed of Celite. The filtrate was diluted with water and extracted with DCM. The organic layer was washed with brine, dried over anhydrous Na₂SO₄ and concentrated under reduced pressure to afford the crude product. The crude compound was purified on 100-200 mesh silica gel eluting with 10% ethyl acetate in petroleum ether to afford tert-butyl 5-(3-(methoxycarbonyl)phenoxy)-2,3-dimethyl-1H-indole-1-carboxylate as a brown liquid (8.2 g, 77%). LCMS: m/z 396.43 [M+H]⁺.

### Step 5: Preparation of 3-((2,3-dimethyl-1H-indol-5-yl)oxy)benzoic acid

To a stirred solution of tert-butyl 5-(3-(methoxycarbonyl)phenoxy)-3-methyl-1H-indole-1-carboxylate (8.20 g, 20.8 mmol) in THF (100 mL) and water (100 mL), was added LiOH.H₂O (17.4 g, 415 mmol). The mixture was stirred at room temperature for 4 hours. After complete consumption of the starting material, THF was evaporated under reduced pressure and the reaction mass was cooled to 0° C., acidified (to pH 1) with 1 N HCl, and then extracted with ethyl acetate. The organic layers were dried over anhydrous Na₂SO₄ and concentrated under reduced pressure to afford the crude product. Trituration with n-pentane afforded pure 3-((2,3-dimethyl-1H-indol-5-yl)oxy)benzoic acid as an off white solid (5.0 g, 86%). LCMS: m/z 282.2 [M+H]⁺.

### Step 6: Preparation of (3-((2,3-dimethyl-1H-indol-5-yl)oxy)phenyl) (4-(4-fluorophenethyl)piperazin-1-yl)methanone

To a stirred solution of 3-((2,3-dimethyl-1H-indol-5-yl)oxy)benzoic acid (0.25 g, 0.88 mmol) in DMF (3 mL), DIPEA (0.70 mL, 4.44 mmol) was added. After 10 minutes stirring, HATU (0.50 g, 1.33 mmol) was added and the mixture was stirred for a further 30 minutes at room temperature. The reaction mass was cooled to 0° C., 1-(4-fluorophenethyl)piperazine (0.32 g, 1.33 mmol) was added and the reaction mixture was stirred at room temperature overnight. After complete consumption of the starting material, the reaction mixture was poured into ice water and extracted with ethyl acetate. The organic layer was washed with water and brine, dried over anhydrous Na₂SO₄ and concentrated under reduced pressure to afford the crude product. The crude compound was purified on 100-200 mesh silica eluting with 30% ethyl acetate in petroleum ether to obtain (3-((2,3-dimethyl-1H-indol-5-yl)oxy)phenyl)(4-(4-fluorophenethyl)piperazin-1-yl)methanone as a yellow solid (410 mg, 97%). LCMS: m/z 472.52 [M+H]⁺.

Other analogues prepared via this method:
(3-((2,3-dimethyl-1H-indol-5-yl)oxy)phenyl)(4-(4-methoxyphenethyl)piperazin-1-yl)methanone (92%). LCMS: m/z 484.56 [M+H]⁺.
(4-(2-(benzo[d][1,3]dioxol-5-yl)ethyl)piperazin-1-yl)(3-((2,3-dimethyl-1H-indol-5-yl)oxy)phenyl)methanone (97%). LCMS: m/z 498.50 [M+H]⁺.
(3-((2,3-dimethyl-1H-indol-5-yl)oxy)phenyl)(4-(3-fluorophenethyl)piperazin-1-yl)methanone (71%). LCMS: m/z 472.55 [M+H]⁺.
(3-((2,3-dimethyl-1H-indol-5-yl)oxy)phenyl)(4-(3-methoxyphenethyl)piperazin-1-yl)methanone (92%). LCMS: m/z 484.56 [M+H]⁺.
(3-((2,3-dimethyl-1H-indol-5-yl)oxy)phenyl)(4-phenethylpiperazin-1-yl)methanone (89%). LCMS: m/z 454.47 [M+H]⁺.

### Step 7-1: Preparation of (3-((1-(3-chloropropyl)-2,3-dimethyl-1H-indol-5-yl)oxy)phenyl) (4-(4-fluorophenethyl)piperazin-1-yl) methanone

KOtBu (0.29 g, 4.434 mmol) was added portionwise to a stirred solution of (3-((2,3-dimethyl-1H-indol-5-yl)oxy)phenyl)(4-(4-fluorophenethyl)piperazin-1-yl)methanone (0.41 g, 0.88 mmol) in DMF (5 mL) at 0° C. The mixture was allowed to warm to room temperature for 30 minutes. To this, bromochloropropane (0.43 mL, 4.43 mmol) was added dropwise at 0° C. The mixture was allowed to warm to room temperature and was stirred for 3 hours. After complete consumption of the starting material, ice-cold water was added and the reaction mixture was extracted with ethyl acetate. The organic layer was washed with brine solution, dried over anhydrous Na₂SO₄ and concentrated under reduced pressure to afford the crude product. The crude product was purified by flash column chromatography using 5% ethyl acetate in petroleum ether as an eluent to afford (3-((1-(3-chloropropyl)-2,3-dimethyl-1H-indol-5-yl)oxy)phenyl)(4-(4-fluorophenethyl)piperazin-1-yl)methanone as a brown gummy solid (130 mg, 27%).

LCMS: m/z 548.59 [M+H]⁺.

Other analogues prepared via this method:
(3-((1-(3-chloropropyl)-2,3-dimethyl-1H-indol-5-yl)oxy)phenyl)(4-(4-methoxyphenethyl)piperazin-1-yl)methanone (64%). LCMS: m/z 560.53 [M+H]⁺.
(4-(2-(benzo[d][1,3]dioxol-5-yl)ethyl)piperazin-1-yl)(3-((1-(3-chloropropyl)-2,3-dimethyl-1H-indol-5-yl)oxy)phenyl)methanone (74%). LCMS: m/z 574.89 [M+H]⁺.
(3-((1-(3-chloropropyl)-2,3-dimethyl-1H-indol-5-yl)oxy)phenyl)(4-(3-fluorophenethyl)piperazin-1-yl)methanone (39%). LCMS: m/z 548.55 [M+H]⁺.
(3-((1-(3-chloropropyl)-2,3-dimethyl-1H-indol-5-yl)oxy)phenyl)(4-(3-methoxyphenethyl)piperazin-1-yl)methanone (57%) LCMS: m/z 560.53 [M+H]⁺.
(3-((1-(3-chloropropyl)-2,3-dimethyl-1H-indol-5-yl)oxy)phenyl)(4-phenethylpiperazin-1-yl)methanone (43%) LCMS: m/z 530.41 [M+H]⁺.

### Step 7-2: Preparation of Compound 3507, (3-((2,3-dimethyl-1-(3-(4-methylpiperazin-1-yl)propyl)-1H-indol-5-yl)oxy)phenyl) (4-(4-fluorophenethyl)piperazin-1-yl)methanone

To a stirred solution of (3-((1-(3-chloropropyl)-2,3-dimethyl-1H-indol-5-yl)oxy)phenyl)(4-(4-fluorophenethyl)piperazin-1-yl)methanone (250 mg, 0.455 mmol) in acetonitrile (10 mL), were added sodium iodide (170 mg, 1.137 mmol) and sodium carbonate (241 mg, 2.27 mmol) followed by N-methylpiperazine (182 mg, 1.82 mmol) at room temperature.

The reaction mixture was heated to 75° C. for 16 hours. After complete consumption of the starting material, the reaction mixture was cooled to room temperature, diluted with ethyl acetate (30 mL), washed with water and brine, dried over anhydrous Na₂SO₄ and concentrated under reduced pressure to afford the crude product. The crude compound was purified by flash column chromatography using 5% methanol-CH₂Cl₂ as an eluent to afford (3-((2,3-dimethyl-1-(3-(4-methylpiperazin-1-yl)propyl)-1H-indol-5-yl)oxy)phenyl)(4-(4-fluorophenethyl)piperazin-1-yl)methanone (Compound 3507) as an off white solid (87 mg, 31%).

¹H NMR (300 MHz, d₆-DMSO): δ 7.42 (d, J=9.0 Hz, 1H), 7.38 (t, J=7.8 Hz, 1H), 7.24 (dd, J=8.4 Hz, 6.0 Hz, 2H), 7.12-7.06 (m, 3H), 7.02-6.96 (m, 2H), 6.81 (dd, J=8.7 Hz, 2.1 Hz, 1H), 6.76 (br s, 1H), 4.12 (t, J=6.9 Hz, 2H), 3.57 (br s, 2H), 3.33 (br s, 2H), 2.72-2.14 (m, 26H), 2.13 (s, 3H), 1.85-1.74 (m, 2H). LCMS: m/z 621.54 [M+H]⁺.

Other analogues prepared by this method:

Compound 3508, (3-((2,3-dimethyl-1-(3-(4-methylpiperazin-1-yl)propyl)-1H-indol-5-yl)oxy)phenyl)(4-(4-methoxyphenethyl)piperazin-1-yl)methanone (6%).

¹H NMR (300 MHz, d₆-DMSO): δ 7.43 (d, J=8.7 Hz, 1H), 7.38 (t, J=7.5 Hz, 1H), 7.14-7.08 (m, 3H), 7.04-6.96 (m, 2H), 6.86-6.79 (m, 3H), 6.77 (br s, 1H), 4.12 (t, J=6.9 Hz, 2H), 3.71 (s, 3H), 3.51 (br s, 4H), 2.70-2.15 (m, 24H), 2.13 (s, 3H), 1.88-1.74 (m, 2H).

LCMS: m/z 624.65 [M+H]⁺.

Compound 3509, (4-(2-(benzo[d][1,3]dioxol-5-yl)ethyl)piperazin-1-yl)(3-((2,3-dimethyl-1-(3-(4-methylpiperazin-1-yl)propyl)-1H-indol-5-yl)oxy)phenyl)methanone (35%).

¹H NMR (300 MHz, CD₃OD): δ 7.41-7.33 (m, 2H), 7.15-7.00 (m, 3H), 6.85-6.79 (m, 2H), 6.73-6.60 (m, 2H), 6.65 (br d, J=8.1 Hz, 1H), 5.89 (s, 2H), 4.16 (t, J=6.9 Hz, 2H), 3.81-3.51 (m, 2H), 3.53-3.37 (m, 2H), 2.72-2.64 (m, 2H), 2.61-2.42 (m, 10H), 2.42-2.25 (m, 12H), 2.17 (s, 3H), 1.94 (quintet, J=6.9 Hz, 2H). LCMS: m/z 638.48 [M+H]⁺.

Compound 3510, (3-((2,3-dimethyl-1-(3-(4-methylpiperazin-1-yl)propyl)-1H-indol-5-yl)oxy)phenyl)(4-(3-fluorophenethyl)piperazin-1-yl)methanone (41%).

¹H NMR (300 MHz, CD₃OD): δ 7.42-7.32 (m, 2H), 7.28 (td, J=7.8 Hz, 6.0 Hz, 1H), 7.09 (d, J=2.4 Hz, 1H), 7.07-6.87 (m, 5H), 6.84-6.78 (m, 2H), 4.17 (t, J=6.9 Hz, 2H), 3.70 (br s, 2H), 3.43 (br s, 2H), 2.83-2.77 (m, 2H), 2.64-2.32 (m, 19H), 2.31 (s, 3H), 2.17 (s, 3H), 1.91 (quintet, J=6.9 Hz, 2H). LCMS: m/z 612.51 [M+H]⁺.

Compound 3511, (3-((2,3-dimethyl-1-(3-(4-methylpiperazin-1-yl)propyl)-1H-indol-5-yl)oxy)phenyl)(4-(3-methoxyphenethyl)piperazin-1-yl)methanone (15%).

¹H NMR (300 MHz, d₆-DMSO): δ 77.38 (t, J=7.5 Hz, 1H), 7.39 (d, J=8.7 Hz, 1H), 7.18 (dd, J=8.4 Hz, 6.6 Hz, 1H), 7.09 (d, J=2.1 Hz, 1H), 7.08-7.00 (m, 2H), 6.83-6.72 (m, 5H), 4.16 (t, J=6.9 Hz, 2H), 3.77 (s, 3H), 3.71 (br s, 2H), 3.43 (br s, 2H), 2.73 (dd, J=9.6 Hz, 6.6 Hz, 2H), 2.63-2.29 (m, 19H), 2.28 (s, 3H), 2.17 (s, 3H), 1.90 (quintet, J=7.2 Hz, 2H). LCMS: m/z 624.49 [M+H]⁺.

Compound 3512, (3-((2,3-dimethyl-1-(3-(4-methylpiperazin-1-yl)propyl)-1H-indol-5-yl)oxy)phenyl)(4-phenethylpiperazin-1-yl)methanone (60%).

¹H NMR (300 MHz, d₆-DMSO): δ 7.43 (d, J=8.7 HZ, 1H), 7.38 (t, J=7.5 Hz, 1H), 7.32-7.16 (m, 5H), 7.13 (d, J=2.1 Hz, 1H), 7.03-6.96 (m, 2H), 6.82 (dd, J=8.4 Hz, 2.4 Hz, 1H), 6.77 (br s, 1H), 4.12 (t, J=6.9 Hz, 2H), 3.54 (br s, 4H), 3.03-2.18 (m, 24H), 2.14 (s, 3H), 1.88-1.64 (m, 2H). LCMS: m/z 594.52 [M+H]⁺.

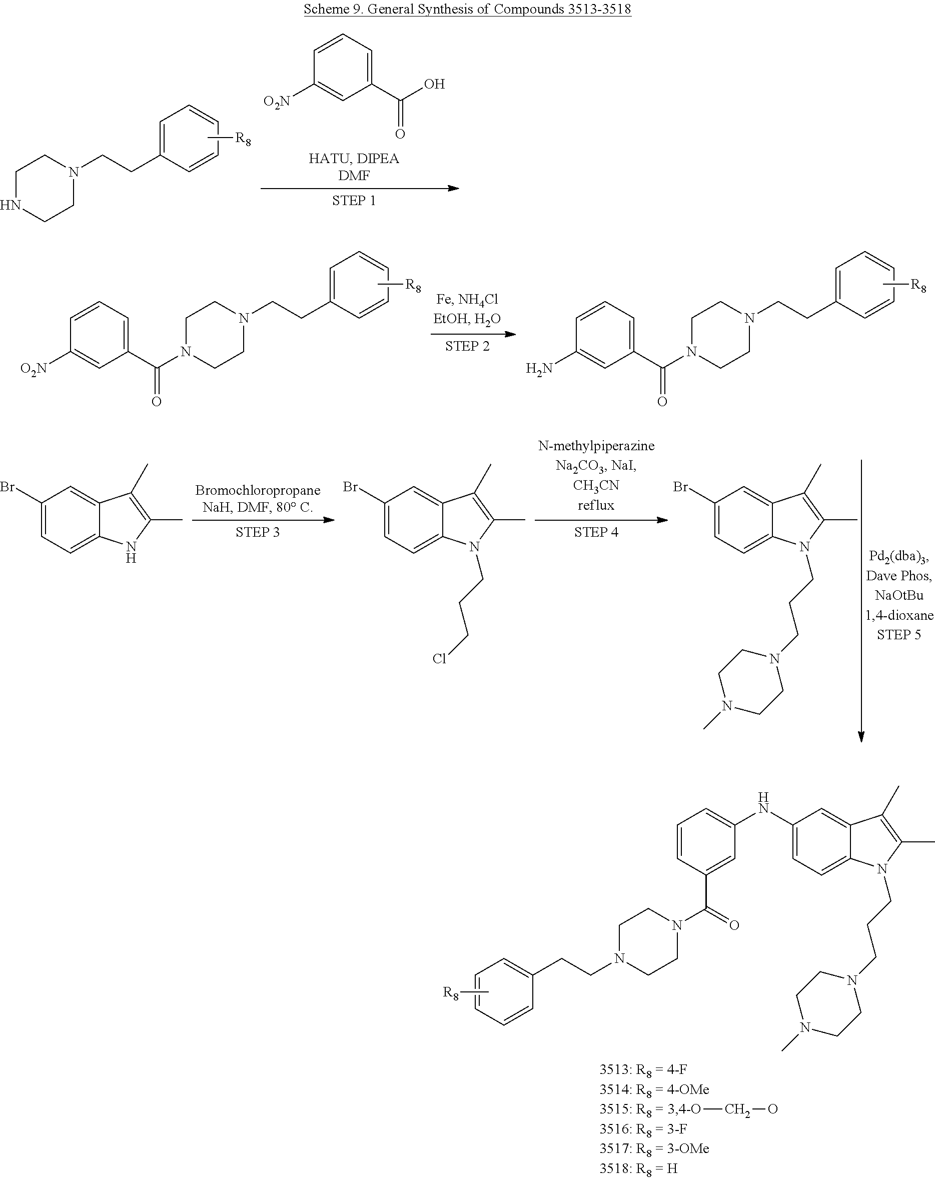

### Step 1: Preparation of (3-nitrophenyl) (4-phenethylpiperazin-1-yl)methanone

To a stirred solution of 3-nitrobenzoic acid (1.0 g, 5.9 mmol) in DMF (10 mL), DIPEA (1.97 mL, 11.3 mmol) was added. After stirring for 10 minutes, HATU (4.55 g, 11.97 mmol) was added and the mixture was stirred for a further 30 minutes at room temperature. The reaction mass was cooled to 0° C., 1-phenethylpiperazine (1.1 mL, 5.8 mmol) was added and the mixture was stirred at room temperature for 3 hours. The progress of the reaction was monitored by TLC. After complete consumption of the starting material, the reaction mixture was poured into ice water and extracted with ethyl acetate. The organic layer was washed with water and brine, dried over anhydrous Na₂SO₄ and concentrated. The crude compound was purified by flash column chromatography using 5% methanol-CH₂Cl₂ as an eluent to afford (3-nitrophenyl)(4-phenethylpiperazin-1-yl)methanone as a brown solid (1.5 g, 74%).

Other analogues prepared by this method:
(4-(4-fluorophenethyl)piperazin-1-yl)(3-nitrophenyl)methanone (94%)
(4-(4-methoxyphenethyl)piperazin-1-yl)(3-nitrophenyl)methanone (70%)
(4-(2-(benzo[d][1,3]dioxol-5-yl)ethyl)piperazin-1-yl)(3-nitrophenyl)methanone (31%)
(4-(3-fluorophenethyl)piperazin-1-yl)(3-nitrophenyl)methanone (64%)
(4-(3-methoxyphenethyl)piperazin-1-yl)(3-nitrophenyl)methanone (49%)

### Step 2: Preparation of (3-aminophenyl) (4-phenethylpiperazin-1-yl)methanone

To a stirred solution of (3-nitrophenyl)(4-phenethylpiperazin-1-yl)methanone (1.50 g, 4.42 mmol) in ethanol and water (1:1, 15 mL each) at room temperature, was added Fe powder (1.23 g, 22.1 mmol), and NH₄Cl (475 mg, 8.88 mmol). The reaction mixture was heated to 60° C. for 3 hours. After complete consumption of the starting material, the reaction mixture was filtered through Celite and the ethanol was evaporated. The aqueous layer was extracted with ethyl acetate and the organic layer was washed with brine, dried over anhydrous Na₂SO₄ and concentrated to give the crude product. The crude compound was purified by flash column chromatography using 6% methanol-CH₂Cl₂ as an eluent to afford (3-aminophenyl)(4-phenethylpiperazin-1-yl)methanone as a brown solid (1.0 g, 73%).

Other analogues prepared by this method:
(3-aminophenyl)(4-(4-fluorophenethyl)piperazin-1-yl)methanone (70%)
(3-aminophenyl)(4-(4-methoxyphenethyl)piperazin-1-yl)methanone (45%)
(3-aminophenyl)(4-(2-(benzo[d][1,3]dioxol-5-yl)ethyl)piperazin-1-yl)methanone (41%)
(3-aminophenyl)(4-(3-fluorophenethyl)piperazin-1-yl)methanone (89%)
(3-aminophenyl)(4-(3-methoxyphenethyl)piperazin-1-yl)methanone (52%)

### Step 3: Preparation of 5-bromo-1-(3-chloropropyl)-2,3-dimethyl-1H-indole

NaH (1.80 g, 44.6 mmol) was added portionwise to a stirred solution of 5-bromo-2,3-dimethyl-1H-indole (5.00 g, 22.3 mmol) in DMF (50 mL) at 0° C. The mixture was allowed to warm to room temperature for 30 minutes. To this, bromochloropropane (11.68 mL, 111.6 mmol) was added dropwise at 0° C. and the mixture was allowed to stir at room temperature for 3 hours. After complete consumption of the starting material, ice-cold water was added and the reaction mixture was extracted with ethyl acetate. The organic layer was washed with brine solution, dried over anhydrous Na₂SO₄ and concentrated under reduced pressure to give the crude product. The crude compound was purified by flash column chromatography using 5% ethyl acetate in pet-ether as an eluent to afford 5-bromo-1-(3-chloropropyl)-2,3-dimethyl-1H-indole as a pink solid (2.6 g, 40%). LCMS: m/z 302.10 [M+H]⁺.

### Step 4: Preparation of 5-bromo-2,3-dimethyl-1-(3-(4-methylpiperazin-1-yl)propyl)-1H-indole

To a stirred solution of 5-bromo-1-(3-chloropropyl)-2,3-dimethyl-1H-indole (9.00 g, 29.9 mmol) in acetonitrile (20 mL), sodium iodide (11.2 g, 74.7 mmol), sodium carbonate (7.93 g, 74.7 mmol) and then N-methylpiperazine (7.40 g, 74.7 mmol) were added at room temperature. The reaction mixture was heated to 75° C. for 16 hours. After complete consumption of the starting material, the reaction mixture was cooled to room temperature, diluted with ethyl acetate (60 mL), washed with water and brine solution, dried over anhydrous Na₂SO₄ and concentrated under reduced pressure to afford the crude product. The crude compound was purified by flash column chromatography using 5% methanol-CH₂Cl₂ as an eluent to afford 5-bromo-2,3-dimethyl-1-(3-(4-methylpiperazin-1-yl)propyl)-1H-indole as an off white solid (3.2 g, 30%). LCMS: m/z 365.98 [M+H]⁺.

### Step 5: Preparation of Compound 3513, (3-((2,3-dimethyl-1-(3-(4-methylpiperazin-1-yl)propyl)-1H-indol-5-yl)amino)phenyl) (4-(4-fluorophenethyl)piperazin-1-yl)methanone

(3-Aminophenyl)(4-(4-fluorophenethyl)piperazin-1-yl)methanone (120 mg, 0.355 mmol) and NaOtBu (78 mg, 0.82 mmol) were added to a stirred solution of 5-bromo-2,3-dimethyl-1-(3-(4-methylpiperazin-1-yl)propyl)-1H-indole (100 mg, 0.273 mmol) in 1,4-dioxane (3 mL). The reaction mixture was degassed using argon for 10 minutes. Pd₂(dba)₃ (17 mg, 0.019 mmol) and Dave-Phos (16 mg, 0.041 mmol) were added and the system was again degassed with argon for 10 minutes. The reaction mixture was heated to 90° C. for 16 hours. After complete consumption of the starting material, the reaction mixture was diluted with ethyl acetate and filtered through Celite. The organic layer was washed with water and brine solution, dried over anhydrous Na₂SO₄ and concentrated under reduced pressure to afford the crude product. The crude compound was purified by using prep-TLC, eluting with 5% methanol in CH₂Cl₂ to afford (3-((2,3-dimethyl-1-(3-(4-methylpiperazin-1-yl)propyl)-1H-indol-5-yl)amino)phenyl)(4-(4-fluorophenethyl)piperazin-1-yl)methanone (Compound 3513) as a pale yellow solid (50 mg, 15%).

¹H NMR (300 MHz, CD₃OD): δ 7.29 (d, J=8.4 Hz, 1H), 7.26-7.12 (m, 4H), 7.05-6.95 (m, 3H), 6.93 (dd, J=8.7 Hz, 1.8 Hz, 1H), 6.82 (br s, 1H), 6.67 (br d, J=7.2 Hz, 1H), 4.16 (t, J=6.6 Hz, 2H), 3.72 (br s, 2H), 3.51 (br s, 2H), 2.89-2.66 (m, 6H), 2.66-2.42 (m, 13H), 2.42-2.24 (m, 5H), 2.18 (s, 3H), 1.92 (quintet, J=6.9 Hz, 2H). LCMS: m/z 611 [M+H]⁺.

Other analogues prepared by this method:

Compound 3514, (3-((2,3-dimethyl-1-(3-(4-methylpiperazin-1-yl)propyl)-1H-indol-5-yl)amino)phenyl)(4-(4-methoxyphenethyl)piperazin-1-yl)methanone (43%).

¹H NMR (400 MHz, d₆-DMSO): δ 7.88 (br s, 1H), 7.32 (d, J=8.8 Hz, 1H), 7.16 (t, J=8.0 Hz, 1H), 7.13-7.09 (m, 3H), 6.91 (br d, J=8.0 Hz, 1H), 6.86 (dd, J=8.8 Hz, 2.0 Hz, 1H), 6.83 (d, J=8.4 Hz, 2H), 6.60 (d, J=7.6 Hz, 1H), 4.08 (t, J=6.8 Hz, 2H), 3.71 (s, 3H), 3.52 (br s, 4H), 2.66 (dd, J=8.8 Hz, 6.8 Hz, 2H), 2.46-2.11 (m, 22H), 1.78 (quintet, J=6.8 Hz, 2H). LCMS: m/z 623.17 [M+H]⁺.

Compound 3515, (4-(2-(benzo[d][1,3]dioxol-5-yl)ethyl)piperazin-1-yl)(3-((2,3-dimethyl-1-(3-(4-methylpiperazin-1-yl)propyl)-1H-indol-5-yl)amino)phenyl)methanone (28%).

¹H NMR (300 MHz, d₆-DMSO): δ 7.91 (br s, 1H), 7.32 (d, J=8.7 Hz, 1H), 7.17 (t, J=8.1 Hz, 1H), 7.13 (d, J=1.5 Hz, 1H), 6.91 (br d, J=8.4 Hz, 1H), 6.86 (dd, J=8.4 Hz, 1.5 Hz, 1H), 6.83-6.75 (m, 3H), 6.66 (dd, J=8.1 Hz, 1.5 Hz, 1H), 6.60 (br d, J=7.2 Hz, 1H), 5.95 (s, 2H), 4.08 (t, J=6.9 Hz, 2H), 3.51 (br s, 4H), 2.69-2.59 (m, 2H), 2.48-2.14 (m, 22H), 2.13 (s, 3H), 1.82-1.72 (m, 2H). LCMS: m/z 637.49 [M+H]⁺.

Compound 3516, (3-((2,3-dimethyl-1-(3-(4-methylpiperazin-1-yl)propyl)-1H-indol-5-yl)amino)phenyl)(4-(3-fluorophenethyl)piperazin-1-yl)methanone (22%).

¹H NMR (400 MHz, d₆-DMSO): δ 7.90 (br s, 1H), 7.35-7.27 (m, 2H), 7.17 (t, J=8.0 Hz, 1H), 7.13 (d, J=1.6 Hz, 1H), 7.11-7.04 (m, 2H), 7.00 (td, J=8.4 Hz, 2.0 Hz, 1H), 6.91 (br d, J=8.0 Hz, 1H), 6.87 (dd, J=8.4 Hz, 2.1 Hz, 1H), 6.80 (br s, 1H), 6.60 (br d, J=7.6 Hz, 1H), 4.08 (t, J=7.2 Hz, 2H), 3.51 (br s, 4H), 2.75 (dd, J=8.4 Hz, 7.2 Hz, 2H), 2.58-2.52 (m, 2H), 2.49-2.16 (m, 20H), 2.13 (s, 3H), 1.81-1.72 (m, 2H). LCMS: m/z 611.18 [M+H]⁺.

Compound 3517, (3-((2,3-dimethyl-1-(3-(4-methylpiperazin-1-yl)propyl)-1H-indol-5-yl)amino)phenyl)(4-(3-methoxyphenethyl)piperazin-1-yl)methanone (21%).

¹H NMR (400 MHz, d₆-DMSO): δ 7.91 (br s, 1H), 7.32 (d, J=8.4 Hz, 1H), 7.20-7.14 (m, 2H), 7.13 (d, J=2.0 Hz, 1H), 6.91 (dd, J=8.4 Hz, 1.2 Hz, 1H), 6.86 (dd, J=8.8 Hz, 2.0 Hz, 1H), 6.71-6.62 (m, 4H), 6.60 (br d, J=7.2 Hz, 1H), 4.08 (t, J=6.8 Hz, 2H), 3.73 (s, 3H), 3.54 (br s, 4H), 2.73-2.68 (m, 2H), 2.53-2.15 (m, 22H), 2.13 (s, 3H), 1.82-1.72 (m, 2H). LCMS: m/z 623.1 [M+H]⁺.

Compound 3518, (3-((2,3-dimethyl-1-(3-(4-methylpiperazin-1-yl)propyl)-1H-indol-5-yl)amino)phenyl)(4-phenethylpiperazin-1-yl)methanone (13%).

¹H NMR (400 MHz, d₆-DMSO): δ 7.90 (br s, 1H), 7.32 (d, J=8.4 Hz, 1H), 7.30-7.11 (m, 7H), 6.92 (br d, J=7.2 Hz, 1H), 6.87 (br s, J=8.4 Hz, 1H), 6.81 (br s, 1H), 6.60 (br d, J=7.2 Hz, 1H), 4.09 (t, J=6.8 Hz, 2H), 3.52 (br s, 4H), 2.75-2.68 (m, 2H), 2.63-2.14 (m, 22H), 2.13 (s, 3H), 1.84-1.73 (m, 2H). LCMS: m/z 593.55 [M+H]⁺.

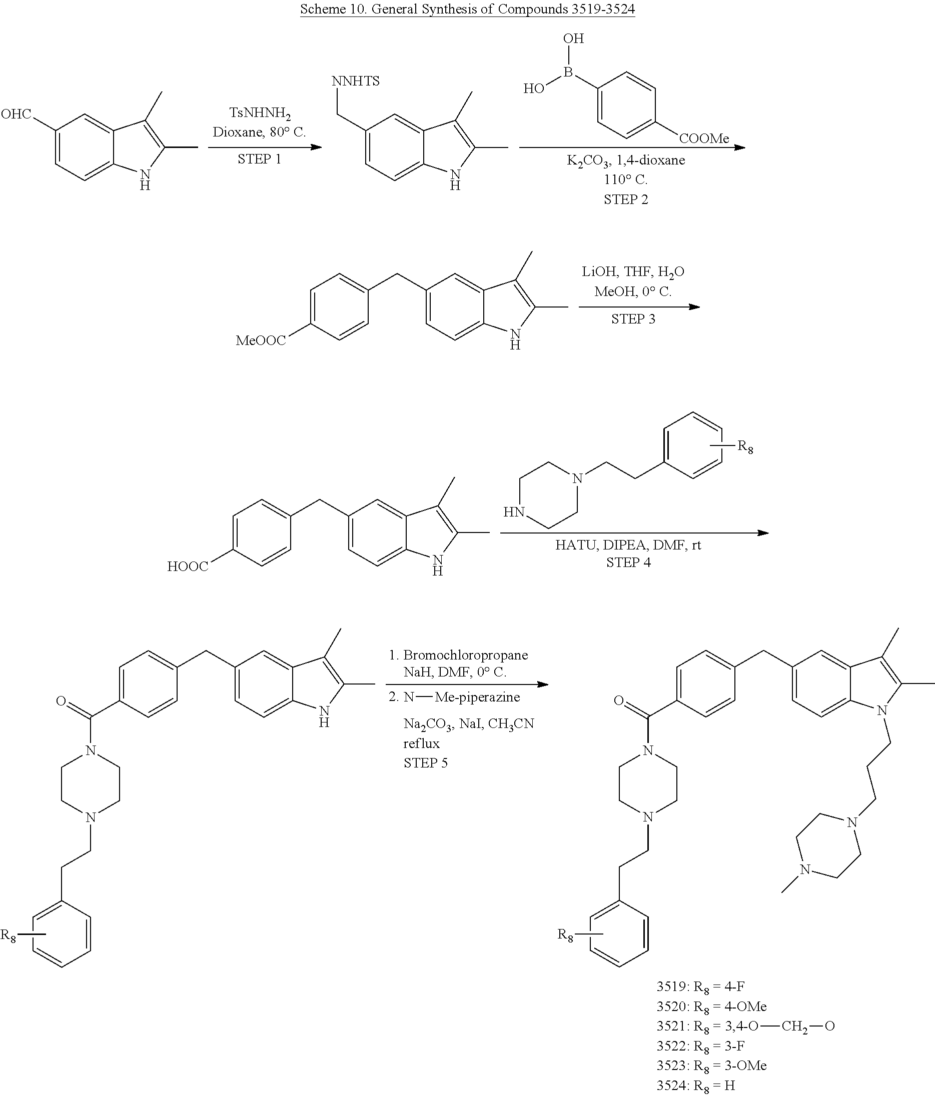

### Step 1 and 2: Preparation of methyl 4-((2,3-dimethyl-1H-indol-5-yl)methyl)benzoate

Tosyl hydrazine (2.14 g, 11.50 mmol) was added to a stirred solution of 2,3-dimethyl-1H-indole-5-carbaldehyde (2.0 g, 11.50 mmol) in dry 1, 4-dioxane (50 mL) at room temperature. The temperature was raised to 80° C. and maintained for 2 hours.

To the crude 2,3-dimethyl-5-((1-tosyl-2λ²-diazanyl)methyl)-1H-indole in the reaction mass, K₂CO₃ (2.38 g, 17.20 mmol) and (4-(methoxycarbonyl)phenyl)boronic acid (2.07 g, 11.50 mmol) were added at 80° C. The reaction temperature was raised to 110° C. and maintained for 4 hours. After complete consumption of the starting material, the reaction mass was concentrated, diluted with water and extracted with ethyl acetate. The combined organic layers were washed with water and brine, dried over anhydrous Na₂SO₄ and concentrated under reduced pressure to afford the crude product. The crude compound was purified by flash column chromatography using 20-25% ethyl acetate in petroleum ether as an eluent to obtain methyl 4-((2,3-dimethyl-1H-indol-5-yl)methyl)benzoate as a brown solid (1.5 g, 45%). LCMS: m/z 294.41 [M+H]⁺.

### Step 3: Preparation of 4-((2,3-dimethyl-1H-indol-5-yl)methyl)benzoic acid

To a solution of methyl 4-((2,3-dimethyl-1H-indol-5-yl)methyl)benzoate (3.0 g, 10.20 mmol) in THF:H₂O:MeOH (6:2:2) mixture was added LiOH.H₂O (1.28 g, 30.70 mmol) at 0° C. The reaction mixture was allowed to stir at room temperature for 16 hours. After complete consumption of the starting material, the reaction mass was concentrated and then partitioned between ethyl acetate and water. The aqueous layer was collected and acidified with saturated citric acid solution at 0° C. The precipitate thus obtained was collected by filtration and dried over vacuum to afford 4-((2,3-dimethyl-1H-indol-5-yl)methyl)benzoic acid as a brown solid (1.8 g, 63%). LCMS: m/z 280.39 [M+H]⁺.

### Step 4: Preparation of (4-((2,3-dimethyl-1H-indol-5-yl)methyl)phenyl)(4-phenethylpiperazin-1-yl)methanone

To a stirred solution of 4-((2, 3-dimethyl-1H-indol-5-yl)oxy)benzoic acid (250 mg, 0.896 mmol) in DMF (5 mL), DIPEA (0.5 mL) was added. After stirring for 10 minutes, HATU (511.0 mg, 1.3440 mmol) was added and the reaction mixture was stirred for 30 minutes. The reaction mass was cooled to 0° C., 1-phenethylpiperazine (187.5 mg, 0.9856 mmol) was added and the reaction mixture was stirred at room temperature overnight. After complete consumption of the starting material, the reaction mixture was poured into ice water. The precipitate thus obtained was collected by filtration and dried to give (4-((2,3-dimethyl-1H-indol-5-yl)methyl)phenyl)(4-phenethylpiperazin-1-yl)methanone as an off white solid (300 mg, 74%). LCMS: m/z 452.34 [M+H]⁺.

Other analogues prepared by this method
(4-((2,3-dimethyl-1H-indol-5-yl)methyl)phenyl)(4-(4-fluorophenethyl)piperazin-1-yl)methanone (74%). LCMS: m/z 470.1 [M+H]⁺.
(4-((2,3-dimethyl-1H-indol-5-yl)methyl)phenyl)(4-(4-methoxyphenethyl)piperazin-1-yl)methanone (70%). LCMS: m/z 482.0 [M+H]⁺.
(4-(2-(benzo[d][1,3]dioxol-5-yl)ethyl)piperazin-1-yl)(4-((2,3-dimethyl-1H-indol-5-yl)methyl)phenyl)methanone (79%). LCMS: m/z 496.0 [M+H]⁺.
(4-((2,3-dimethyl-1H-indol-5-yl)methyl)phenyl)(4-(3-fluorophenethyl)piperazin-1-yl)methanone (65%). LCMS: m/z 470.32 [M+H]⁺.
(4-((2,3-dimethyl-1H-indol-5-yl)methyl)phenyl)(4-(3-methoxyphenethyl)piperazin-1-yl)methanone (67%). LCMS: m/z 482.28 [M+H]⁺.

### Step 5-1: Preparation of (4-((1-(3-chloropropyl)-2,3-dimethyl-1H-indol-5-yl)methyl)phenyl) (4-phenethylpiperazin-1-yl) methanone

NaH (21.2 mg, 0.8857 mmol) was added portionwise to a stirred solution of (4-((2,3-dimethyl-1H-indol-5-yl)methyl)phenyl)(4-phenethylpiperazin-1-yl)methanone (200 mg, 0.4428 mmol) in DMF (4 mL) at 0° C. The mixture was allowed to warm to room temperature for 30 minutes. To this, bromochloropropane (0.10 mL, 0.8857 mmol) was added dropwise at 0° C. and the reaction mixture was allowed to stir at room temperature for 3 hours. After complete consumption of the starting material, ice-cold water was added into the reaction mixture, which was then extracted with ethyl acetate. The organic layer was washed with brine solution, dried over anhydrous Na₂SO₄ and concentrated under reduced pressure to afford the crude product. The crude compound was purified by flash column chromatography using ethyl acetate as an eluent to afford (4-((1-(3-chloropropyl)-2,3-dimethyl-1H-indol-5-yl)methyl)phenyl)(4-phenethylpiperazin-1-yl)methanone as a brown gummy solid (150 mg, 64%). LCMS: m/z 528.34 [M+H]⁺.

Other analogues prepared by this method:
4-((1-(3-chloropropyl)-2,3-dimethyl-1H-indol-5-yl)methyl)phenyl)(4-(4-fluorophenethyl)piperazin-1-yl)methanone (57%). LCMS: m/z 546.33 [M+H]⁺.
4-((1-(3-chloropropyl)-2,3-dimethyl-1H-indol-5-yl)methyl)phenyl)(4-(4-methoxyphenethyl)piperazin-1-yl)methanone (65%). LCMS: m/z 558.41 [M+H]⁺.
(4-(2-(benzo[d][1,3]dioxol-5-yl)ethyl)piperazin-1-yl)(4-((1-(3-chloropropyl)-2,3-dimethyl-1H-indol-5-yl)methyl)phenyl)methanone (43%). LCMS: m/z 572.34 [M+H]⁺.
4-((1-(3-chloropropyl)-2,3-dimethyl-1H-indol-5-yl)methyl)phenyl)(4-(3-fluorophenethyl)piperazin-1-yl)methanone (65%). LCMS: m/z 546.33 [M+H]⁺.
4-((1-(3-chloropropyl)-2,3-dimethyl-1H-indol-5-yl)methyl)phenyl)(4-(4-fluorophenethyl)piperazin-1-yl)methanone (78%). LCMS: m/z 558.36 [M+H]⁺.

### Step 5-2: Preparation of Compound 3524, (4-((2,3-dimethyl-1-(3-(4-methylpiperazin-1-yl)propyl)-1H-indol-5-yl)methyl)phenyl) (4-phenethylpiperazin-1-yl)methanone

To a stirred solution of (4-((1-(3-chloropropyl)-2,3-dimethyl-1H-indol-5-yl)methyl)phenyl)(4-phenethylpiperazin-1-yl)methanone (150 mg, 0.284 mmol) in acetonitrile (5 mL) at room temperature, sodium iodide (85.1 mg, 0.568 mmol) and sodium carbonate (90.3 mg, 0.852 mmol) were added, followed by N-methylpiperazine (71.1 mg, 0.710 mmol). The reaction mixture was heated to 75° C. for 16 hours. After complete consumption of the starting material, the reaction mixture was cooled to room temperature, diluted with ethyl acetate (40 mL), washed with water and brine solution, dried over anhydrous Na₂SO₄ and concentrated under reduced pressure to afford the crude product. The crude compound was purified by flash column chromatography using 5% methanol-CH₂Cl₂ as an eluent to afford (4-((2,3-dimethyl-1-(3-(4-methylpiperazin-1-yl)propyl)-1H-indol-5-yl)methyl)phenyl)(4-phenethylpiperazin-1-yl)methanone (Compound 3524) as an off-white solid (37 mg, 22%).

¹H NMR (300 MHz, d₆-DMSO): δ 7.36-7.12 (m, 11H), 6.91 (br d, J=8.7 Hz, 1H), 4.07 (t, J=7.2 Hz, 2H), 4.01 (s, 2H), 3.52 (br s, 4H), 2.75-2.69 (m, 2H), 2.61-2.56 (m, 2H), 2.47-2.15 (m, 20H), 2.14 (s, 3H), 1.82-1.69 (m, 2H). LCMS: m/z 592.58 [M+H]⁺.

Other analogues prepared by this method:

Compound 3519, (4-((2,3-dimethyl-1-(3-(4-methylpiperazin-1-yl)propyl)-1H-indol-5-yl)methyl)phenyl)(4-(4-fluorophenethyl)piperazin-1-yl)methanone (6%).

¹H NMR (300 MHz, CD₃OD): δ 7.36 (br s, 4H), 7.29-7.22 (m, 4H), 7.03 (t, J=9.0 Hz, 2H), 6.94 (br d, J=8.4 Hz, 1H), 4.19 (t, J=6.6 Hz, 2H), 4.08 (s, 2H), 3.71 (br s, 4H), 3.20-2.64 (m, 18H), 2.36 (br s, 6H), 2.19 (s, 3H), 1.99-1.88 (m, 2H). LCMS: m/z 610.56 [M+H]⁺.

Compound 3520, (4-((2,3-dimethyl-1-(3-(4-methylpiperazin-1-yl)propyl)-1H-indol-5-yl)methyl)phenyl)(4-(4-methoxyphenethyl)piperazin-1-yl)methanone (23%).

¹H NMR (300 MHz, CD₃OD): δ 7.32-7.23 (m, 6H), 7.12 (d, J=8.4 Hz, 2H), 6.92 (br d, J=8.7 Hz, 1H), 6.82 (d, J=8.4 Hz, 2H), 4.07 (t, J=6.6 Hz, 2H), 4.01 (br s, 2H), 3.70 (s, 3H), 3.52 (br s, 4H), 3.28-3.21 (m, 2H), 2.52-2.18 (m, 22H), 2.14 (s, 3H), 1.82-1.71 (m, 2H). LCMS: m/z 622.54 [M+H]⁺.

Compound 3521, (4-(2-(benzo[d][1,3]dioxol-5-yl)ethyl)piperazin-1-yl)(4-((2,3-dimethyl-1-(3-(4-methylpiperazin-1-yl)propyl)-1H-indol-5-yl)methyl)phenyl)methanone (18%).

¹H NMR (300 MHz, CD₃OD): δ 7.32 (br s, 4H), 7.27-7.20 (m, 2H), 6.93 (dd, J=9.0, 2.1 Hz, 1H), 6.73-6.63 (m, 3H), 5.88 (s, 2H), 4.17 (t, J=6.9 Hz, 2H), 4.07 (s, 2H), 3.73 (br s, 4H), 2.99-2.34 (m, 24H), 2.19 (s, 3H), 1.96-1.87 (m, 2H). LCMS: m/z 636.54 [M+H]⁺.

Compound 3522, (4-((2,3-dimethyl-1-(3-(4-methylpiperazin-1-yl)propyl)-1H-indol-5-yl)methyl)phenyl)(4-(3-fluorophenethyl)piperazin-1-yl)methanone (15%).

¹H NMR (400 MHz, CD₃OD): δ 7.35-7.23 (m, 7H), 7.11-7.04 (m, 2H), 7.04-6.95 (m, 1H), 6.91 (br d, J=8.4 Hz, 2H), 4.07 (t, J=6.9 Hz, 2H), 4.01 (s, 2H), 3.51 (br s, 4H), 2.79-2.68 (m, 2H), 2.57-2.16 (m, 22H), 2.14 (s, 3H), 1.82-1.71 (m, 2H). LCMS: m/z 610.53 [M+H]⁺.

Compound 3523, (4-((2,3-dimethyl-1-(3-(4-methylpiperazin-1-yl)propyl)-1H-indol-5-yl)methyl)phenyl)(4-(3-methoxyphenethyl)piperazin-1-yl)methanone (Compound 23) (20%).

¹H NMR (400 MHz, CD₃OD): δ 7.33-7.22 (m, 6H), 7.17 (t, J=8.1 Hz, 1H), 6.92 (br d, J=7.8 Hz, 1H), 6.81-6.72 (m, 3H), 4.16-4.01 (m, 4H), 3.76 (s, 3H), 3.53 (br s, 4H), 2.82-2.20 (m, 21H), 2.18 (s, 3H), 1.97 (s, 3H), 1.81-1.68 (m, 2H). LCMS: m/z 622.54 [M+H]⁺.

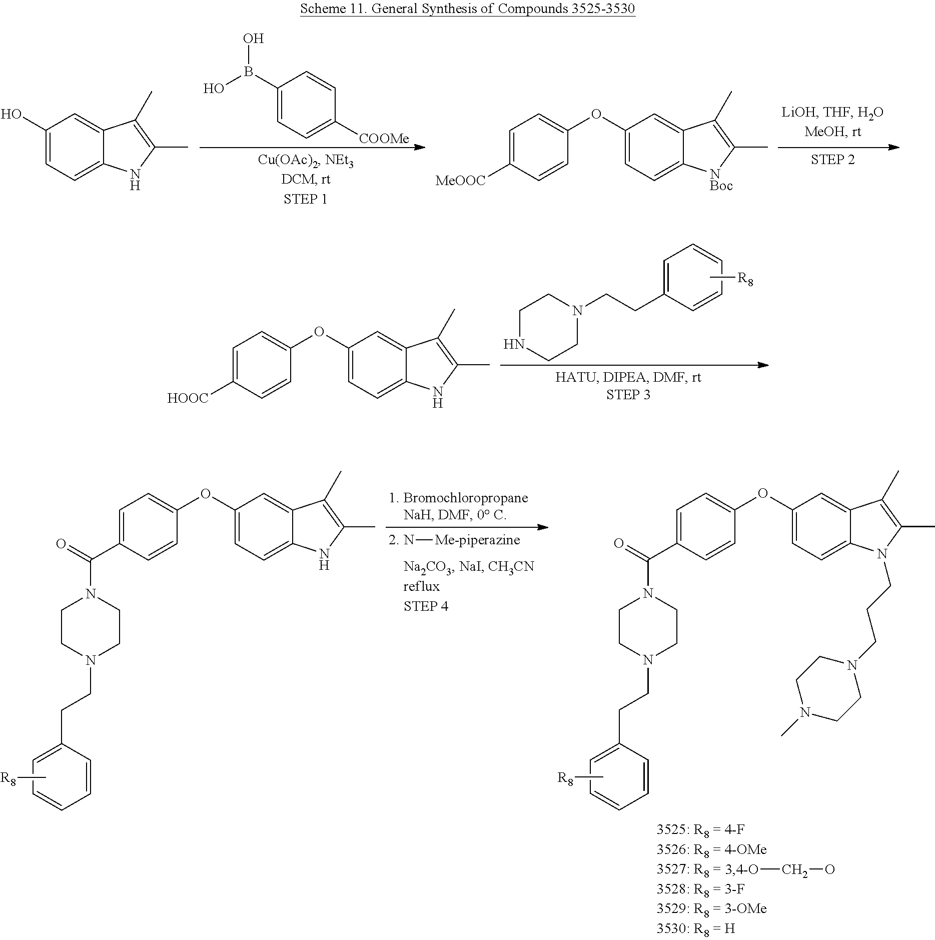

### Step 1: Preparation of tert-butyl 5-(4-(methoxycarbonyl)phenoxy)-2,3-dimethyl-1H-indole-1-carboxylate

To a stirred solution of tert-butyl 5-hydroxy-2,3-dimethyl-1H-indole-1-carboxylate (6.80 g, 26.0 mmol) in CH₂Cl₂ (70 mL) was added (4-(methoxycarbonyl)phenyl) boronic acid (14.0 g, 78.1 mmol), followed by Cu(OAc)₂ (11.8 g, 65.1 mmol) and TEA (34.0 mL, 260 mmol). The system was purged with oxygen gas for 4 hours. The whole reaction mass was stirred under an oxygen atmosphere overnight. After complete consumption of the starting material, the reaction mass was filtered through a Celite bed. The filtrate was diluted with water and extracted with CH₂Cl₂. The organic layer was washed with brine, dried over anhydrous Na₂SO₄ and concentrated under reduced pressure to afford the crude product. The crude compound was purified on 100-200 mesh silica gel eluting with 10% ethyl acetate in petroleum ether to obtain the desired product tert-butyl 5-(4-(methoxycarbonyl)phenoxy)-2,3-dimethyl-1H-indole-1-carboxylate as a brown liquid (6.50 g, 63%). LCMS: m/z 396.3 [M+H]⁺.

### Step 2: Preparation of 4-((2,3-dimethyl-1H-indol-5-yl)oxy)benzoic acid

To a stirred solution of tert-butyl 5-(4-(methoxycarbonyl)phenoxy)-2,3-dimethyl-1H-indole-1-carboxylate (6.50 g, 16.5 mmol) in THF (75 mL), water (75 mL), and methanol (75 mL) was added LiOH.H₂O (13.8 g, 329 mmol). The reaction mixture was stirred at room temperature for 4 hours. After complete consumption of the starting material, THF was evaporated under reduced pressure. The reaction mass was cooled to 0° C., acidified (pH 1) with 1 N HCl, then extracted with ethyl acetate. The organic layers were dried over anhydrous Na₂SO₄ and concentrated under reduced pressure to afford the crude product. Trituration with n-pentane to afforded pure 4-((2,3-dimethyl-1H-indol-5-yl)oxy)benzoic acid (4.00 g, 86%). LCMS: m/z 282.34 [M+H]⁺.

### Step 3: Preparation of (4-((2,3-dimethyl-1H-indol-5-yl)oxy)phenyl)(4-(4-fluorophenethyl)piperazin-1-yl)methanone

To a stirred solution of 4-((2,3-dimethyl-1H-indol-5-yl)oxy)benzoic acid (300 mg, 1.06 mmol) in DMF (3 mL), DIPEA (0.93 mL, 5.33 mmol) was added. After stirring for 10 minutes, HATU (0.6 g, 1.59 mmol) was added, followed by stirring for another 30 minutes at room temperature. The reaction mass was cooled to 0° C., 1-(4-fluorophenethyl)piperazine (0.40 g, 1.59 mmol) was added and the mixture was stirred at room temperature overnight. After complete consumption of the starting material, the reaction mixture was poured into ice water and extracted with ethyl acetate. The organic layer was washed with water and brine, dried over anhydrous Na₂SO₄ and concentrated under reduced pressure to afford the crude product. The crude compound was purified on 100-200 mesh silica eluting with 30% ethyl acetate in petroleum ether to obtain (4-((2,3-dimethyl-1H-indol-5-yl)oxy)phenyl)(4-(4-fluorophenethyl)piperazin-1-yl)methanone as a sticky brown solid (450 mg, 89%). LCMS: m/z 472.52 [M+H]⁺.

Other analogues prepared by this method:
(4-((2,3-dimethyl-1H-indol-5-yl)oxy)phenyl)(4-(4-methoxyphenethyl)piperazin-1-yl)methanone (77%). LCMS: m/z 484.50 [M+H]⁺.
((4-(2-(benzo[d][1,3]dioxol-5-yl)ethyl)piperazin-1-yl)(4-((2,3-dimethyl-1H-indol-5-yl)oxy)phenyl)methanone (87%). LCMS: m/z 498.56 [M+H]⁺.
(4-((2,3-dimethyl-1H-indol-5-yl)oxy)phenyl)(4-(3-fluorophenethyl)piperazin-1-yl)methanone (22%). LCMS: m/z 472.52 [M+H]⁺.
(4-((2,3-dimethyl-1H-indol-5-yl)oxy)phenyl)(4-(3-methoxyphenethyl)piperazin-1-yl)methanone (99%). LCMS: m/z 484.56 [M+H]⁺.
(4-((2,3-dimethyl-1H-indol-5-yl)oxy)phenyl)(4-phenethylpiperazin-1-yl)methanone (23%). LCMS: m/z 454.53 [M+H]⁺.

### Step 4-1: Preparation of (4-((1-(3-chloropropyl)-2,3-dimethyl-1H-indol-5-yl)oxy)phenyl) (4-(4-fluorophenethyl)piperazin-1-yl) methanone

NaH (75.0 mg, 1.88 mmol) was added portionwise to a stirred solution of (4-((2,3-dimethyl-1H-indol-5-yl)oxy)phenyl)(4-(4-fluorophenethyl)piperazin-1-yl)methanone (445 mg, 0.944 mmol) in DMF (5 mL) at 0° C. The mixture was allowed to warm to room temperature for 30 minutes. To this, bromochloropropane (0.19 mL, 1.88 mmol) was added dropwise at 0° C. The reaction mixture was stirred at room temperature for 3 hours. After complete consumption of the starting material, ice-cold water was added and the reaction mixture was extracted with ethyl acetate. The organic layer was washed with brine solution, dried over anhydrous Na₂SO₄ and concentrated under reduced pressure to afford the crude product. The crude compound was purified by flash column chromatography using 5% ethyl acetate in petroleum ether as an eluent to afford (4-((1-(3-chloropropyl)-2,3-dimethyl-1H-indol-5-yl)oxy)phenyl)(4-(4-fluorophenethyl)piperazin-1-yl)methanone as a brown gummy solid (340 mg, 66%).

LCMS: m/z 548.52 [M+H]⁺.

Other analogues prepared by this method:
(4-((1-(3-chloropropyl)-2,3-dimethyl-1H-indol-5-yl)oxy)phenyl)(4-(4-methoxyphenethyl)piperazin-1-yl)methanone (62%). LCMS: m/z 560.93 [M+H]⁺.
(4-(2-(benzo[d][1,3]dioxol-5-yl)ethyl)piperazin-1-yl)(4-((1-(3-chloropropyl)-2,3-dimethyl-1H-indol-5-yl)oxy)phenyl)methanone (61%). LCMS: m/z 574.92 [M+H]⁺.
(4-((1-(3-chloropropyl)-2,3-dimethyl-1H-indol-5-yl)oxy)phenyl)(4-(3-fluorophenethyl)piperazin-1-yl)methanone (62%). LCMS: m/z 548.32 [M+H]⁺.
(4-((1-(3-chloropropyl)-2,3-dimethyl-1H-indol-5-yl)oxy)phenyl)(4-(3-methoxyphenethyl)piperazin-1-yl)methanone (60%). LCMS: m/z 560.93 [M+H]⁺.
(4-((1-(3-chloropropyl)-2,3-dimethyl-1H-indol-5-yl)oxy)phenyl)(4-phenethylpiperazin-1-yl)methanone (67%). LCMS: m/z 530.57 [M+H]⁺.

### Step 4-2: Preparation of Compound 3525, (4-((2,3-dimethyl-1-(3-(4-methylpiperazin-1-yl)propyl)-1H-indol-5-yl)oxy)phenyl) (4-(4-fluorophenethyl)piperazin-1-yl)methanone

To a stirred solution of (4-((1-(3-chloropropyl)-2,3-dimethyl-1H-indol-5-yl)oxy)phenyl)(4-(4-fluorophenethyl)piperazin-1-yl)methanone (335 mg, 0.610 mmol) in acetonitrile (4 mL), were added sodium iodide (229 mg, 1.52 mmol) and sodium carbonate (194 mg, 1.83 mmol), followed by N-methylpiperazine (153 mg, 1.52 mmol) at room temperature. The reaction mixture was heated to 75° C. for 16 hours. After completion of starting material, the reaction mixture was cooled to room temperature, diluted with ethyl acetate (30 mL) and washed with water and brine, dried over anhydrous Na₂SO₄ and concentrated under reduced pressure to afford the crude product. The crude compound was purified by flash column chromatography using 5% methanol-CH₂Cl₂ as an eluent to afford (4-((2,3-dimethyl-1-(3-(4-methylpiperazin-1-yl)propyl)-1H-indol-5-yl)oxy)phenyl)(4-(4-fluorophenethyl)piperazin-1-yl)methanone (Compound 3525) as an off white solid (56 mg, 15%).

¹H NMR (400 MHz, CD₃OD) δ 7.38-7.34 (m, 3H), 7.22 (dd, J=8.4 Hz, 5.2 Hz, 2H), 7.09 (d, J=1.6 Hz, 1H), 6.98 (t, J=8.8 Hz, 2H), 6.94 (d, J=8.8 Hz, 2H), 6.80 (dd, J=8.8 Hz, 2.4 Hz, 1H), 4.20 (t, J=6.8 Hz, 2H), 3.63 (br s, 4H), 2.87-2.76 (m, 2H), 2.75-2.34 (m, 22H), 2.18 (s, 3H), 1.94 (quintet, J=6.8 Hz, 2H). LCMS: m/z 612.54 [M+H]⁺.

Other analogues prepared by this method:

Compound 3526, 4-((2,3-dimethyl-1-(3-(4-methylpiperazin-1-yl)propyl)-1H-indol-5-yl)oxy)phenyl)(4-(4-methoxyphenethyl)piperazin-1-yl)methanone (18%).

¹H NMR (300 MHz, CD₃OD) δ 7.38-7.33 (m, 3H), 7.12 (d, J=8.7 Hz, 2H), 7.09 (d, J=2.1 Hz, 1H), 6.94 (d, J=9.0 Hz, 2H), 6.86-6.77 (m, 3H), 4.19 (t, J=6.6 Hz, 2H), 3.75 (s, 3H), 3.63 (br s, 4H), 2.80-2.71 (m, 2H), 2.66-2.29 (m, 19H), 2.28 (s, 3H), 2.17 (s, 3H), 1.98-1.87 (m, 2H). LCMS: m/z 624.6 [M+H]⁺.

Compound 3527, (4-(2-(benzo[d][1,3]dioxol-5-yl)ethyl)piperazin-1-yl)(4-((2,3-dimethyl-1-(3-(4-methylpiperazin-1-yl)propyl)-1H-indol-5-yl)oxy)phenyl)methanone (9%).

¹H NMR (400 MHz, CD₃OD) δ 7.37 (d, J=8.4 Hz, 3H), 7.10 (d, J=2.0 Hz, 1H), 6.94 (d, J=8.4 Hz, 2H), 6.82 (dd, J=8.8 Hz, 2.0 Hz, 1H), 6.74-6.66 (m, 3H), 5.88 (s, 2H), 4.23 (t, J=6.8 Hz, 2H), 3.66 (br s, 4H), 2.95-2.40 (m, 21H), 2.39 (s, 3H), 2.18 (s, 3H), 1.99-1.91 (m, 2H). LCMS: m/z 638.52 [M+H].

Compound 3528, 4-((2,3-dimethyl-1-(3-(4-methylpiperazin-1-yl)propyl)-1H-indol-5-yl)oxy)phenyl)(4-(3-fluorophenethyl)piperazin-1-yl)methanone (25%).

¹H NMR (300 MHz, CD₃OD) δ 7.40-7.33 (m, 3H), 7.27 (td, J=8.1 Hz, 6.0 Hz, 1H), 7.09 (d, J=2.4 Hz, 1H), 7.07-6.85 (m, 5H), 6.81 (dd, J=8.7 Hz, 2.4 Hz, 1H), 4.20 (t, J=6.6 Hz, 2H), 3.64 (br s, 4H), 2.87-2.47 (m, 16H), 2.47-2.34 (m, 8H), 2.18 (s, 3H), 1.94 (quintet, J=7.2 Hz, 2H). LCMS: m/z 612.51 [M+H]⁺.

Compound 3529, 4-((2,3-dimethyl-1-(3-(4-methylpiperazin-1-yl)propyl)-1H-indol-5-yl)oxy)phenyl)(4-(3-methoxyphenethyl)piperazin-1-yl)methanone (26%).

¹H NMR (300 MHz, CD₃OD) δ 7.40-7.33 (m, 3H), 7.17 (t, J=8.1 Hz, 1H), 7.09 (d, J=1.8 Hz, 1H), 6.94 (d, J=8.7 Hz, 2H), 6.82-6.71 (m, 4H), 4.19 (t, J=7.2 Hz, 2H), 3.76 (s, 3H), 3.63 (br s, 4H), 2.86-2.49 (m, 16H), 2.43-2.32 (m, 5H), 2.32 (s, 3H), 2.18 (s, 3H), 2.00-1.88 (m, 2H). LCMS: m/z 624.52 [M+H]⁺.

Compound 3530, 4-((2,3-dimethyl-1-(3-(4-methylpiperazin-1-yl)propyl)-1H-indol-5-yl)oxy)phenyl)(4-phenethylpiperazin-1-yl)methanone (12%).

¹H NMR (400 MHz, CD₃OD) δ 7.37 (d, J=8.4 Hz, 3H), 7.29-7.14 (m, 5H), 7.09 (d, J=2.0 Hz, 1H), 6.94 (d, J=8.4 Hz, 2H), 6.81 (dd, J=9.2 Hz, 2.4 Hz, 1H), 4.21 (t, J=7.2 Hz, 2H), 3.63 (br s, 4H), 2.99-2.72 (m, 6H), 2.72-2.46 (m, 13H), 2.46-2.32 (m, 5H), 2.18 (s, 3H), 1.95 (quintet, J=6.8 Hz 2H). LCMS: m/z 594.52 [M+H]⁺.

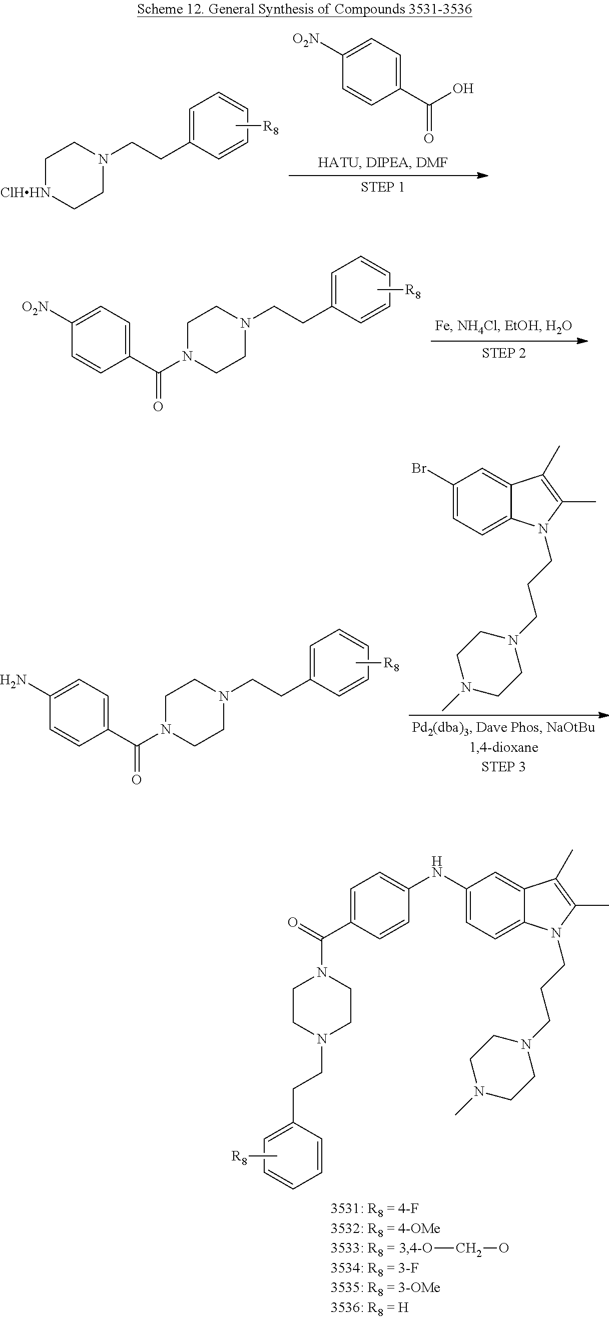

### Step 1: Preparation of (4-(3-methoxyphenethyl)piperazin-1-yl) (4-nitrophenyl)methanone

To a stirred solution of 1-(3-methoxyphenethyl)piperazine hydrochloride (500 mg, 2.99 mmol) in DMF (10 mL), HATU (2.27 g, 5.98 mmol), DIPEA (2.47 mL, 14.9 mmol) and 4-nitrobenzoic acid (930 mg, 3.59 mmol) were added at room temperature. The reaction mixture was stirred at room temperature for 16 hours. After complete consumption of the starting material, the reaction mixture was taken in ice water and extracted with ethyl acetate. The organic layer was washed with water and brine, dried over anhydrous Na₂SO₄ and concentrated. The crude product was purified by flash column chromatography using 5% methanol-CH₂Cl₂ as an eluent to afford (4-(3-methoxyphenethyl)piperazin-1-yl)(4-nitrophenyl)methanone as a brown solid (1.0 g, 90%). LCMS: m/z 370.0 [M+H]⁺.

Other analogues prepared by this method:
(4-(4-fluorophenethyl)piperazin-1-yl)(4-nitrophenyl)methanone (100%)
(4-(4-methoxyphenethyl)piperazin-1-yl)(4-nitrophenyl)methanone (72%)
(4-(2-(benzo[d][1,3]dioxol-5-yl)ethyl)piperazin-1-yl)(4-nitrophenyl)methanone (27%)
(4-(3-fluorophenethyl)piperazin-1-yl)(4-nitrophenyl)methanone (68%)
(4-nitrophenyl)(4-phenethylpiperazin-1-yl)methanone (75%)

### Step 2: Preparation of (4-aminophenyl) (4-(3-methoxyphenethyl)piperazin-1-yl)methanone

To a stirred solution of (4-(3-methoxyphenethyl)piperazin-1-yl)(4-nitrophenyl)methanone (900 mg, 2.43 mmol) in ethanol and water (1:1, 10 mL each) at room temperature, was added Fe powder (300 mg, 12.16 mmol), and NH₄Cl (325 mg, 6.08 mmol). The reaction mixture was heated to 60° C. for 3 hours. After complete consumption of the starting material, the reaction mixture was filtered through Celite and the ethanol was evaporated. The aqueous layer was extracted with Ethyl acetate and the Ethyl acetate layer was washed with brine, dried over anhydrous Na₂SO₄ and concentrated to give the crude product. The crude compound was purified by flash column using 6% Methanol-DCM as an eluent to afford (4-aminophenyl)(4-(3-methoxyphenethyl)piperazin-1-yl)methanone as a brown solid (300 mg, 41%). LCMS: m/z 340.0 [M+H]⁺.

Other analogues prepared by this method:
(4-aminophenyl)(4-(4-fluorophenethyl)piperazin-1-yl)methanone (72%)
(4-aminophenyl)(4-(4-methoxyphenethyl)piperazin-1-yl)methanone (42%)
(4-aminophenyl)(4-(2-(benzo[d][1,3]dioxol-5-yl)ethyl)piperazin-1-yl)methanone (43%)
(4-aminophenyl)(4-(3-fluorophenethyl)piperazin-1-yl)methanone (100%)
(4-aminophenyl)(4-phenethylpiperazin-1-yl)methanone (74%)

### Step 3: Preparation of Compound 3531, (4-((2,3-dimethyl-1-(3-(4-methylpiperazin-1-yl)propyl)-1H-indol-5-yl)amino)phenyl) (4-(4-fluorophenethyl)piperazin-1-yl) methanone

(4-Aminophenyl)(4-(4-fluorophenethyl)piperazin-1-yl)methanone (120 mg, 0.355 mmol) and NaOtBu (78 mg, 0.82 mmol) were added to a stirred solution of 5-bromo-2,3-dimethyl-1-(3-(4-methylpiperazin-1-yl)propyl)-1H-indole (100 mg, 0.273 mmol) in 1,4-dioxane (3 mL). The reaction mixture was degassed using argon for 10 minutes. Pd₂(dba)₃ (17 mg, 0.019 mmol) and Dave-Phos (16 mg, 0.041 mmol) were added and the mixture was again degassed using argon for 10 minutes. The reaction mixture was heated to 90° C. for 16 hours. After complete consumption of the starting material, the reaction mixture was diluted using Ethyl acetate and filtered through Celite. The organic layer was washed with water and brine solution, dried over anhydrous Na₂SO₄ and concentrated under reduced pressure to afford the crude product. The crude compound was purified by using preparative TLC, eluting with 5% methanol in CH₂Cl₂ to afford (4-((2,3-dimethyl-1-(3-(4-methylpiperazin-1-yl)propyl)-1H-indol-5-yl)amino)phenyl)(4-(4-fluorophenethyl)piperazin-1-yl)methanone (Compound 3531) as a pale yellow solid (20 mg, 21%).

¹H NMR (300 MHz, d₆-DMSO): δ 8.11 (br s, 1H), 7.33 (d, J=8.4 Hz, 1H), 7.26 (dd, J=8.4 Hz, 5.7 Hz, 2H), 7.20 (d, J=8.4 Hz, 2H), 7.15 (br s, 1H), 7.09 (t, J=9.0 Hz, 2H), 6.92-6.83 (m, 3H), 4.10 (t, J=6.9 Hz, 2H), 3.51 (br s, 4H), 2.78-2.70 (m, 2H), 2.64-2.15 (m, 22H), 2.14 (s, 3H), 1.85-1.76 (m, 2H). LCMS: m/z 611.53 [M+H]⁺.

Other analogues prepared by this method:

Compound 3532, (4-((2,3-dimethyl-1-(3-(4-methylpiperazin-1-yl)propyl)-1H-indol-5-yl)amino)phenyl)(4-(4-methoxyphenethyl)piperazin-1-yl)methanone (42%).

¹H NMR (300 MHz, d₆-DMSO): δ 8.12 (br s, 1H), 7.33 (d, J=8.4 Hz, 1H), 7.20 (d, J=8.4 Hz, 2H), 7.17-7.10 (m, 3H), 6.92-6.80 (m, 5H), 4.10 (t, J=6.6 Hz, 2H), 3.71 (s, 3H), 3.50 (br s, 4H), 2.77-2.56 (m, 6H), 2.48-2.19 (m, 18H), 2.14 (s, 3H), 1.86-1.75 (m, 2H). LCMS: m/z 623.51 [M+H]⁺.

Compound 3533, (4-(2-(benzo[d][1,3]dioxol-5-yl)ethyl)piperazin-1-yl)(4-((2,3-dimethyl-1-(3-(4-methylpiperazin-1-yl)propyl)-1H-indol-5-yl)amino)phenyl)methanone (18%).

¹H NMR (400 MHz, d₆-DMSO): δ 8.12 (br s, 1H), 7.33 (d, J=8.8 Hz, 1H), 7.20 (d, J=8.4 Hz, 2H), 7.15 (d, J=1.6 Hz, 1H), 6.92-6.76 (m, 5H), 6.67 (br d, J=8.4 Hz, 1H), 5.95 (s, 2H), 4.09 (t, J=6.8 Hz, 2H), 3.50 (br s, 4H), 2.69-2.61 (m, 2H), 2.47-2.17 (m, 22H), 2.14 (s, 3H), 1.85-1.73 (m, 2H). LCMS: m/z 637.47 [M+H]⁺.

Compound 3534, (4-((2,3-dimethyl-1-(3-(4-methylpiperazin-1-yl)propyl)-1H-indol-5-yl)amino)phenyl)(4-(3-fluorophenethyl)piperazin-1-yl)methanone (11%).

¹H NMR (300 MHz, d₆-DMSO): δ 8.11 (br s, 1H), 7.37-7.26 (m, 2H), 7.20 (d, J=8.4 Hz, 2H), 7.15 (d, J=1.8 Hz, 1H), 7.13-7.05 (m, 2H), 7/00 (td, J=8.7 Hz, 2.1 Hz, 1H), 6.93-6.79 (m, 3H), 4.09 (t, J=6.9 Hz, 2H), 3.50 (br s, 4H), 2.81-2.72 (m, 2H), 2.61-2.53 (m, 2H), 2.48-2.29 (m, 15H), 2.27-2.10 (m, 8H), 1.84-1.72 (m, 2H). LCMS: m/z 611.53 [M+H]⁺.

Compound 3535, (4-((2,3-dimethyl-1-(3-(4-methylpiperazin-1-yl)propyl)-1H-indol-5-yl)amino)phenyl)(4-(3-methoxyphenethyl)piperazin-1-yl)methanone (30%).

¹H NMR (300 MHz, CD₃OD): δ 7.90 (br s, 1H), 7.35-7.12 (m, 5H), 7.00-6.85 (m, 3H), 6.83-6.72 (m, 3H), 4.20 (t, J=6.6 Hz, 2H), 3.77 (s, 3H), 3.71 (br s, 3H), 3.15-2.89 (m, 4H), 2.89-2.41 (m, 17H), 2.37 (s, 3H), 2.19 (s, 3H), 2.03-1.89 (m, 2H). LCMS: m/z 623.51 [M+H]⁺.

Compound 3536, (4-((2,3-dimethyl-1-(3-(4-methylpiperazin-1-yl)propyl)-1H-indol-5-yl)amino)phenyl)(4-(4-methoxyphenethyl)piperazin-1-yl)methanone (10%).

¹H NMR (400 MHz, d₆-DMSO): δ 8.11 (br s, 1H), 7.41-7.09 (m, 9H), 6.96-6.77 (m, 3H), 4.09 (br s, 2H), 3.50 (br s, 4H), 2.78-2.70 (m, 2H), 2.63-2.50 (m, 9H), 2.39-2.09 (m, 16H), 1.83-1.74 (m, 2H). LCMS: m/z 593.55 [M+H]⁺.

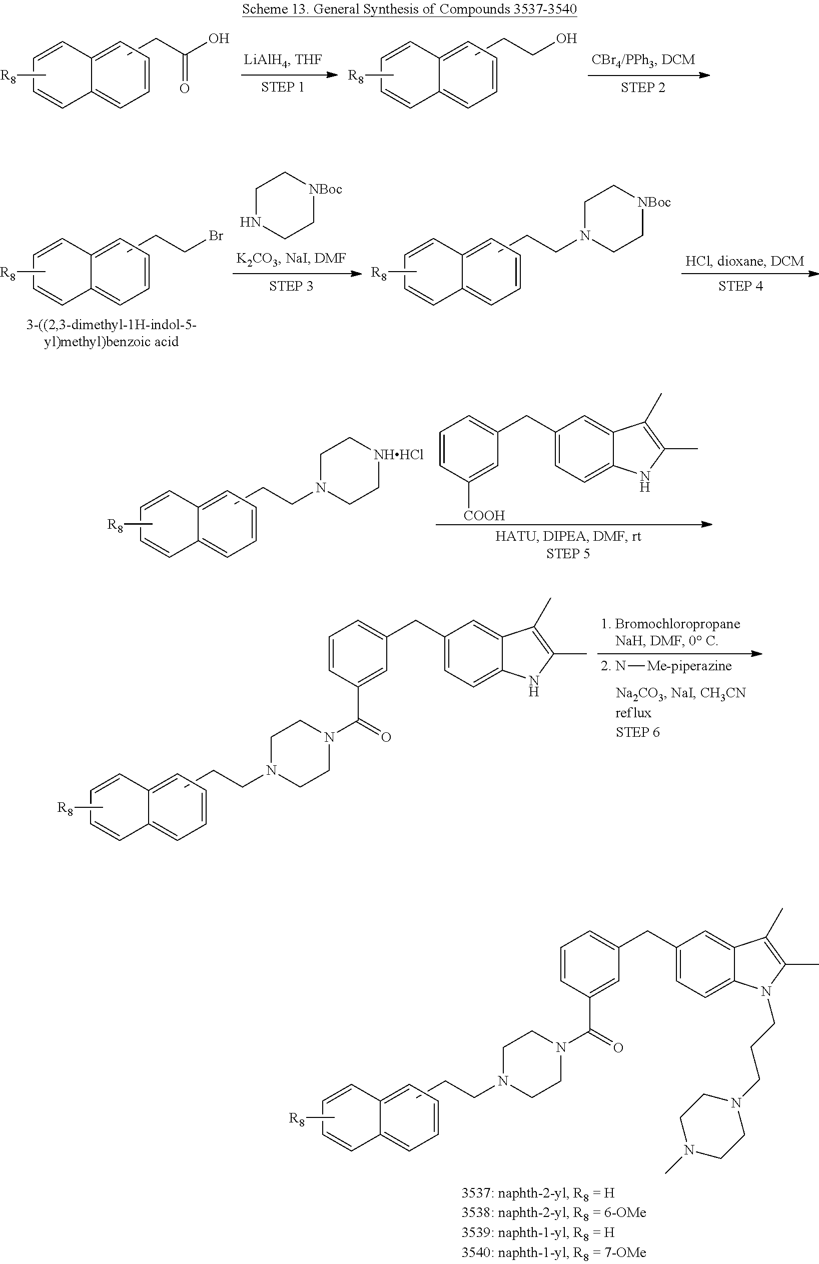

### Step 1: Preparation of 2-(6-methoxynaphthalen-2-yl)ethan-1-ol

To a mixture of LiAlH₄ (789 mg, 13.9 mmol) in THF (100 mL) at 0° C. was added 2-(6-methoxynaphthalen-2-yl)acetic acid (3.00 g, 13.9 mmol). The mixture was stirred for 10 minutes at this temperature, after which the reaction mass was slowly warmed to room temperature and stirred for 2 hours. After complete consumption of the starting material as determined by TLC, the reaction mixture was quenched with ethyl acetate (3 mL) and saturated ammonium chloride solution (20 mL) at 0° C., filtered and concentrated to give 2-(6-methoxynaphthalen-2-yl)ethan-1-ol as a white solid (2.5 g, 95%).

¹H NMR (300 MHz, d₆-DMSO): δ 7.89-7.80 (m, 3H), 7.71 (br s, 1H), 7.51-7.38 (m, 3H), 4.67 (t, J=5.1 Hz, 1H), 3.70 (q, J=6.9 Hz, 2H), 2.89 (t, J=6.9 Hz, 2H).

Other analogues prepared by this method:
2-(naphthalen-2-yl)ethan-1-ol (77%)
2-(naphthalen-1-yl)ethan-1-ol (86%)
2-(7-methoxynaphthalen-1-yl)ethan-1-ol (85%)

### Step 2: Preparation of 2-(2-bromoethyl)naphthalene

To a solution of 2-(naphthalen-2-yl)ethan-1-ol (5.20 g, 30.2 mmol) in DCM (100 mL), CBr₄ (10.99 g, 33.21 mmol) and PPh₃ (8.70 g, 33.2 mmol) were added portionwise at 0° C. The reaction mixture was stirred for 10 minutes, then the temperature was raised to room temperature and maintained for 2 hours. After complete consumption of the starting material as determined by TLC, ice-cold water was added into the reaction mixture, which was then extracted with DCM. The combined organic layers were washed with water, then brine solution, dried over anhydrous Na₂SO₄ and concentrated under reduced pressure to afford the crude product. The crude compound was purified by flash column chromatography using 10-15% Ethyl acetate in petroleum ether as an eluent, to give 2-(2-bromoethyl)naphthalene as a yellow liquid (5.2 g, 71%).

Other analogues prepared by this method:
2-(2-bromoethyl)-6-methoxynaphthalene (79%)
1-(2-bromoethyl)naphthalene (73%)
1-(2-bromoethyl)-7-methoxynaphthalene (78%)

### Step 3: Preparation of tert-butyl 4-(2-(naphthalen-2-yl)ethyl)piperazine-1-carboxylate

To a solution of N-Boc piperazine (4.94 g, 26.6 mmol) in DMF (60 mL), were added K₂CO₃ (6.11 g, 44.3 mmol), 2-(2-bromoethyl)naphthalene (5.2 g, 22.1 mmol) and NaI (3.31 g, 22.1 mmol) at room temperature. The reaction mixture was heated to 80° C. for 12 hours. After complete consumption of the starting material as determined by TLC, ice-cold water was added into the reaction mixture, which was then extracted with ethyl acetate. The combined organic layers were washed with water and brine, dried over anhydrous Na₂SO₄ and concentrated under reduced pressure to afford the crude product. The crude compound was purified by flash column chromatography using 40-50% Ethyl acetate in petroleum ether as an eluent to give tert-butyl 4-(2-(naphthalen-2-yl)ethyl)piperazine-1-carboxylate as a brown gummy solid (7.0 g, 93%). LCMS: m/z 341.35 [M+H]⁺.

Other analogues prepared by this method:
tert-butyl 4-(2-(6-methoxynaphthalen-2-yl)ethyl)piperazine-1-carboxylate (58%)
tert-butyl 4-(2-(naphthalen-1-yl)ethyl)piperazine-1-carboxylate (46%)
tert-butyl 4-(2-(7-methoxynaphthalen-1-yl)ethyl)piperazine-1-carboxylate (74%)
tert-butyl 4-(2-(quinolin-6-yl)ethyl)piperazine-1-carboxylate

### Step 4: Preparation of 1-(2-(naphthalen-2-yl)ethyl)piperazine hydrochloride

To a stirred solution of tert-butyl 4-(2-(naphthalen-2-yl)ethyl)piperazine-1-carboxylate (7.00 g, 20.6 mmol) in DCM (70 mL) was added 1,4-dioxane/HCl (10.3 mL, ˜4 M) at 0° C. The reaction mixture was stirred for 10 minutes, then the temperature was raised to room temperature, which was maintained for 12 hours. After complete consumption of the starting material as determined by TLC, the reaction mass was concentrated, diethyl ether was added and the mixture was filtered to afford 1-(2-(naphthalen-2-yl)ethyl)piperazine hydrochloride as a brown solid (6.0 g, 89%). LCMS: m/z 241.29 [(M+HCl)+H]⁺.

Other analogues prepared by this method:
1-(2-(6-methoxynaphthalen-2-yl)ethyl)piperazine hydrochloride (91%)
1-(2-(naphthalen-1-yl)ethyl)piperazine hydrochloride (68%)
1-(2-(7-methoxynaphthalen-1-yl)ethyl)piperazine hydrochloride (74%)

### Step 5: Preparation of (3-((2,3-dimethyl-1H-indol-5-yl)methyl)phenyl)(4-(2-(naphthalen-2-yl)ethyl)piperazin-1-yl)methanone

HATU (511.0 mg, 1.344 mmol) was added to a stirred solution of 3-((2,3-dimethyl-1H-indol-5-yl)methyl)benzoic acid (250 mg, 0.896 mmol) and DIPEA (0.5 mL) in DMF (5 mL) at 0° C. The reaction mixture was stirred for 30 minutes at room temperature. Then 1-(2-(naphthalen-2-yl)ethyl)piperazine hydrochloride (273 mg, 0.986 mmol) was added and the mixture was stirred at room temperature overnight. The progress of the reaction was monitored by TLC. After complete consumption of the starting material, the reaction mixture was poured into ice water. The resulting precipitate was collected by filtration and dried. The crude compound was purified by flash column chromatography using 5% methanol in CH₂Cl₂ as an eluent to obtain (3-((2,3-dimethyl-1H-indol-5-yl)methyl)phenyl)(4-(2-(naphthalen-2-yl)ethyl)piperazin-1-yl)methanone as a brown solid (0.40 g, 86%). LCMS: m/z 517.55 [M+H]⁺.

Other analogues prepared by this method:
(3-((2,3-dimethyl-1H-indol-5-yl)methyl)phenyl)(4-(2-(6-methoxynaphthalen-2-yl)ethyl)piperazin-1-yl)methanone (63%)
(3-((2,3-dimethyl-1H-indol-5-yl)methyl)phenyl)(4-(2-(naphthalen-1-yl)ethyl)piperazin-1-yl)methanone (63%)
(3-((2,3-dimethyl-1H-indol-5-yl)methyl)phenyl)(4-(2-(7-methoxynaphthalen-1-yl)ethyl)piperazin-1-yl)methanone (61%)

### Step 6-1: Preparation of (3-((1-(3-chloropropyl)-2,3-dimethyl-1H-indol-5-yl)methyl)phenyl) (4-(2-(6-methoxynaphthalen-2-yl)ethyl)piperazin-1-yl)methanone

NaH (102 mg, 2.56 mmol) was added portionwise to a stirred solution of (3-((2,3-dimethyl-1H-indol-5-yl)methyl)phenyl)(4-(2-(6-methoxynaphthalen-2-yl)ethyl)piperazin-1-yl)methanone (680 mg, 1.22 mmol) in DMF (7 mL) at 0° C. The mixture was allowed to warm to room temperature for 30 minutes. Bromochloropropane (1.20 mL, 6.49 mmol) was added dropwise at 0° C. and the reaction mixture was allowed to stir at room temperature for 3 hours. After complete consumption of the starting material, ice-cold water was added into reaction mixture, which was then extracted with ethyl acetate. The organic layer was washed with brine solution, dried over anhydrous Na₂SO₄ and concentrated under reduced pressure to afford the crude product. The crude compound was purified by flash column chromatography using ethyl acetate as an eluent to afford (3-((1-(3-chloropropyl)-2,3-dimethyl-1H-indol-5-yl)methyl)phenyl)(4-(2-(6-methoxynaphthalen-2-yl)ethyl)piperazin-1-yl)methanone as a brown gummy solid (250 mg, 42%). LCMS: m/z 608.3 [M+H]⁺.

Other analogues prepared by this method:
(3-((1-(3-chloropropyl)-2,3-dimethyl-1H-indol-5-yl)methyl)phenyl)(4-(2-(naphthalen-2-yl)ethyl)piperazin-1-yl)methanone (98%)
(3-((1-(3-chloropropyl)-2,3-dimethyl-1H-indol-5-yl)methyl)phenyl)(4-(2-(naphthalen-1-yl)ethyl)piperazin-1-yl)methanone (66%)
(3-((1-(3-chloropropyl)-2,3-dimethyl-1H-indol-5-yl)methyl)phenyl)(4-(2-(7-methoxynaphthalen-1-yl)ethyl)piperazin-1-yl)methanone (44%)

### Step 6-2: Preparation of Compound 3537, (3-((2,3-dimethyl-1-(3-(4-methylpiperazin-1-yl)propyl)-1H-indol-5-yl)methyl)phenyl) (4-(2-(naphthalen-2-yl)ethyl)piperazin-1-yl)methanone

To a stirred solution of (3-((1-(3-chloropropyl)-2,3-dimethyl-1H-indol-5-yl)methyl)phenyl)(4-(2-(naphthalen-2-yl)ethyl)piperazin-1-yl)methanone (150 mg, 0.284 mmol) in acetonitrile (5 mL) were added sodium iodide (85.09 mg, 0.5680 mmol) and sodium carbonate (90.31 mg, 0.8520 mmol) followed by N-methylpiperazine (71.10 mg, 0.7100 mmol) at room temperature. The reaction mixture was heated to 75° C. for 16 hours. After complete consumption of the starting material, the reaction mixture was cooled to room temperature and diluted with ethyl acetate (40 mL). The mixture was washed with water and brine solution, dried over anhydrous Na₂SO₄ and concentrated under reduced pressure. The crude compound was purified by flash column chromatography using 5% methanol-CH₂Cl₂ as an eluent to afford (3-((2,3-dimethyl-1-(3-(4-methylpiperazin-1-yl)propyl)-1H-indol-5-yl)methyl)phenyl)(4-(2-(naphthalen-2-yl)ethyl)piperazin-1-yl)methanone (Compound 3537) as a pale yellow solid (35 mg, 11%).

¹H NMR (300 MHz, CD₃OD): δ 8.05 (br d, J=8.1 Hz, 1H), 7.88 (dd, J=7.5 Hz, 1.5 Hz, 1H), 7.75 (br d, J=8.1 Hz, 1H), 7.57-7.32 (m, 6H), 7.34-7.18 (m, 3H), 7.09 (br s, 1H), 6.90 (dd, J=8.1 Hz, 1.5 Hz, 1H), 4.08 (s, 2H), 3.93 (t, J=6.9 Hz, 2H), 3.73 (br s, 2H), 3.28-3.20 (m, 4H), 2.68-2.10 (m, 25H), 1.77 (quintet, J=6.9 Hz, 2H). LCMS: m/z 642.54 [M+H]⁺.

Other analogues prepared by this method:

Compound 3538, (3-((2,3-dimethyl-1-(3-(4-methylpiperazin-1-yl)propyl)-1H-indol-5-yl)methyl)phenyl)(4-(2-(6-methoxynaphthalen-2-yl)ethyl)piperazin-1-yl)methanone (23%).

¹H NMR (300 MHz, d₆-DMSO): δ 7.84 (d, J=8.7 Hz, 1H), 7.70 (br d, J=7.5 Hz, 1H), 7.38-7.14 (m, 10H), 6.90 (br d, J=8.4 Hz, 1H), 4.07-3.98 (m, 4H), 3.89 (s, 3H), 3.61 (br s, 2H), 3.33 (br s, 2H), 3.21-3.12 (m, 2H), 2.65-2.11 (m, 25H), 1.76-1.64 (m, 2H). LCMS: m/z 672.57 [M+H]⁺.

Compound 3539, (3-((2,3-dimethyl-1-(3-(4-methylpiperazin-1-yl)propyl)-1H-indol-5-yl)methyl)phenyl)(4-(2-(naphthalen-1-yl)ethyl)piperazin-1-yl)methanone (22%).

¹H NMR (300 MHz, CD₃OD): δ 8.05 (br d, J=8.1 Hz, 1H), 7.88 (br d, J=7.2 Hz, 1H), 7.76 (br d, J=7.8 Hz, 1H), 7.58-7.33 (m, 6H), 7.26-7.15 (m, 3H), 7.09 (br s, 1H), 6.91 (br d, J=8.1 Hz, 1H), 4.09 (s, 2H), 3.93 (t, J=6.6 Hz, 2H), 3.74 (br s, 2H), 3.25-3.19 (m, 4H), 2.59-2.15 (m, 19H), 2.14 (s, 3H), 2.11 (s, 3H), 1.78 (quintet, J=6.9 Hz, 2H). LCMS: m/z 642.54 [M+H]⁺.

Compound 3540, (3-((2,3-dimethyl-1-(3-(4-methylpiperazin-1-yl)propyl)-1H-indol-5-yl)methyl)phenyl)(4-(2-(7-methoxynaphthalen-1-yl)ethyl)piperazin-1-yl)methanone (5%).

¹H NMR (300 MHz, d₆-DMSO): δ 7.76-7.68 (m, 2H), 7.63 (br s, 1H), 7.37-7.31 (m, 3H), 7.29-7.23 (m, 3H), 7.19-7.10 (m, 3H), 6.91 (br d, J=9.6 Hz, 1H), 4.10-3.98 (m, 4H), 3.84 (s, 3H), 3.58 (br s, 4H), 2.88-2.79 (m, 2H), 2.63-2.11 (m, 25H), 1.81-1.68 (m, 2H). LCMS: m/z 672.53 [M+H]⁺.

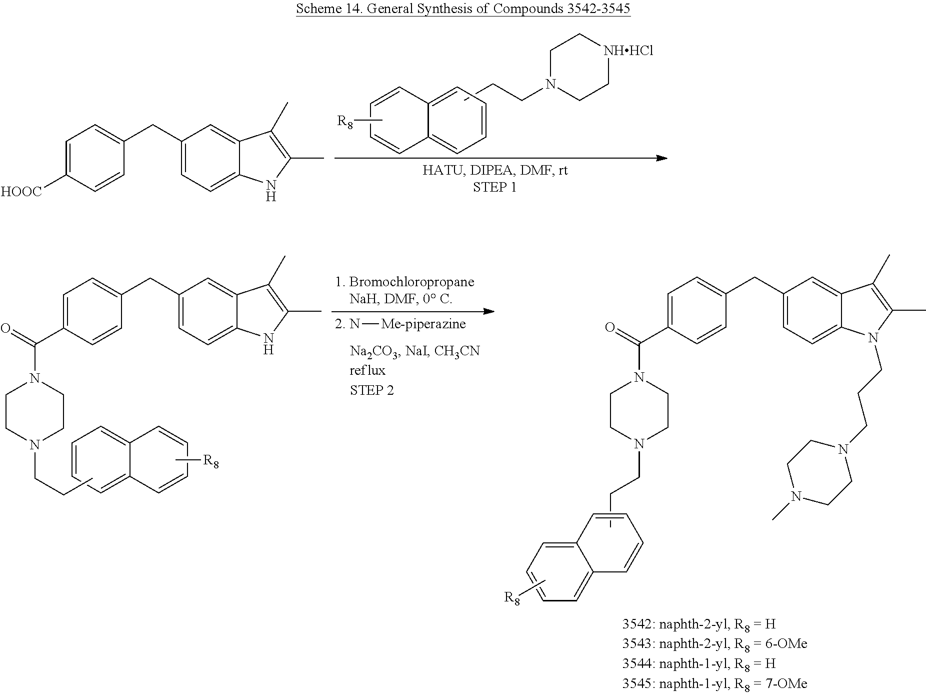

### Step 1: Preparation of (4-((2,3-dimethyl-1H-indol-5-yl)methyl)phenyl)(4-(2-(naphthalen-2-yl)ethyl)piperazin-1-yl) methanone

HATU (680 mg, 1.79 mmol) was added to a stirred solution of 4-((2,3-dimethyl-1H-indol-5-yl)methyl)benzoic acid (250 mg, 0.896 mmol), DIPEA (0.45 mL, 2.68 mmol) in DMF (5 mL) at 0° C. 1-(2-(Naphthalen-2-yl)ethyl)piperazine hydrochloride (276 mg, 0.997 mmol) was added and the reaction mixture was stirred at room temperature for 16 hours. The progress of the reaction was monitored by TLC. After complete consumption of the starting material, the reaction mixture was poured into ice water and extracted using ethyl acetate. The combined organic layers were washed with brine, dried over anhydrous Na₂SO₄ and concentrated under reduced pressure to afford the crude product. The crude compound was purified by flash column chromatography using 5% methanol in CH₂Cl₂ as an eluent to give (4-((2,3-dimethyl-1H-indol-5-yl)methyl)phenyl)(4-(2-(naphthalen-2-yl)ethyl)piperazin-1-yl)methanone as a brown solid (400 mg, 86%). LCMS: m/z 502.24 [M+H]⁺.

Other analogues prepared by this method:
(4-((2,3-dimethyl-1H-indol-5-yl)methyl)phenyl)(4-(2-(6-methoxynaphthalen-2-yl)ethyl)piperazin-1-yl)methanone (92%)
(4-((2,3-dimethyl-1H-indol-5-yl)methyl)phenyl)(4-(2-(naphthalen-1-yl)ethyl)piperazin-1-yl)methanone (90%)
(4-((2,3-dimethyl-1H-indol-5-yl)methyl)phenyl)(4-(2-(7-methoxynaphthalen-1-yl)ethyl)piperazin-1-yl)methanone (90%)

### Step 2-1: Preparation of (4-((1-(3-chloropropyl)-2,3-dimethyl-1H-indol-5-yl)methyl)phenyl) (4-(2-(naphthalen-2-yl)ethyl)piperazin-1-yl)methanone

NaH (15 mg, 0.63 mmol) was added portion-wise to a stirred solution of (4-((2,3-dimethyl-1H-indol-5-yl)methyl)phenyl)(4-(2-(naphthalen-2-yl)ethyl)piperazin-1-yl)methanone (150 mg, 0.290 mmol) in DMF (6 mL) at 0° C. The mixture was allowed to warm to room temperature for 30 minutes. To this, bromochloropropane (0.15 mL, 1.5 mmol) was added dropwise at 0° C. The reaction mixture was stirred at room temperature for 3 hours. After complete consumption of the starting material, ice-cold water was added into the reaction mixture, which was then extracted with ethyl acetate. The organic layer was washed with brine solution, dried over anhydrous Na₂SO₄ and concentrated under reduced pressure to afford the crude product. The crude compound was purified by flash column chromatography using ethyl acetate as an eluent to afford (4-((1-(3-chloropropyl)-2,3-dimethyl-1H-indol-5-yl)methyl)phenyl)(4-(2-(naphthalen-2-yl)ethyl)piperazin-1-yl)methanone as a yellow liquid (170 mg, 98%). LCMS: m/z 578.43 [M+H]⁺.

Other analogues prepared by this method:
(4-((1-(3-chloropropyl)-2,3-dimethyl-1H-indol-5-yl)methyl)phenyl)(4-(2-(6-methoxynaphthalen-2-yl)ethyl)piperazin-1-yl)methanone (42%)
(4-((1-(3-chloropropyl)-2,3-dimethyl-1H-indol-5-yl)methyl)phenyl)(4-(2-(naphthalen-1-yl)ethyl)piperazin-1-yl)methanone (36%)
(4-((1-(3-chloropropyl)-2,3-dimethyl-1H-indol-5-yl)methyl)phenyl)(4-(2-(7-methoxynaphthalen-1-yl)ethyl)piperazin-1-yl)methanone (44%)

### Step 6-2: Preparation of Compound 3542, (4-((2,3-dimethyl-1-(3-(4-methylpiperazin-1-yl)propyl)-1H-indol-5-yl)methyl)phenyl) (4-(2-(naphthalen-2-yl)ethyl)piperazin-1-yl)methanone

To a stirred solution of (4-((1-(3-chloropropyl)-2,3-dimethyl-1H-indol-5-yl)methyl)phenyl)(4-(2-(naphthalen-2-yl)ethyl)piperazin-1-yl)methanone (170 mg, 0.284 mmol) in acetonitrile (5 mL) were added sodium iodide (88.09 mg, 0.58 mmol) and sodium carbonate (155 mg, 1.47 mmol), followed by N-methylpiperazine (117 mg, 1.17 mmol), at room temperature. The reaction mixture was heated to 75° C. for 16 hours. After complete consumption of the starting material, the reaction mixture was cooled to room temperature, diluted with ethyl acetate (40 mL) and washed with water, followed by brine solution. The organic layer was dried over anhydrous Na₂SO₄ and concentrated under reduced pressure to afford the crude product. The crude compound was purified by flash column chromatography using 5% Methanol-DCM as an eluent to afford (4-((2,3-dimethyl-1-(3-(4-methylpiperazin-1-yl)propyl)-1H-indol-5-yl)methyl)phenyl)(4-(2-(naphthalen-2-yl)ethyl)piperazin-1-yl)methanone (Compound 3524) as a yellow sticky liquid (25 mg, 14%).

¹H NMR (400 MHz, CD₃OD): δ 8.07 (br d, J=8.8 Hz, 1H), 7.85 (br d, J=7.6 Hz, 1H), 7.72 (dd, J=7.2 Hz, 2.0 Hz, 1H), 7.53-7.44 (m, 2H), 7.41-7.31 (m, 6H), 7.24 (br s, 1H), 7.21 (d, J=8.4 Hz, 1H), 6.92 (dd, J=8.4 Hz, 1.6 Hz, 1H), 4.13 (t, J=6.8 Hz, 2H), 4.07 (s, 2H), 3.82 (br s, 2H), 3.59 (br s, 2H), 3.32-3.25 (m, 2H), 2.77-2.35 (m, 14H), 2.34 (s, 3H), 2.31 (t, J=6.8 Hz, 2H), 2.26 (s, 3H), 2.18 (s, 3H), 1.89 (quintet, J=6.8 Hz, 2H).

LCMS: m/z 642.57 [M+H]⁺.

Other analogues prepared by this method:

Compound 3543, (4-((2,3-dimethyl-1-(3-(4-methylpiperazin-1-yl)propyl)-1H-indol-5-yl)methyl)phenyl)(4-(2-(6-methoxynaphthalen-2-yl)ethyl)piperazin-1-yl)methanone (12%).

¹H NMR (300 MHz, d₆-DMSO): δ 7.75-7.70 (m, 2H), 7.63 (br s, 1H), 7.34 (br d, J=9.9 Hz, 1H), 7.31-7.23 (m, 7H), 7.12 (dd, J=8.7 Hz, 2.1 Hz, 1H), 6.92 (brd, J=9.6 Hz, 1H), 4.07 (t, J=6.6 Hz, 2H), 4.02 (br s, 2H), 3.85 (s, 3H), 3.50 (br s, 4H), 2.89-2.81 (m, 2H), 2.64-2.58 (m, 2H), 2.51-2.13 (m, 23H), 1.75 (quintet, J=6.6 Hz, 2H). LCMS: m/z 672.0 [M+H]⁺.

Compound 3544, (4-((2,3-dimethyl-1-(3-(4-methylpiperazin-1-yl)propyl)-1H-indol-5-yl)methyl)phenyl)(4-(2-(naphthalen-1-yl)ethyl)piperazin-1-yl)methanone (33%).

¹H NMR (300 MHz, d₆-DMSO): δ 8.04 (br d, J=8.4 Hz, 1H), 7.91 (dd, J=7.5 Hz, 1.5 Hz, 1H), 7.77 (br d, 6.9 Hz, 1H), 7.58-7.45 (m, 2H), 7.44-7.37 (m, 2H), 7.31-7.23 (m, 6H), 6.92 (br d, J=9.6 Hz, 1H), 4.07 (t, J=7.2 Hz, 2H), 4.02 (br s, 2H), 3.55 (br s, 4H), 3.24-3.18 (m, 2H), 2.67-2.60 (m, 2H), 2.47-2.12 (m, 23H), 1.75 (quintet, J=6.9 Hz, 2H). LCMS: m/z 642.1 [M+H]⁺.

Compound 3545, (4-((2,3-dimethyl-1-(3-(4-methylpiperazin-1-yl)propyl)-1H-indol-5-yl)methyl)phenyl)(4-(2-(7-methoxynaphthalen-1-yl)ethyl)piperazin-1-yl)methanone (33%).

¹H NMR (300 MHz, d₆-DMSO): δ 7.84 (d, J=9.2 Hz, 1H), 7.70 (br d, J=8.4 Hz, 1H), 7.36-7.23 (m, 7H), 7.20-7.15 (m, 3H), 6.90 (dd, J=8.4 Hz, 1.6 Hz, 1H), 4.06-3.99 (m, 4H), 3.61 (br s, 4H), 3.18-3.11 (m, 2H), 2.63-2.11 (m, 25H), 1.71 (quintet, J=7.2 Hz, 2H). LCMS: m/z 672.1 [M+H]⁺.

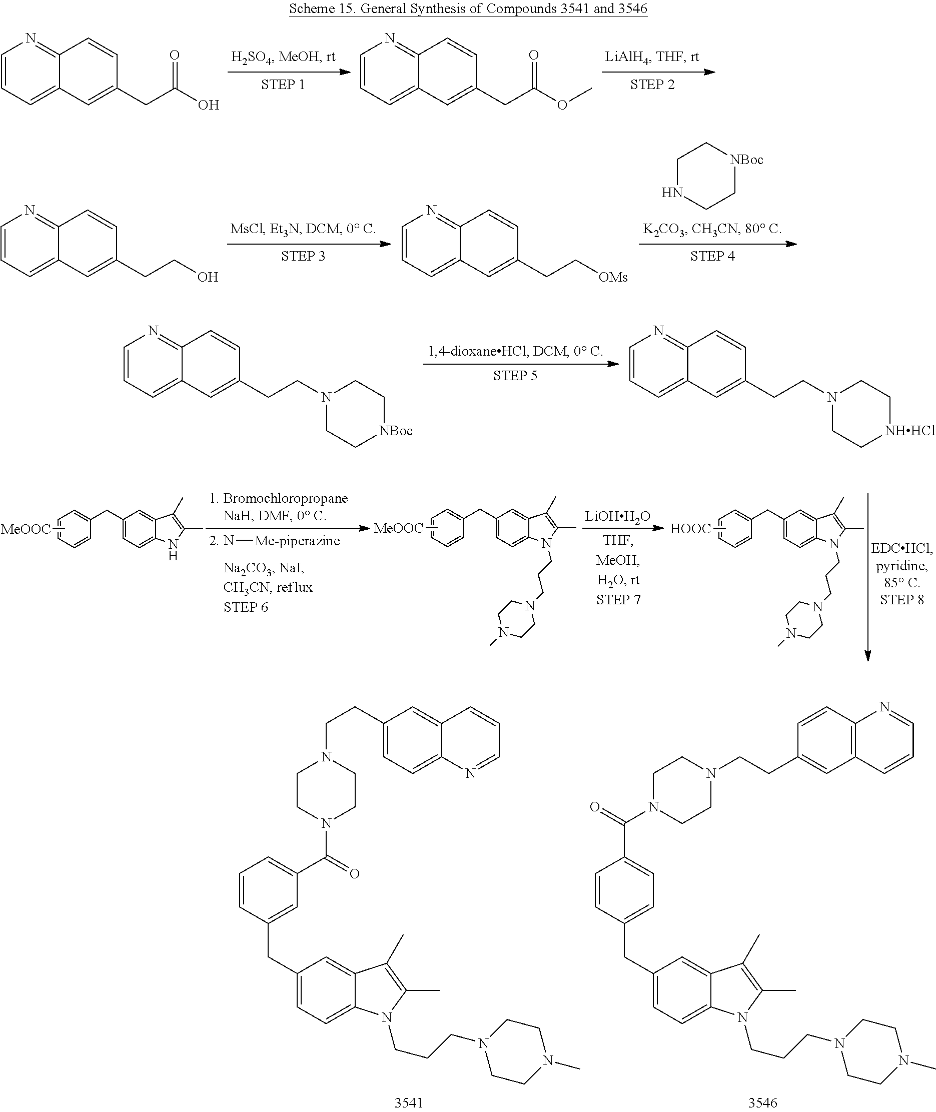

### Step 1: Preparation of methyl 2-(quinolin-6-yl)acetate

To a solution of 2-(quinolin-6-yl)acetic acid (2.0 g, 11 mmol) in methanol (40 mL) was added concentrated sulphuric acid (0.2 mL). The mixture was stirred at room temperature for 6 hours. The reaction mass was poured into water and the resulting solution was neutralised with saturated NaHCO₃ solution and extracted with ethyl acetate. The organic layer was washed with water and brine, dried over Na₂SO₄ and evaporated in vacuo to give methyl 2-(quinolin-6-yl)acetate (1.8 g, 85%). LCMS: m/z 202.25 [M+H]⁺.

### Step 2: Preparation of 2-(quinolin-6-yl)ethan-1-ol

To a mixture of LiAlH₄ (680 mg, 17.9 mmol) in THF (50 mL) at 0° C. was added methyl 2-(quinolin-6-yl)acetate (1.8 g, 8.9 mmol). The mixture was stirred for 10 minutes at this temperature, after which the reaction mass was slowly warmed to room temperature and stirred for 4 hours. After complete consumption of the starting material as determined by TLC, the reaction mixture was quenched with ethyl acetate (3 mL) and saturated ammonium chloride solution (20 mL) at 0° C., filtered and concentrated to give 2-(quinolin-6-yl)ethan-1-ol (1.3 g, 84%). LCMS: m/z 174.28 [M+H]⁺.

### Step 3: Preparation of 2-(quinolin-6-yl)ethyl methanesulphonate

To a solution of 2-(quinolin-6-yl)ethan-1-ol (1.3 g, 7.5 mmol) in dichloromethane (25 mL) at 0° C., was added triethylamine (5.2 mL, 38 mmol) and methanesulfonyl chloride (1.2 mL, 15 mmol). The mixture was stirred at this temperature for 3 hours. After complete consumption of the starting material as determined by TLC, the reaction mixture was poured into water and extracted with ethyl acetate. The organic extracts were washed with water and brine, dried over Na₂SO₄ and evaporated to give 2-(quinolin-6-yl)ethyl methanesulphonate (1.7 g, crude) which was used without further purification.

### Step 4: Preparation of tert-butyl 4-(2-(quinolin-6-yl)ethyl)piperazine-1-carboxylate

To a solution of N-Boc piperazine (1.3 g, 7.2 mmol) in DMF (60 mL), were added K₂CO₃ (1.49 g, 10.8 mmol) and 2-(quinolin-6-yl)ethyl methanesulphonate (0.9 g, 3.6 mmol) at room temperature. The reaction mixture was heated to 80° C. for 12 hours. After complete consumption of the starting material as determined by TLC, ice-cold water was added into the reaction mixture, which was then extracted with ethyl acetate. The combined organic layers were washed with water and brine, dried over anhydrous Na₂SO₄ and concentrated under reduced pressure to afford the crude product. The crude compound was purified by flash column chromatography using 40-50% ethyl acetate in petroleum ether as an eluent to give tert-butyl 4-(2-(quinolin-6-yl)ethyl)piperazine-1-carboxylate (0.8 g, 66%). LCMS: m/z 342.05 [M+H]⁺.

### Step 5: Preparation of 6-(2-(piperazin-1-yl)ethyl)quinoline hydrochloride

To a stirred solution of tert-butyl 4-(2-(quinolin-6-yl)ethyl)piperazine-1-carboxylate (800 mg, 2.3 mmol) in DCM (50 mL) was added 1,4-dioxane/HCl (25 mL, ˜4 M) at 0° C. The reaction mixture was stirred for 10 minutes, then the temperature was raised to room temperature, which was maintained for 12 hours. After complete consumption of the starting material as determined by TLC, the reaction mass was concentrated, diethyl ether was added and the mixture was filtered to afford 6-(2-(piperazin-1-yl)ethyl)quinoline hydrochloride (0.6 g, crude). LCMS: m/z 242.2 [(M−HCl)+H]⁺.

### Step 6-1: Preparation of methyl 4-((1-(3-chloropropyl)-2,3-dimethyl-1H-indol-5-yl)methyl)benzoate

NaH (400 mg, 16.7 mmol) was added portionwise to a stirred solution of methyl 4-((2,3-dimethyl-1H-indol-5-yl)methyl)benzoate (830 mg, 2.8 mmol) in DMF (10 mL) at 0° C. The mixture was allowed to warm to room temperature for 30 minutes. To this, bromochloropropane (0.6 g, 3.8 mmol) was added dropwise at 0° C. and the reaction mixture was allowed to stir at this temperature for 2 hours. After complete consumption of the starting material, ice-cold water was added into the reaction mixture, which was then extracted with ethyl acetate. The organic layer was washed with brine solution, dried over anhydrous Na₂SO₄ and concentrated under reduced pressure to afford the crude product. The crude compound was purified by flash column chromatography to afford methyl 4-((1-(3-chloropropyl)-2,3-dimethyl-1H-indol-5-yl)methyl)benzoate (380 mg, 36%).

¹H NMR (400 MHz, CDCl₃): δ 7.93 (d, J=8.4 Hz, 2H), 7.30-7.27 (m, 3H), 7.20 (d, J=8.0 Hz, 1H), 6.94 (dd, J=8.0 Hz, 1.6 Hz, 1H), 4.22 (t, J=6.8 Hz, 2H), 4.18 (s, 2H), 3.89 (s, 2H), 3.50 (t, J=6.0 Hz, 2H), 2.35 (s, 3H), 2.22-2.16 (m, 5H). LCMS: m/z 370.1 [M+H]⁺.

Other analogues prepared by this method:
methyl 3-((1-(3-chloropropyl)-2,3-dimethyl-1H-indol-5-yl)methyl)benzoate (32%).

### Step 6-2: Preparation of methyl 4-((2,3-dimethyl-1-(3-(4-methylpiperazin-1-yl)propyl)-1H-indol-5-yl)methyl)benzoate

To a stirred solution of methyl 4-((1-(3-chloropropyl)-2,3-dimethyl-1H-indol-5-yl)methyl)benzoate (380 mg, 1.0 mmol) in acetonitrile (12 mL) at room temperature, sodium iodide (380 mg, 2.5 mmol) and sodium carbonate (270 mg, 2.5 mmol) were added, followed by N-methylpiperazine (250 mg, 2.5 mmol). The reaction mixture was heated to 80° C. for 12 hours. After complete consumption of the starting material, the reaction mixture was cooled to room temperature, diluted with ethyl acetate, washed with water and brine solution, dried over anhydrous Na₂SO₄ and concentrated under reduced pressure to afford the crude product. The crude compound was purified by flash column chromatography to afford methyl 4-((2,3-dimethyl-1-(3-(4-methylpiperazin-1-yl)propyl)-1H-indol-5-yl)methyl)benzoate (310 mg, 67%).

¹H NMR (400 MHz, CDCl₃): δ 7.93 (d, J=8.4 Hz, 2H), 7.29-7.26 (m, 3H), 7.18 (d, J=8.4 Hz, 1H), 6.93 (dd, J=8.4 Hz, 1.2 Hz, 1H), 4.12-4.07 (m, 4H), 3.89 (s, 3H), 2.82-2.31 (m, 16H), 2.20 (s, 3H), 1.92-1.85 (m, 2H). LCMS: m/z 434.3 [M+H]⁺.

Other analogues prepared by this method:
methyl 3-((2,3-dimethyl-1-(3-(4-methylpiperazin-1-yl)propyl)-1H-indol-5-yl)methyl)benzoate (67%).

### Step 7: Preparation of 4-((2, 3-dimethyl-1-(3-(4-methylpiperazin-1-yl)propyl)-1H-indol-5-yl)methyl)benzoic acid

To a solution of methyl 4-((2,3-dimethyl-1-(3-(4-methylpiperazin-1-yl)propyl)-1H-indol-5-yl)methyl)benzoate (200 mg, 0.46 mmol) in THF:H₂O:MeOH (4:1:1, 5 mL) was added LiOH.H₂O (38 mg, 0.91 mmol) at room temperature. The reaction mixture was stirred for 12 hours. After complete consumption of the starting material, the reaction mass was concentrated and then partitioned between ethyl acetate and water. The aqueous layer was collected and acidified with saturated citric acid solution at 0° C. The precipitate thus obtained was collected by filtration and dried over vacuum to afford 4-((2,3-dimethyl-1-(3-(4-methylpiperazin-1-yl)propyl)-1H-indol-5-yl)methyl)benzoic acid (160 mg, 84%).

Other analogues prepared by this method:
3-((2,3-dimethyl-1-(3-(4-methylpiperazin-1-yl)propyl)-1H-indol-5-yl)methyl)benzoic acid (75%). LCMS: m/z 420.3 [M+H]⁺

### Step 8: Preparation of Compound 3541, (3-((2,3-dimethyl-1-(3-(4-methylpiperazin-1-yl)propyl)-1H-indol-5-yl)methyl)phenyl) (4-(2-(quinolin-6-yl)ethyl)piperazin-1-yl)methanone

To a solution of 3-((2,3-dimethyl-1-(3-(4-methylpiperazin-1-yl)propyl)-1H-indol-5-yl)methyl)benzoic acid (90 mg, 0.21 mmol) and 6-(2-(piperazin-1-yl)ethyl)quinoline hydrochloride (175 mg, 0.63 mmol) in pyridine (3 mL), was added EDC.HCl (230 mg, 1.2 mmol). The mixture was stirred at 80° C. for one hour. The reaction mixture was then partitioned between ethyl acetate and water. The organic layer was washed with water and brine, then dried, concentrated and purified by preparative HPLC to afford (3-((2,3-dimethyl-1-(3-(4-methylpiperazin-1-yl)propyl)-1H-indol-5-yl)methyl)phenyl)(4-(2-(quinolin-6-yl)ethyl)piperazin-1-yl)methanone (Compound 3541) (8 mg, 6%).

¹H NMR (400 MHz, DMSO-d₆): δ 8.83 (m, 1H), 8.28 (br d, J=7.6 Hz, 1H), 7.94 (d, J=8.4 Hz, 1H), 7.79 (br s, 1H), 7.68-7.63 (m, 1H), 7.55-7.49 (m, 1H), 7.38-7.32 (m, 2H), 7.28-7.22 (m, 2H), 7.19-7.13 (m, 2H), 6.91 (br d, J=8.8 Hz, 1H), 4.09-4.00 (m, 4H), 3.41-3.19 (m, 4H), 2.97-2.89 (m, 2H), 2.64-2.59 (m, 2H), 2.39-2.08 (m, 23H), 1.77-1.69 (m, 2H). LCMS: m/z 643.4 [M+H]⁺.

Other analogues prepared by this method:

Compound 3546, (4-((2,3-dimethyl-1-(3-(4-methylpiperazin-1-yl)propyl)-1H-indol-5-yl)methyl)phenyl)(4-(2-(quinolin-6-yl)ethyl)piperazin-1-yl)methanone (2%).

¹H NMR (400 MHz, DMSO-d₆): δ 8.83 (dd, J=4.4 Hz, 2.0 Hz, 1H), 8.27 (br d, J=7.6 Hz, 1H), 7.93 (d, J=8.4 Hz, 1H), 7.78 (br s, 1H), 7.65 (dd, J=8.8 Hz, 2.0 Hz, 1H), 7.49 (dd, J=8.4 Hz, 4.0 Hz, 1H), 7.31-7.24 (m, 6H), 6.91 (brd, J=9.2 Hz, 1H), 4.11-4.02 (m, 4H), 3.58 (br s, 4H), 2.97-2.91 (m, 2H), 2.68-2.61 (m, 2H), 2.50-2.11 (m, 20H), 1.85 (s, 3H), 1.79-1.72 (m, 2H). LCMS: m/z 643.4 [M+H]⁺.

### Activity of Anti-Tropomyosin Compounds as Monotherapy

### Anti-Proliferative Activity of Compounds of the Invention

In silico modelling has identified binding sites on tropomyosin Tpm3.1, yielding the series of tropomyosin inhibitors the subject of the present invention. Inhibition of Tpm3.1 in tumour cells results in disruption of the actin cytoskeleton and ultimately cell death.

The ability of compounds 3501-3540 and 3542-3545 to inhibit the proliferation of cancer cells representative of neuroblastoma, melanoma, prostate cancer, colorectal cancer, non-small cell lung carcinoma, and triple negative breast cancer was assessed. Briefly, a pre-determined number of cells as calculated from cell growth assays for each of the cell lines employed were seeded into their respective culture medium (using ATCC culture parameters—http://www.atcc.org) and cultured for 24 hours at 37° C. and 5% CO₂ in 96-well culture plates. Once attached, each cell line was then exposed to increasing concentrations of each respective analogue (0.03, 0.3, 3 and 30 μM for compounds in Table 1; 0.1, 0.3, 1, 3, 10 and 30 μM for compounds in Table 2), cultured for a further 72 hours and exposed to cell-titre luminescent reagent (100 μL/well) for a further 30 min) to assess for cell viability. Luminescence was captured using an EnVision multilabel reader and the data for each analogue concentration was normalized, as a percentage, to the no treatment control. For compounds 3501, 3503, 3505-08, 3512-18, 3520, 3522-24 and 3531-36, semi-log plots of Percent of Control versus concentration were prepared and IC₅₀ determined using linear regression analysis. For compounds 3502, 3504, 3509-11, 3519, 3521, 3525-30, 3537-40 and 3542-45, cell viability was normalized to control (vehicle alone) and dose-response curves, and half maximal effective concentration (EC₅₀) values were determined using Graph Pad Prism 6 (nonlinear regression sigmoidal dose-response variable slope).

TABLE 1
Anti-proliferative activity of compounds of the invention against a range of
somatic cancer cells.

| | IC₅₀/μM | | | | | | |
|---|---|---|---|---|---|---|---|
| Compound | Neuroblastoma | Melanoma | Prostate | | Colorectal | Lung (NSLC) | Breast |
| ID | SK-N-SH | SK-Mel-28 | DU145 | PC3 | CaCo2 | A549 | MDA-MB-231 |
| 3501 | 3.1 | 1.3 | 2.6 | 2.4 | 3.1 | 3.7 | 2.1 |
| 3503 | 2.1 | 1.0 | 2.1 | 2.4 | 3.4 | 1.4 | 1.0 |
| 3505 | 2.6 | 2.5 | 3.4 | 2.6 | 3.6 | 3.1 | 2.8 |
| 3506 | 3.7 | 1.3 | 3.2 | 2.1 | 2.7 | 3.0 | 1.9 |
| 3507 | 1.4 | 1.0 | 1.3 | 1.2 | 2.5 | 1.2 | 1.4 |
| 3508 | 1.7 | 1.6 | 3.1 | 2.4 | 3.1 | 1.8 | 1.8 |
| 3512 | 1.8 | 0.7 | 1.3 | 2.1 | 3.2 | 1.3 | 1.2 |
| 3513 | 3.3 | 3.4 | 3.8 | 4.0 | 3.1 | 3.3 | 3.6 |
| 3514 | 1.3 | 1.3 | 3.8 | 3.5 | 3.1 | 1.4 | 1.3 |
| 3515 | 3.4 | 2.7 | 4.0 | 4.9 | 4.6 | 4.3 | 3.3 |
| 3516 | 1.4 | 2.6 | 3.0 | 3.5 | 2.1 | 2.2 | 1.3 |
| 3517 | 4.5 | 3.7 | 7.1 | 21.1 | >30 | 4.4 | 5.3 |
| 3518 | 2.3 | 2.4 | 3.2 | 4.1 | 2.5 | 3.0 | 2.1 |
| 3520 | 2.3 | 2.5 | 2.7 | 2.3 | 3.5 | 2.4 | 2.8 |
| 3522 | 2.0 | 1.0 | 2.4 | 2.5 | 3.0 | 1.6 | 1.3 |
| 3523 | 2.1 | 0.8 | 2.1 | 2.4 | 3.1 | 1.4 | 1.1 |
| 3524 | 1.4 | 1.1 | 1.8 | 0.9 | 1.7 | 0.9 | 1.1 |
| 3531 | 3.4 | 2.8 | 3.9 | 4.6 | 4.2 | 3.9 | 3.5 |
| 3532 | 2.3 | 2.7 | 4.0 | 4.2 | 3.1 | 2.2 | 1.9 |
| 3533 | 3.3 | 4.8 | 3.7 | 3.3 | 3.9 | 3.7 | 4.2 |
| 3534 | 1.4 | 1.2 | 1.4 | 2.8 | 3.2 | 1.5 | 1.6 |
| 3535 | 2.8 | 3.3 | 3.8 | 3.7 | 3.0 | 2.7 | 2.6 |
| 3536 | 2.9 | 2.7 | 3.6 | 4.3 | 12.9 | 3.1 | 2.5 |

TABLE 2
Anti-proliferative activity of compounds of the invention against a range of
somatic cancer cells.

| | IC₅₀/μM | | | | | | |
|---|---|---|---|---|---|---|---|
| Compound | Neuroblastoma | Melanoma | Prostate | | Colorectal | Lung (NSLC) | Breast |
| ID | SK-N-SH | SK-Mel-28 | DU145 | PC3 | CaCo2 | A549 | MDA-MB-231 |
| 3502 | 4.6 | 2.6 | 4.9 | 5.4 | 5.8 | 5.0 | 4.9 |
| 3504 | 1.5 | 1.1 | 1.4 | 2.0 | 1.8 | 1.6 | 1.5 |
| 3509 | 5.3 | 5.1 | 5.9 | 6.9 | 5.2 | 4.7 | 5.0 |
| 3510 | 4.6 | 4.9 | 8.0 | 8.0 | 5.8 | 6.0 | 20.4 |
| 3511 | 2.1 | 6.5 | 3.3 | 2.6 | 3.5 | 2.6 | 19.3 |
| 3519 | 2.6 | 2.5 | 4.3 | 4.9 | 5.6 | 4.4 | 5.0 |
| 3521 | 4.2 | 4.1 | 5.7 | 6.3 | 6.0 | 5.9 | 6.6 |
| 3525 | 1.9 | 1.7 | 4.1 | 4.5 | 4.1 | 3.8 | 4.3 |
| 3526 | 4.8 | 4.8 | 9.0 | 15.6 | 5.3 | 3.0 | 13.4 |
| 3527 | 3.2 | 4.1 | 4.5 | 7.2 | 4.1 | 5.0 | 4.8 |
| 3528 | 4.3 | 3.8 | 5.2 | 7.3 | 4.6 | 6.6 | 5.9 |
| 3529 | 2.0 | 2.4 | 4.2 | 4.8 | 4.2 | 4.4 | 4.5 |
| 3530 | 1.6 | 3.9 | 4.5 | 4.8 | 4.3 | 2.4 | 2.1 |
| 3537 | 4.2 | 4.8 | 5.8 | 8.0 | 5.0 | 3.6 | 4.8 |
| 3538 | 4.8 | 3.3 | 4.2 | 3.9 | 5.7 | 4.3 | 3.6 |
| 3539 | 1.7 | 2.2 | 3.9 | 3.0 | 4.6 | 3.9 | 2.8 |
| 3540 | 1.7 | 1.9 | 4.6 | 2.3 | 4.0 | 5.0 | 2.3 |
| 3542 | 1.6 | 1.4 | 3.9 | 3.6 | 3.9 | 3.1 | 2.8 |
| 3543 | 1.6 | 2.0 | 1.7 | 1.8 | 1.5 | 1.5 | 1.3 |
| 3544 | 7.0 | 7.2 | 9.0 | 8.1 | 13.5 | 5.7 | 8.2 |
| 3545 | 2.1 | 3.2 | 3.6 | 3.0 | 3.1 | 4.2 | 2.3 |

The anti-proliferative activity of compound 3507 was further evaluated in cell lines representative of melanoma, prostate cancer, leukaemia and neuroblastoma. Cell viability after 72 hours exposure to increasing concentrations of compound 3507 was measured using an MTS viability assay. Cell viability was normalized to control (vehicle alone) and dose-response curves, and relative inhibitory concentration (IC₅₀) values were determined using GraphPad Prism 6.

TABLE 3
Anti-proliferative activity of compound 3507
against a range of somatic cancer cells.

| Cancer Type | Cell Line | IC₅₀/μM |
|---|---|---|
| Melanoma | A2058 | 4.06 |
| | BL | 5.83 |
| | D20 | 5.01 |
| | MM329 | 3.80 |
| | MM415 | 4.67 |
| | MM96L | 3.27 |
| | SKMEL13 | 3.72 |
| Prostate | LNCaP | 0.59 |
| | BPH-1 | 0.72 |
| | P4E6 | 0.87 |
| Leukemia | THP-1 | 0.80 |
| | HL-60 | 1.16 |
| | K562 | 0.95 |
| Neuroblastoma | CHLA-20 | 6.90 |
| | CHP-134 | 7.20 |
| | CHLA-90 | 4.24 |
| | SK-N-Be(2) | 5.47 |

### Impact of Compounds of the Invention on the Actin Cytoskeleton

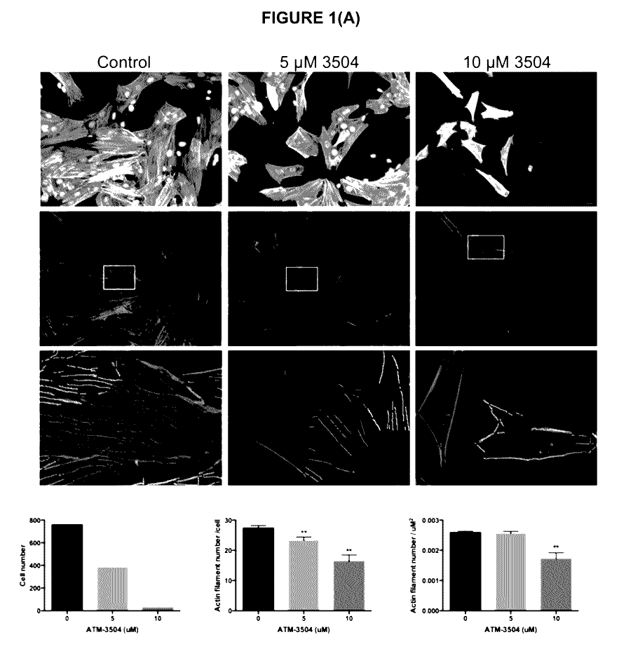

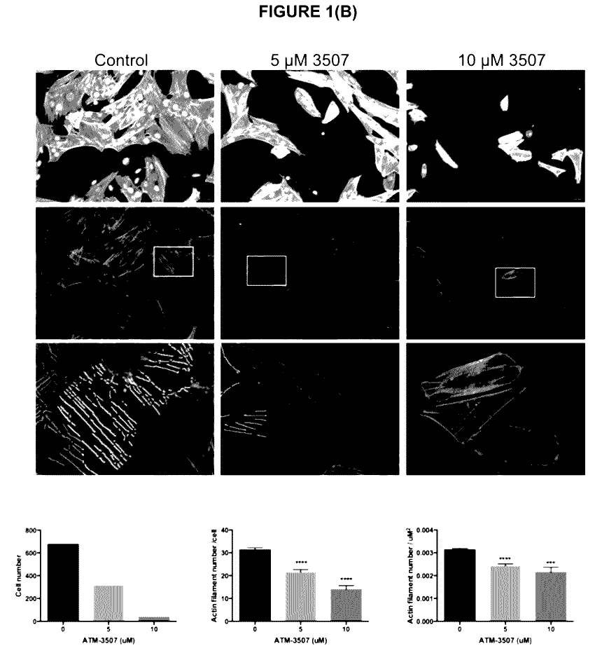

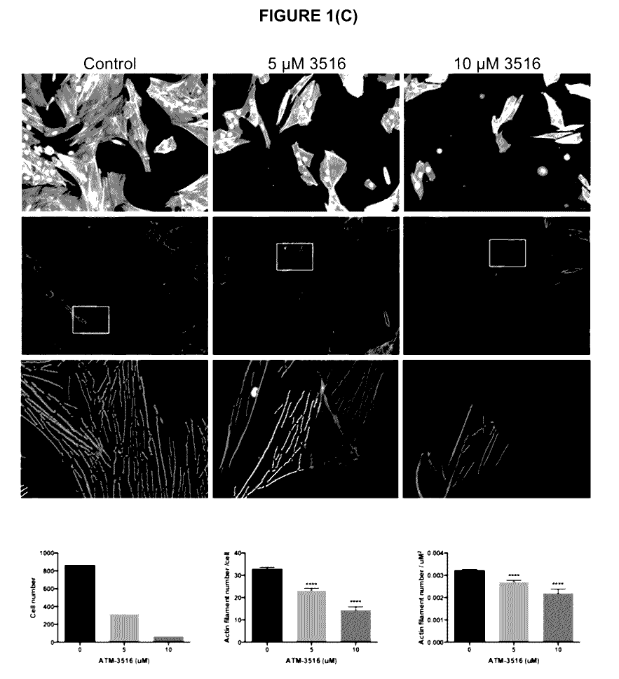

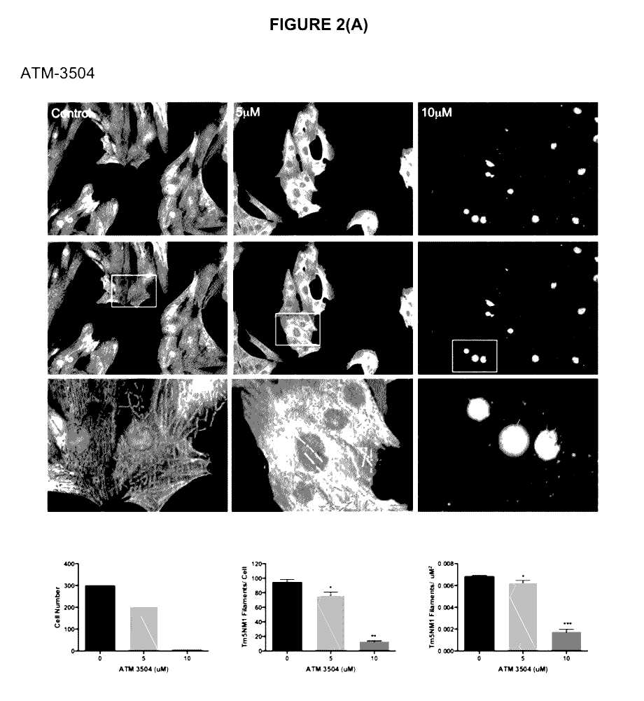

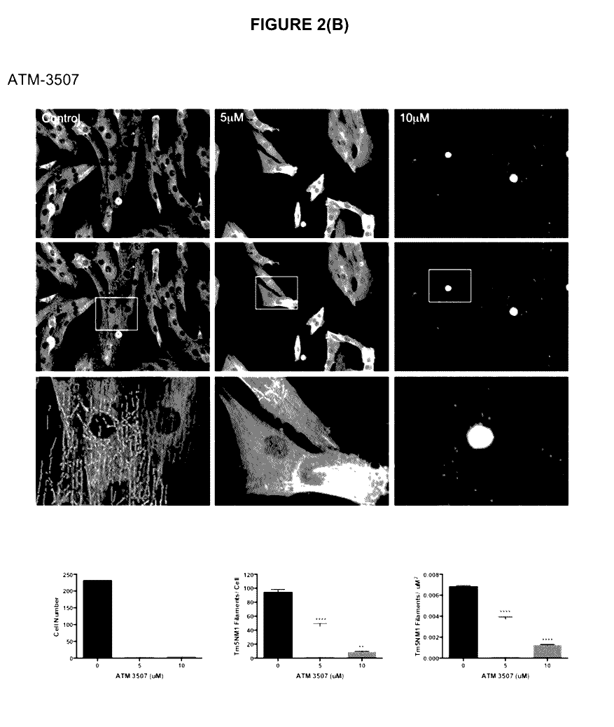

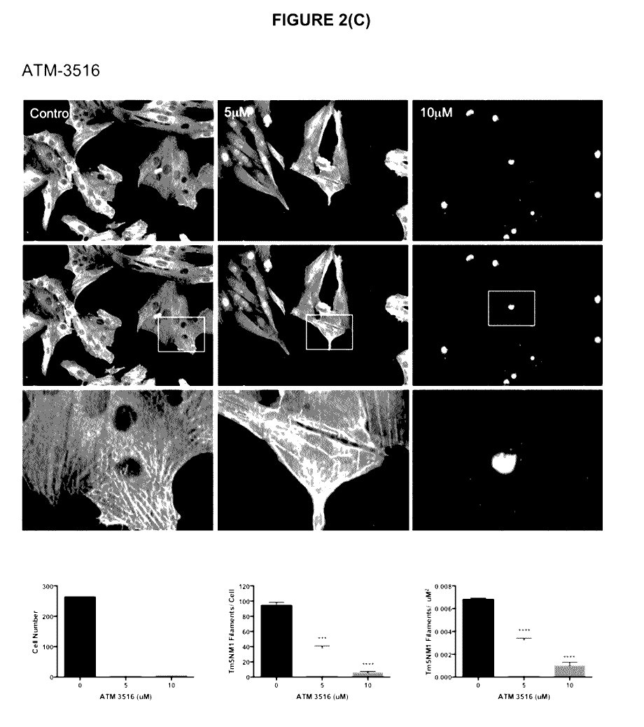

The ability of compounds 3504, 3507 and 3516 to disrupt the total actin cytoskeleton (FIG. 1) and to specifically target Tpm3.1-containing actin filaments (FIG. 2) was assessed in vitro using the microfilament disruption assay.

Briefly, SK-N-SH neuroblastoma cells were seeded at 1800 cells/well in a 384 Perkin Elmer High Content Imaging “View” plate and left to plate down 24 hours prior to treatment. Cells were then treated with 0-40 μM of the test compounds (1:2 serial dilution in a 10 point dose response). 24 hours post treatment, cells were fixed with 4% w/v paraformaldehyde (PBS), permeabilized with Triton-X-100 and stained with 488-Atto-Phallodin and DAPI to visualize the actin filament bundles and the nucleus, or with γ9d (sheep polycolonal, 1:100) followed by 488-conjugated secondary (1:1000) and DAPI to visualize the Tpm3.1 containing filament bundles and the nucleus, respectively. Single plane images were obtained on the Perkin Elmer Opera confocal microscope using a 20× objective. Twelve fields of view per condition were imaged. Images were then exported and changes in the organization and numbers of actin filaments within the cell were quantitated using a linear feature detection algorithm developed by the CSIRO (Vindin et al., 2014). This algorithm detects the “ridge lines” or “peaks” in local pixel intensity in the cell image. It is these “ridge lines” that correspond to actin filament bundles and allow us to quantitate the number of filaments per cell.

Data demonstrate that compounds 3504, 3507 and 3516 disrupt both the total actin cytoskeleton and Tpm3.1-containing actin filaments in a dose-dependent manner.

In order to demonstrate that the compounds of the invention impaired Tpm3.1 function, the impact of compound 3507 on Tpm3.1-regulated actin filament depolymerisation was assessed using a well-characterized pyrene-based actin filament depolymerisation assay (Broschat, 1990; Kostyukova and Hitchcock, 2004). A brief overview and rationale of the assay is as follows; to promote depolymerisation, pyrene-labelled actin filaments were diluted below the critical concentration of the pointed end (0.5 μM, as defined by Pollard et al., 1986). A decline in fluorescence was measured over time as actin monomers dissociate. It is well established that in the presence of Tpm3.1 the rate of actin depolymerisation is significantly reduced (Bonello 2013). Therefore, any compound, which interacts with, and impacts Tpm3.1 function, would nullify the protective effect of Tpm3.1 on actin depolymerisation.

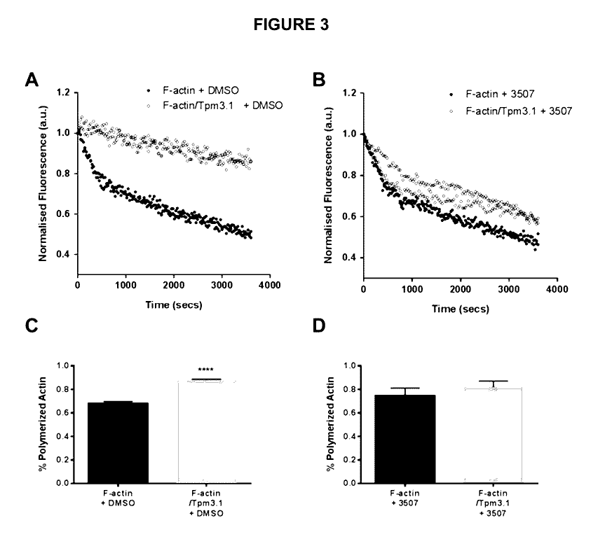

For all assays the depolymerisation of F-actin alone and F-actin-coated with the human homologue of Tpm3.1 was used as a comparative control. Briefly, Tpm3.1 was pre-incubated with F-actin for 20 minutes prior to diluting the filaments, to allow for proper assembly of the Tpm3.1 polymer. As expected, in the presence of saturating amounts of Tpm3.1, the initial rate (V₀) of F-actin depolymerisation was significantly slower for Tpm3.1-containing actin filaments (FIGS. 3A and C, p<0.0001).

The depolymerisation of F-actin alone and F-actin-coated with Tpm3.1 was then measured in the presence of test compound and initial rates of depolymerisation were compared. Tpm3.1 was pre-incubated with 50 μM 3507 prior to being added to the actin filaments as previously described. In the presence of compound 3507 the ability of Tpm3.1 to polymerize and protect actin was impaired and the rate of depolymerisation was not significantly different to F-actin (FIGS. 3B and D). These data demonstrate that compound 3507 interacts with and impairs Tpm3.1 function.

### Impact of Compounds of the Invention on Release of Cytokines

The ability of compounds 3507, 3520, 3534 and 3538 to inhibit the release of cytokines TNF-α, IFN-γ, IL-6, IL-21, IL-17A and IL-23 was evaluated in vitro (Tables 4 and 5). Briefly, human peripheral blood mononuclear cells (PBMCs) were isolated from human peripheral blood by Histopaque density gradient centrifugation. The freshly isolated PBMCs were seeded at 50,000 cells/well in a 96-well half area plate. PBMCs were dosed with the test compounds (at 10 μM, 1 μM and 0.1 μM) and then incubated at 37° C. and 5% CO₂ for 2 hours. To stimulate release of the cytokines IFN-γ, IL-21, IL-17A and IL-23, the PBMCs were treated with 50 ng/mL of phorbol 12-myristate 13-acetate (PMA) and 1 μg/mL of ionomycin and to stimulate the release of TNF-α and IL-6, PBMCs were treated with 100 ng/mL of lipopolysaccharide (LPS) from gram-negative bacteria. The PBMCs were then incubated at 37° C. and 5% CO₂ for a further 6 hours and the cell supernatant was collected and a Homogenous Time Resolved Fluorescence (HTRF) assay was carried out following the manufacturer's instructions. Cytokine release from the PBMCs was captured using a Perkin Elmer ENVISION 2104 microplate reader set at 615 nm and 665 nm respectively. Analysis of cytotoxicity under similar conditions using 100,000 PBMCs in a 96-well plate dosed with the same test compounds, with or without PMA and ionomycin stimulation at the 2 hour time point, revealed that any minor cell loss that had occurred, was insufficient to account for the inhibition of cytokine release observed in each of the six experiments.

TABLE 4
Inhibitory activity of compounds of the invention
against a range of cytokines.

| | % Inhibition | | | | | | | | |
|---|---|---|---|---|---|---|---|---|---|
| | TNF-α | | | IFN-γ | | | IL-6 | | |
| | 10 | | 0.1 | 10 | | 0.1 | 10 | | 0.1 |
| Compound | μM | 1 μM | μM | μM | 1 μM | μM | μM | 1 μM | μM |
| 3507 | 90 | 68 | 52 | 77 | 42 | 16 | 68 | 21 | −9 |
| 3520 | −14 | −10 | −8 | 4 | 0 | 1 | 3 | −9 | −10 |
| 3534 | −12 | 5 | 2 | 85 | 39 | 23 | 48 | 21 | 13 |
| 3538 | −7 | −4 | −10 | 86 | 58 | 24 | −10 | −13 | −13 |

TABLE 5
Inhibitory activity of compounds of the invention
against a range of cytokines.

| | % Inhibition | | | | | | | | |
|---|---|---|---|---|---|---|---|---|---|
| | IL-21 | | | IL-17A | | | IL-23 | | |
| | 10 | | 0.1 | 10 | | 0.1 | 10 | | |
| Compound | μM | 1 μM | μM | μM | 1 μM | μM | μM | 1 μM | 0.1 μM |
| 3507 | 19 | 5 | −6 | 13 | 4 | 2 | 20 | 0 | −7 |
| 3520 | −12 | −13 | −8 | 1 | −7 | −11 | 12 | −1 | −8 |
| 3534 | 11 | −8 | 0 | 12 | 1 | 1 | 20 | −1 | −5 |
| 3538 | 11 | 8 | 1 | 86 | 42 | 21 | 30 | −20 | −19 |

### Tolerance and In Vivo Efficacy of Compound 3507

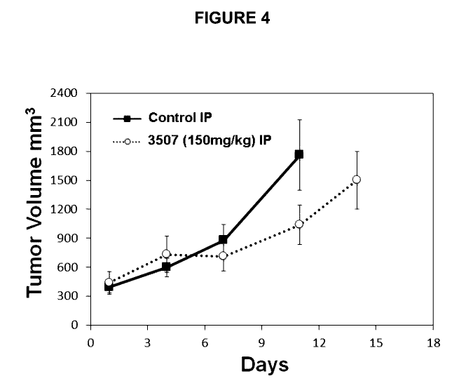

The in vivo efficacy of compound 3507 was evaluated in the CHLA20 neuroblastoma xenograft model. CHLA20 tumours were established in athymic nude mice by injecting 1.0×10⁷ tumour cells subcutaneously in the right flank. Dosing of animals commenced when tumour volume reached ˜200-400 mm³. Animals (˜n=5+) were randomly divided into treatment and control groups. Compound 3507 was dosed daily by intraperotineal (IP) injection at 150 mg/kg in 30% w/v Captisol (a cyclodextrin-containing formulation). After 18 days of treatment, compound 3507 was found to be well tolerated and significantly slowed tumour growth compared to vehicle control (FIG. 4).

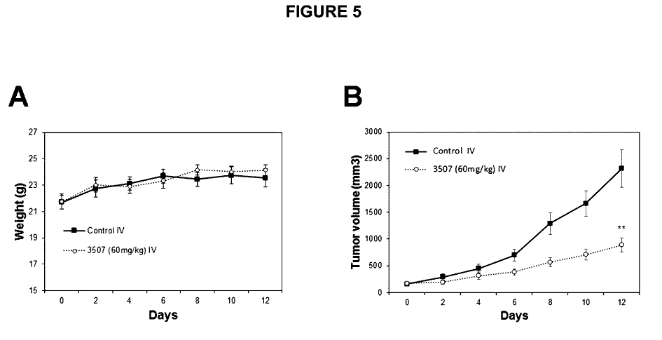

The in vivo efficacy of 3507 was also evaluated in a human melanoma (A375) xenograft model. A375 tumours were established in female Foxn-1 nu/nu athymic mice by injecting approximately five million cells subcutaneously in the right flank region of the animal. When the tumours reached 130-150 mm³ the animals were randomized into four groups, (n=8 or 12 animals/group) so that the average tumour volume of all the groups was same. Group 1 received vehicle (30% w/v Dexolve-7 in sterile water) twice a week intravenously, and Group 2 was dosed with 3507/Dexolve-7 at 60 mg/kg, twice a week intravenously. Tumours and body weight was measured two to three times in a week. In addition, throughout the study period mice were monitored daily for clinical condition. Body weights of the animals treated with compound 3507 were comparable to the control group throughout the study period demonstrating that compound 3507 was well tolerated (FIG. 5A). In line with the neuroblastoma study, after 14 days treatment compound 3507 was found to significantly reduced melanoma tumour growth by ˜60% compared to vehicle control (FIG. 5B).

### Reference Articles

Broschat, K. O. (1990). Tropomyosin prevents depolymerisation of actin filaments from the pointed end. J Biol Chem 265, 21323-21329.
Kostyukova, A. S., and Hitchcock-DeGregori, S. E. (2004). Effect of the structure of the N terminus of tropomyosin on tropomodulin function. J Biol Chem 279, 5066-5071.
Pollard, T. D. (1986). Rate constants for the reactions of ATP- and ADP-actin with the ends of actin filaments. J Cell Biol 103, 2747-2754.
Bonello, T. B (2013). Characterising the impact of tropomyosin targeting compounds in the actin cytoskeleton. Ph.D thesis, School of Medical Sciences, University of New South Wales, Australia
Vindin, H., Bischof, L., Gunning, P. & Stehn, J. Validation of an algorithm to quantify changes in actin cytoskeletal organization. J Biomol Screen 19, 354-368 (2014).

It will be understood that the invention disclosed and defined in this specification extends to all alternative combinations of two or more of the individual features mentioned or evident from the text or drawings. All of these different combinations constitute various alternative aspects of the invention.

1. A compound of formula (I) or a pharmaceutically acceptable drug or prodrug thereof: 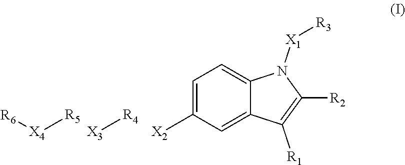
wherein:
R₁ and R₂ are independently H or C₁-C₆ alkyl;
R₃ is N(R₇)₂ or a 3- to 7-membered carbocyclic ring wherein between 1 and 3 ring carbon atoms may optionally be replaced by S, N, O, NH or NR₇ and wherein the ring may optionally be substituted by R₇;
R₄ and R₅ are independently 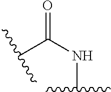 [SMILES: *NC(*)=O]
or a 5- or 6-membered carbocyclic ring wherein between 1 and 3 ring carbon atoms may optionally be replaced with S, N, O, NH or NR₈ and wherein the ring may optionally be substituted by R₈;
R₆ is a C₁-C₆ alkyl group, a C₂-C₆ alkene group, or a monocyclic or bicyclic carbocyclic ring having between 5 and 10 ring carbon atoms wherein 1 or 2 ring carbon atoms may optionally be replaced with S, O, N, NH or NR₇ and wherein the ring may optionally be substituted with R₈, or R₆ is 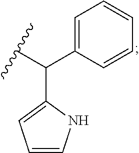 [SMILES: *C(c1ccccc1)c1ccc[nH]1]
X₁ is an alkyl group having between 1 and 10 carbon atoms, or an alkene group having between 2 and 10 carbon atoms;
X₂, X₃ and X₄ are independently absent or selected from the group consisting of: S, O, NH, NH(R₇), C(O), C(O)NH, an alkyl group having between 1 and 10 carbon atoms, an alkene group having between 2 and 10 carbon atoms, CH(R₇)CHC(R₇)C(O), (CH₂)₀₋₅C(R₇)C(R₇)(CH₂)₀₋₅, and a 5- or 6-membered carbocyclic ring wherein between 1 and 3 ring carbon atoms may optionally be replaced by S, N, O, NH or NR₇;
X₅ is O, NH, NR₇ or S;
R₇ is H, C₁-C₆ alkyl, (CH₂)₁₋₅OMe, CF₃, CN or OCF₃; and
R₈ is H, OH, alkyl, alkenyl, halo, alkoxy, amino, alkylamino, dialkylamino or a dioxolane ring fused to 2 adjacent carbon atoms of R₄, R₅ or R₆.

2. A compound according to claim 1, wherein X₁ is an alkyl group having between 1 and 10 carbon atoms.

3. A compound according to claim 1 or 2, wherein R₃ is N(R₇)₂ or a 4-, 5-, 6- or 7-membered carbocyclic ring wherein between 1 and 3 ring carbon atoms may optionally be replaced by S, N, O, NH or NR₇ and wherein the ring may optionally be substituted by R₇.

4. A compound according to claim 3, wherein the carbocyclic ring is a cycloalkyl group.

5. A compound according to any one of the preceding claims, wherein R₁ and R₂ are independently C₁-C₆ alkyl.

6. A compound according to any one of the preceding claims, wherein X₂, X₃ and X₄ are independently selected from the group consisting of: S, O, NH, NHR₇, C(O), C(O)NH, an alkyl group having between 1 and 10 carbon atoms, CH(R₇)CHC(R₇)C(O), (CH₂)₀₋₅C(R₇)C(R₇)(CH₂)₀₋₅, and a 5-membered carbocyclic ring wherein between 1 and 3 ring carbon atoms may optionally be replaced by S, N, O, NH or NR₇.

7. A compound according to claim 6, wherein the alkyl group has between 1 and 5 carbon atoms.

8. A compound according to claim 6, wherein the carbocyclic ring is an aryl group.

9. A compound according to claim 6 or 8, wherein, in the carbocyclic ring, 1 or 2 ring carbon atoms may optionally be replaced by S, N, O, NH or NR₇.

10. A compound according to any one of the preceding claims, wherein R₄ and R₅ are independently a 5- or 6-membered aryl or cycloalkyl group wherein between 1 and 3 ring carbon atoms may optionally be replaced with S, N, O, NH or NR₈ and wherein the ring may optionally be substituted by R₈.

11. A compound according to any one of the preceding claims, wherein R₆ is a C₁-C₆ alkyl group or a monocyclic or bicyclic aryl group having between 6 and 10 ring carbon atoms wherein 1 or 2 ring carbon atoms may optionally be replaced with S, O, N, NH or NR₇ and wherein the ring may optionally be substituted with R₈, or R₆ is:

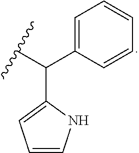 [SMILES: *C(c1ccccc1)c1ccc[nH]1]

12. A compound according to claim 11, wherein the C₁-C₆ alkyl group is CH₃ or CH₂CH₃.

13. A compound according to any one of the preceding claims, wherein the compound of formula (I), or a pharmaceutically acceptable drug or prodrug thereof, is: 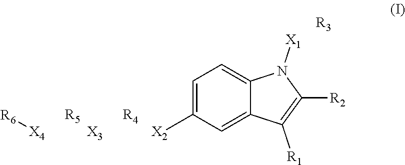
wherein:

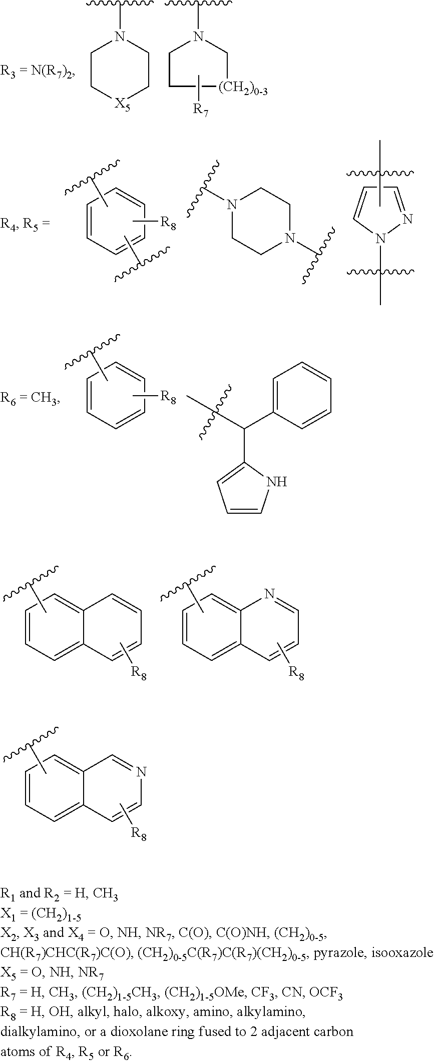

14. A compound according to any one of the preceding claims, wherein R₁ and R₂ are both CH₃ or CH₂CH₃.

15. A compound according to any one of the preceding claims, wherein X₁ is an alkyl group having between 1 and 5 carbon atoms.

16. A compound according to claim 15, wherein X₁ is CH₂, (CH₂)₂ or (CH₂)₃.

17. A compound according to any one of the preceding claims, wherein R₃ is a 4-, 5-, or 6-membered cycloalkyl group.

18. A compound according to claim 17, wherein R₃ is:

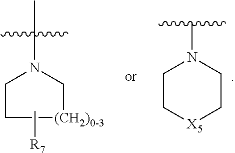

19. A compound according to claim 17, wherein R₃ is a 6-membered cycloalkyl group.

20. A compound according to claim 19, wherein R₃ is:

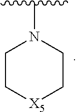

21. A compound according to any one of the preceding claims, wherein X₅ is NH or NR₇.

22. A compound according to claim 21, wherein R₇ is C₁-C₆ alkyl.

23. A compound according to claim 22, wherein R₇ is CH₃ or CH₂CH₃.

24. A compound according to any one of the preceding claims, wherein X₂ is an alkyl group having between 1 and 10 carbon atoms, O or NH.

25. A compound according to claim 24, wherein X₂ is (CH₂)₁₋₅.

26. A compound according to claim 25, wherein X₂ is CH₂, (CH₂)₂ or (CH₂)₃.

27. A compound according to any one of the preceding claims, wherein R₄ is a 5- or 6-membered aryl group wherein between 1 and 3 ring carbon atoms may optionally be replaced with S, N, O, NH or NR₈ and wherein the ring may optionally be substituted by R₈.

28. A compound according to claim 27, wherein R₄ is:

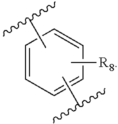

29. A compound according to claim 28, wherein R₈ is H.

30. A compound according to any one of the preceding claims, wherein X₃ is C(O).

31. A compound according to any one of the preceding claims, wherein R₅ is a 5- or 6-membered cycloalkyl group wherein between 1 and 3 ring carbon atoms may optionally be replaced with S, N, O, NH or NR₈ and wherein the ring may optionally be substituted by R₈.

32. A compound according to claim 31, wherein R₅ is:

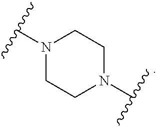 [SMILES: *N1CCN(*)CC1]

33. A compound according to any one of the preceding claims, wherein X₄ is an alkyl group having between 1 and 5 carbon atoms.

34. A compound according to claim 33, wherein X₄ is CH₂, (CH₂)₂ or (CH₂)₃.

35. A compound according to any one of the preceding claims, wherein R₆ is a bicyclic aryl group having 9 or 10 ring carbon atoms wherein 1 or 2 ring carbon atoms may optionally be replaced with S, O, N, NH or NR₇ and wherein the ring may optionally be substituted with R₈.

36. A compound according to claim 35, wherein R₆ is selected from:

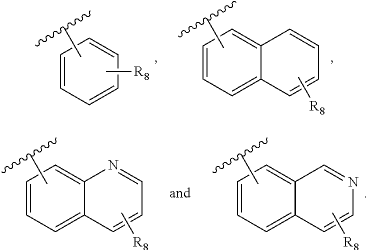

37. A compound according to claim 35 or 36, wherein R₈ is selected from H, alkoxy, halo and a dioxalane ring fused to two adjacent carbon atoms of R₆.

38. A compound according to claim 37, wherein R₈ is alkoxy.

39. A compound according to claim 38, wherein R₈ is OCH₃ or OCH₂CH₃.

40. A compound according to claim 37, wherein R₈ is halo.

41. A compound according to claim 40, wherein R₈ is fluorine.

42. A compound according to any one of the preceding claims, wherein the compound is selected from the group consisting of:

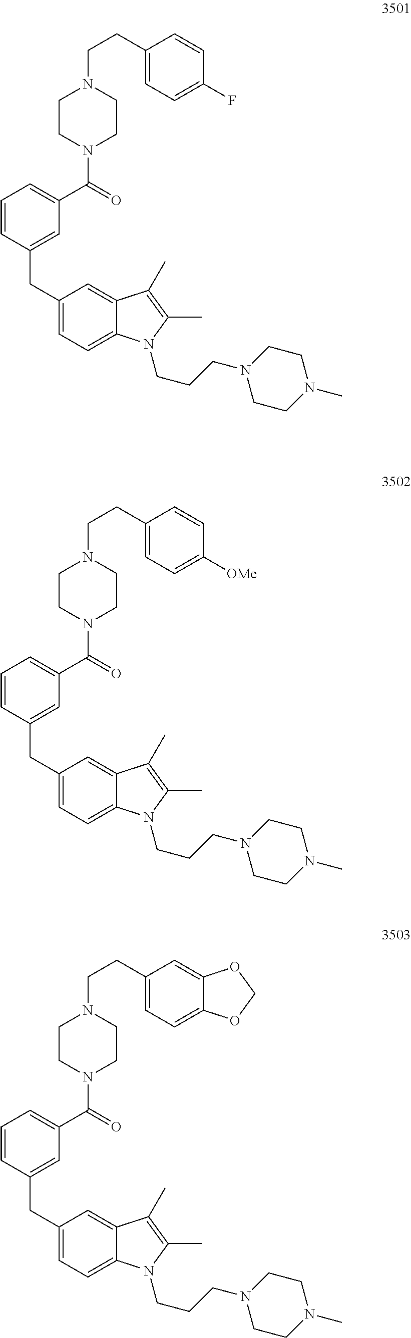 [SMILES: Cc1c(C)n(CCCN2CCN(C)CC2)c2ccc(Cc3cccc(C(=O)N4CCN(CCc5ccc(F)cc5)CC4)c3)cc12 ; COc1ccc(CCN2CCN(C(=O)c3cccc(Cc4ccc5c(c4)c(C)c(C)n5CCCN4CCN(C)CC4)c3)CC2)cc1 ; Cc1c(C)n(CCCN2CCN(C)CC2)c2ccc(Cc3cccc(C(=O)N4CCN(CCc5ccc6c(c5)OCO6)CC4)c3)cc12]

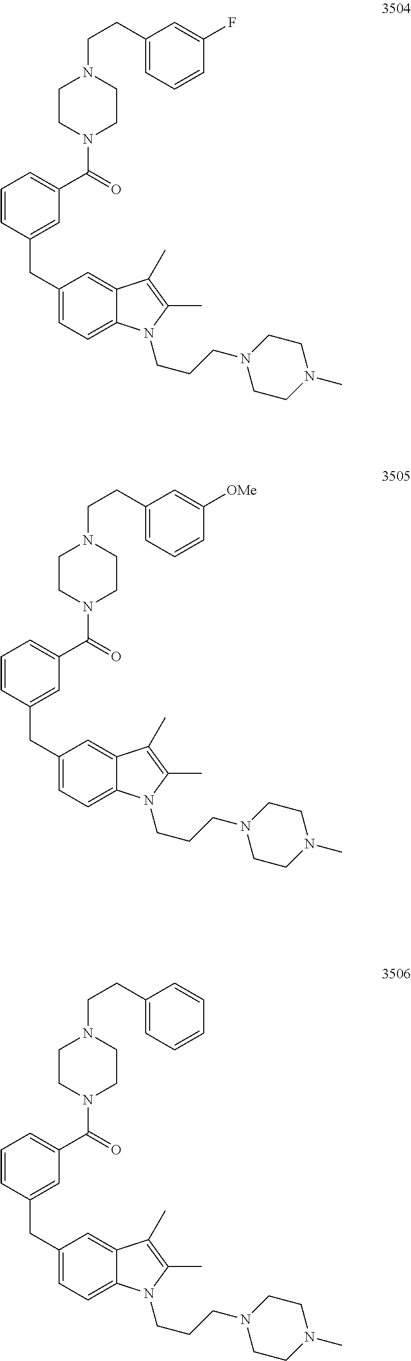 [SMILES: Cc1c(C)n(CCCN2CCN(C)CC2)c2ccc(Cc3cccc(C(=O)N4CCN(CCc5cccc(F)c5)CC4)c3)cc12 ; COc1cccc(CCN2CCN(C(=O)c3cccc(Cc4ccc5c(c4)c(C)c(C)n5CCCN4CCN(C)CC4)c3)CC2)c1 ; Cc1c(C)n(CCCN2CCN(C)CC2)c2ccc(Cc3cccc(C(=O)N4CCN(CCc5ccccc5)CC4)c3)cc12]

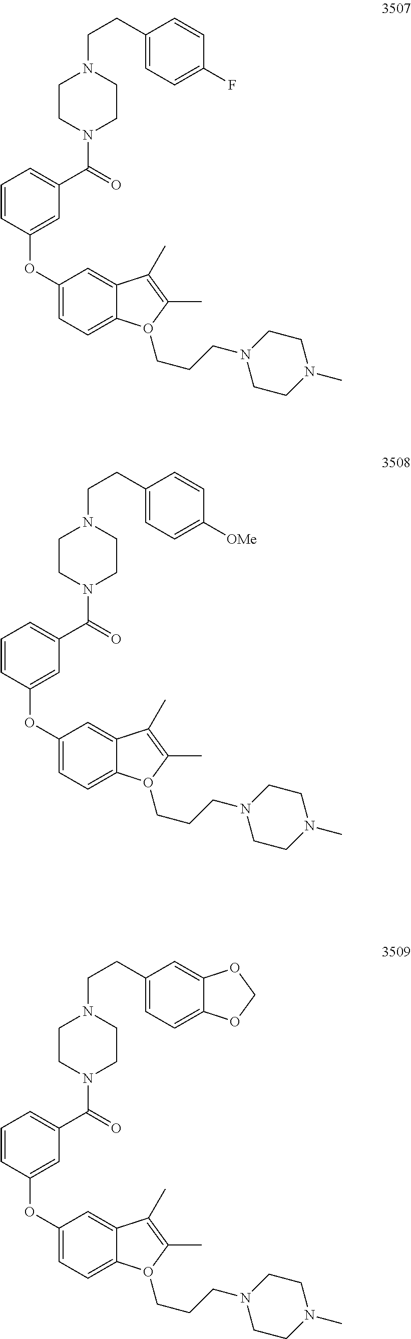

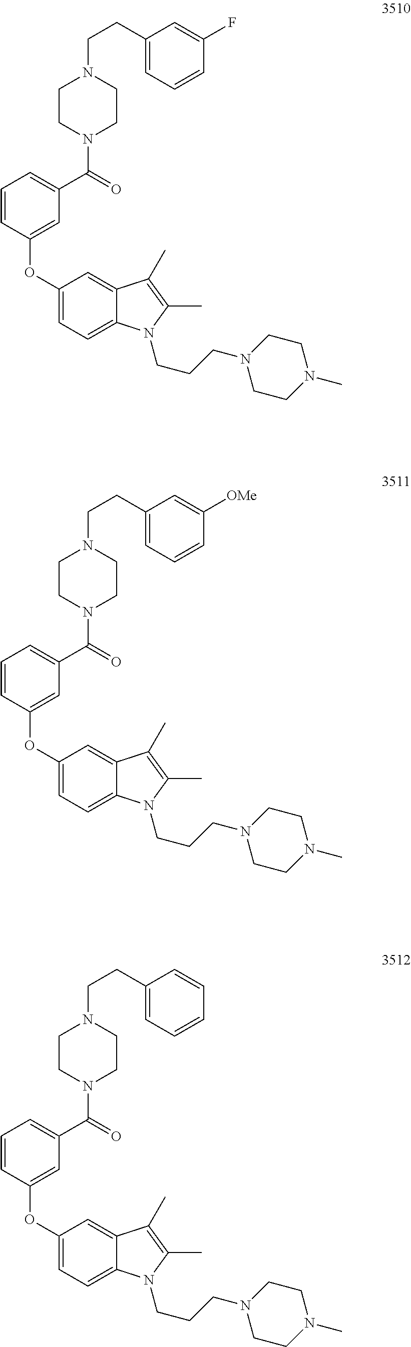 [SMILES: Cc1c(C)n(CCCN2CCN(C)CC2)c2ccc(Oc3cccc(C(=O)N4CCN(CCc5cccc(F)c5)CC4)c3)cc12 ; COc1cccc(CCN2CCN(C(=O)c3cccc(Oc4ccc5c(c4)c(C)c(C)n5CCCN4CCN(C)CC4)c3)CC2)c1 ; Cc1c(C)n(CCCN2CCN(C)CC2)c2ccc(Oc3cccc(C(=O)N4CCN(CCc5ccccc5)CC4)c3)cc12]

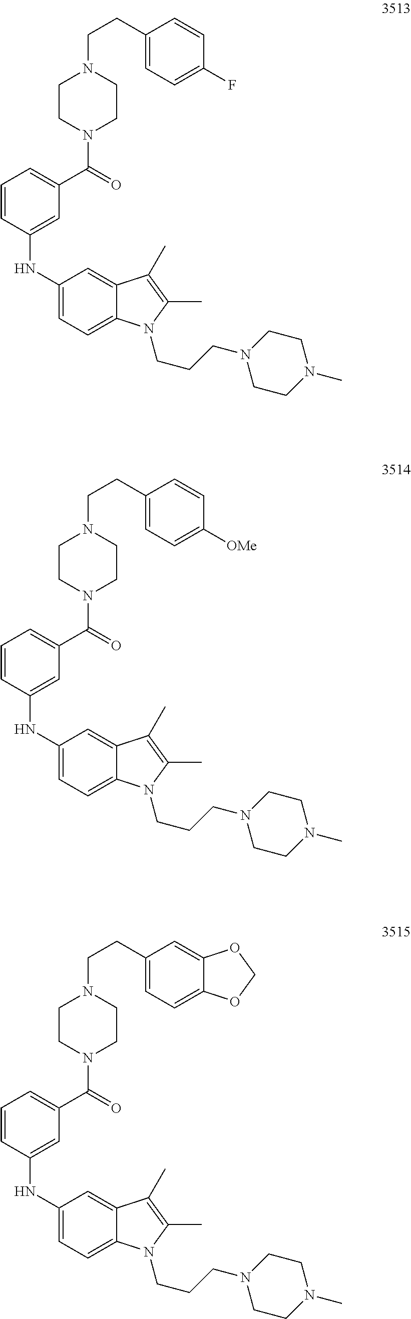 [SMILES: Cc1c(C)n(CCCN2CCN(C)CC2)c2ccc(Nc3cccc(C(=O)N4CCN(CCc5ccc(F)cc5)CC4)c3)cc12 ; COc1ccc(CCN2CCN(C(=O)c3cccc(Nc4ccc5c(c4)c(C)c(C)n5CCCN4CCN(C)CC4)c3)CC2)cc1 ; Cc1c(C)n(CCCN2CCN(C)CC2)c2ccc(Nc3cccc(C(=O)N4CCN(CCc5ccc6c(c5)OCO6)CC4)c3)cc12]

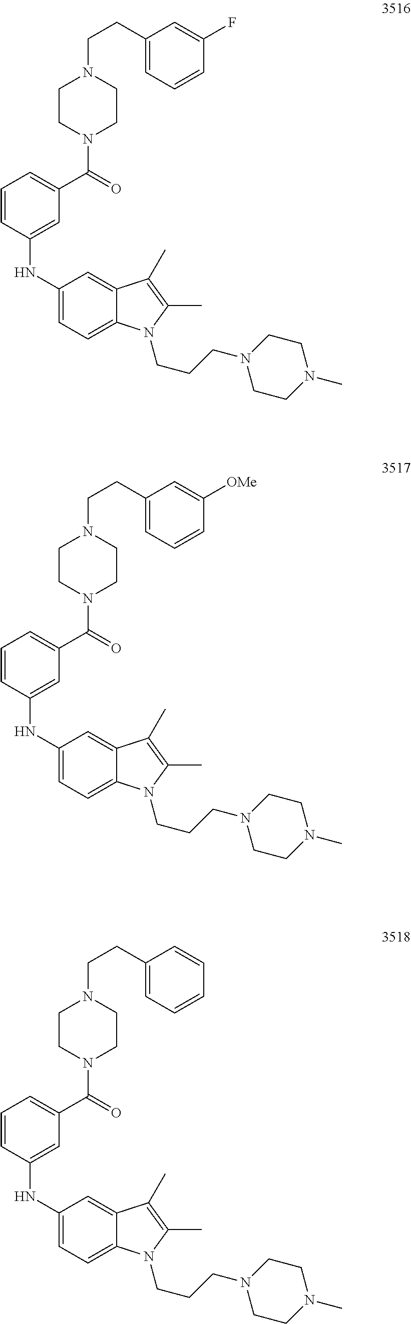 [SMILES: Cc1c(C)n(CCCN2CCN(C)CC2)c2ccc(Nc3cccc(C(=O)N4CCN(CCc5cccc(F)c5)CC4)c3)cc12 ; COc1cccc(CCN2CCN(C(=O)c3cccc(Nc4ccc5c(c4)c(C)c(C)n5CCCN4CCN(C)CC4)c3)CC2)c1 ; Cc1c(C)n(CCCN2CCN(C)CC2)c2ccc(Nc3cccc(C(=O)N4CCN(CCc5ccccc5)CC4)c3)cc12]

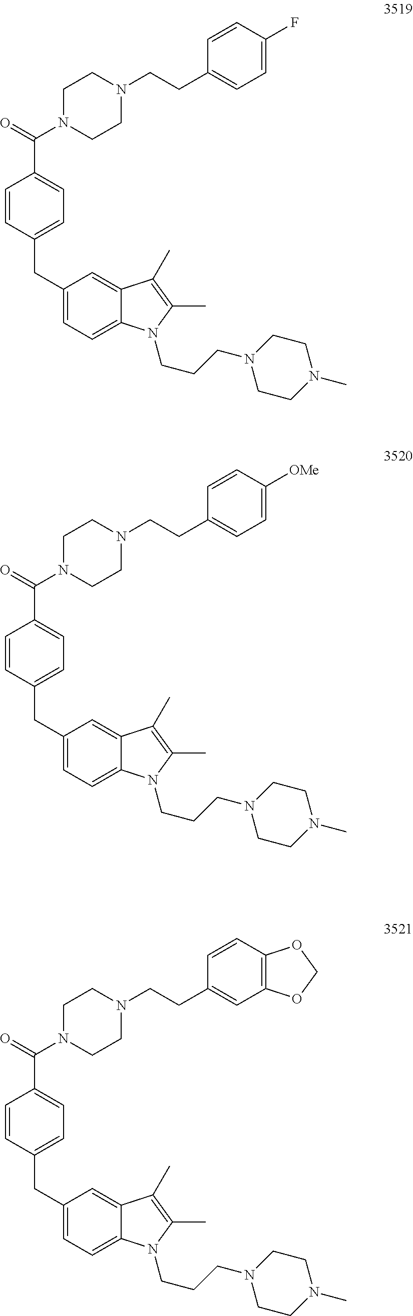 [SMILES: Cc1c(C)n(CCCN2CCN(C)CC2)c2ccc(Cc3ccc(C(=O)N4CCN(CCc5ccc(F)cc5)CC4)cc3)cc12 ; COc1ccc(CCN2CCN(C(=O)c3ccc(Cc4ccc5c(c4)c(C)c(C)n5CCCN4CCN(C)CC4)cc3)CC2)cc1 ; Cc1c(C)n(CCCN2CCN(C)CC2)c2ccc(Cc3ccc(C(=O)N4CCN(CCc5ccc6c(c5)OCO6)CC4)cc3)cc12]

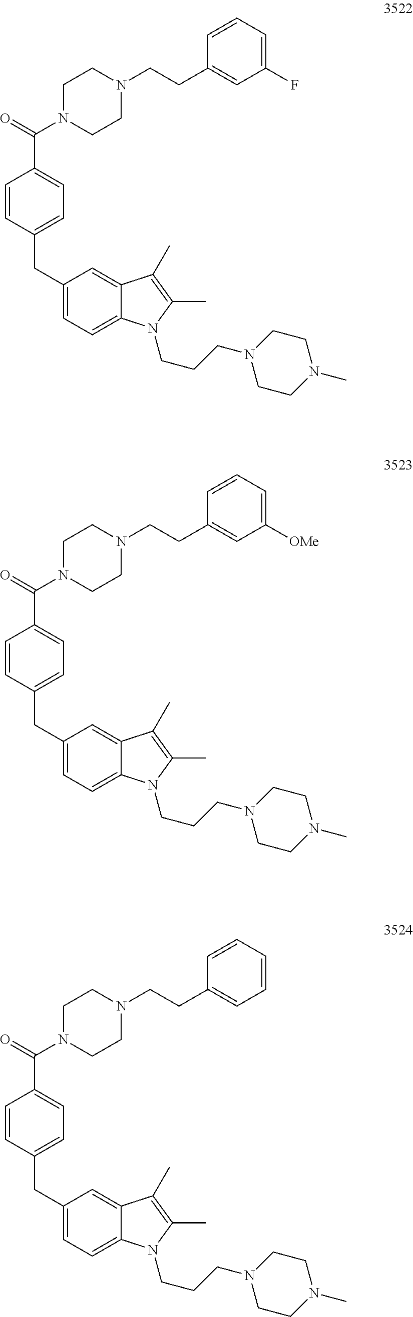 [SMILES: Cc1c(C)n(CCCN2CCN(C)CC2)c2ccc(Cc3ccc(C(=O)N4CCN(CCc5cccc(F)c5)CC4)cc3)cc12 ; COc1cccc(CCN2CCN(C(=O)c3ccc(Cc4ccc5c(c4)c(C)c(C)n5CCCN4CCN(C)CC4)cc3)CC2)c1 ; Cc1c(C)n(CCCN2CCN(C)CC2)c2ccc(Cc3ccc(C(=O)N4CCN(CCc5ccccc5)CC4)cc3)cc12]

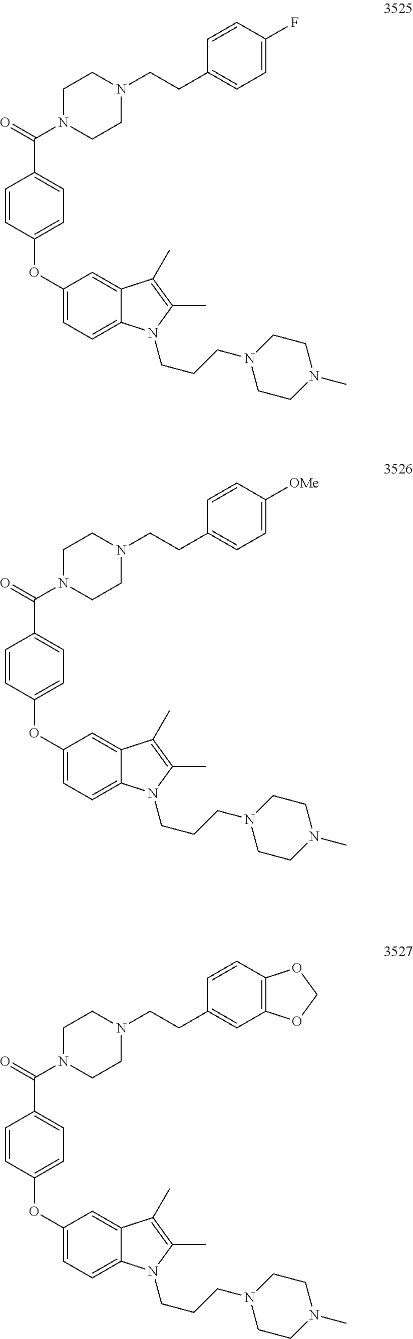 [SMILES: Cc1c(C)n(CCCN2CCN(C)CC2)c2ccc(Oc3ccc(C(=O)N4CCN(CCc5ccc(F)cc5)CC4)cc3)cc12 ; COc1ccc(CCN2CCN(C(=O)c3ccc(Oc4ccc5c(c4)c(C)c(C)n5CCCN4CCN(C)CC4)cc3)CC2)cc1 ; Cc1c(C)n(CCCN2CCN(C)CC2)c2ccc(Oc3ccc(C(=O)N4CCN(CCc5ccc6c(c5)OCO6)CC4)cc3)cc12]

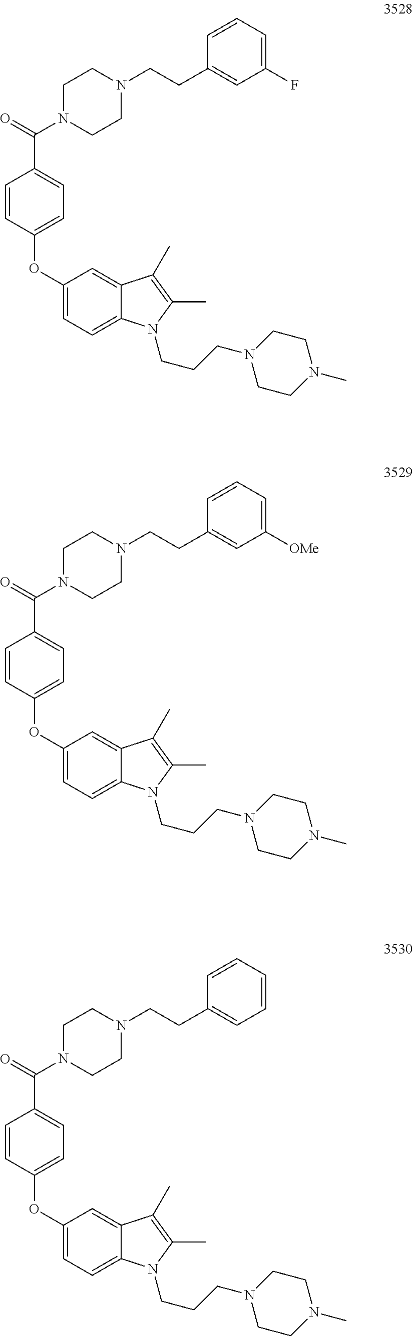 [SMILES: Cc1c(C)n(CCCN2CCN(C)CC2)c2ccc(Oc3ccc(C(=O)N4CCN(CCc5cccc(F)c5)CC4)cc3)cc12 ; COc1cccc(CCN2CCN(C(=O)c3ccc(Oc4ccc5c(c4)c(C)c(C)n5CCCN4CCN(C)CC4)cc3)CC2)c1 ; Cc1c(C)n(CCCN2CCN(C)CC2)c2ccc(Oc3ccc(C(=O)N4CCN(CCc5ccccc5)CC4)cc3)cc12]

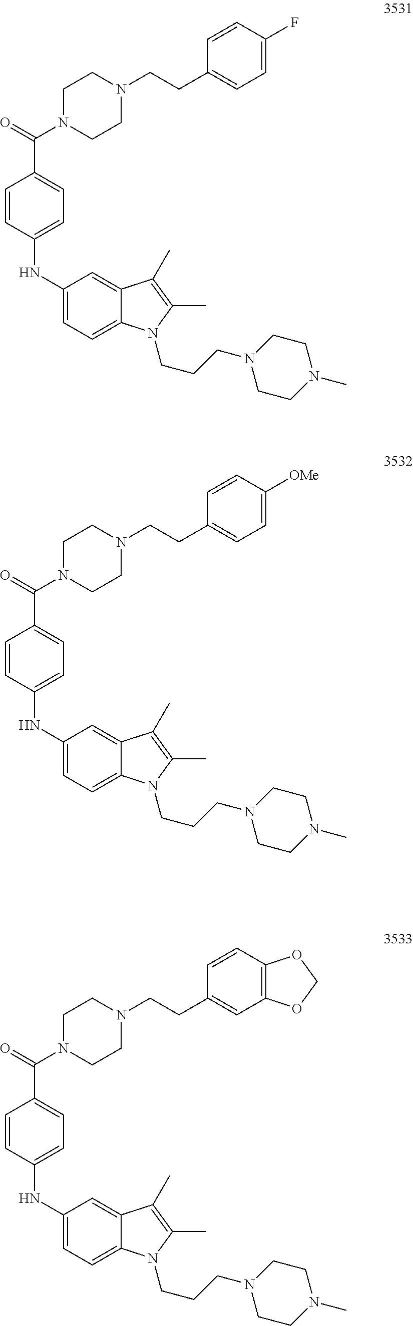 [SMILES: Cc1c(C)n(CCCN2CCN(C)CC2)c2ccc(Nc3ccc(C(=O)N4CCN(CCc5ccc(F)cc5)CC4)cc3)cc12 ; COc1ccc(CCN2CCN(C(=O)c3ccc(Nc4ccc5c(c4)c(C)c(C)n5CCCN4CCN(C)CC4)cc3)CC2)cc1 ; Cc1c(C)n(CCCN2CCN(C)CC2)c2ccc(Nc3ccc(C(=O)N4CCN(CCc5ccc6c(c5)OCO6)CC4)cc3)cc12]

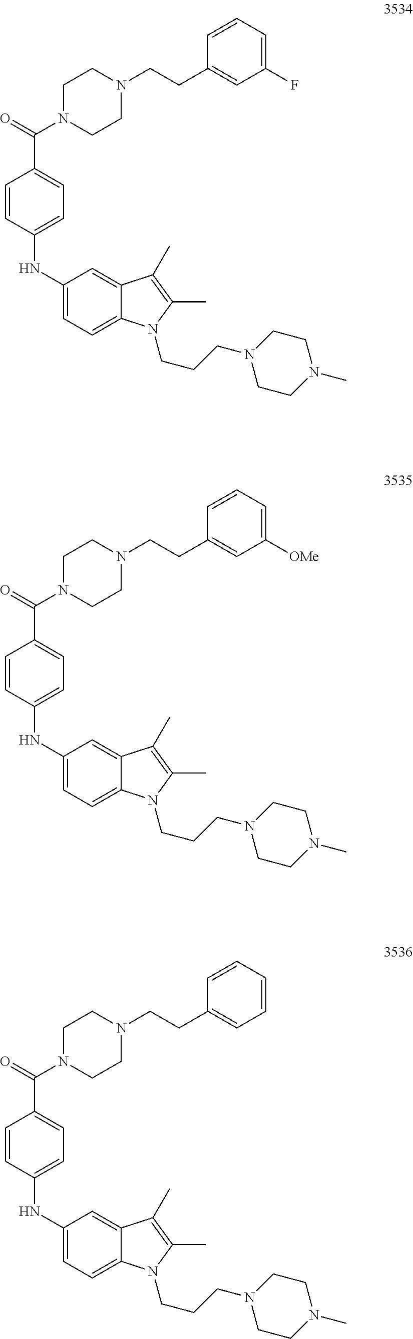 [SMILES: Cc1c(C)n(CCCN2CCN(C)CC2)c2ccc(Nc3ccc(C(=O)N4CCN(CCc5cccc(F)c5)CC4)cc3)cc12 ; COc1cccc(CCN2CCN(C(=O)c3ccc(Nc4ccc5c(c4)c(C)c(C)n5CCCN4CCN(C)CC4)cc3)CC2)c1 ; Cc1c(C)n(CCCN2CCN(C)CC2)c2ccc(Nc3ccc(C(=O)N4CCN(CCc5ccccc5)CC4)cc3)cc12]

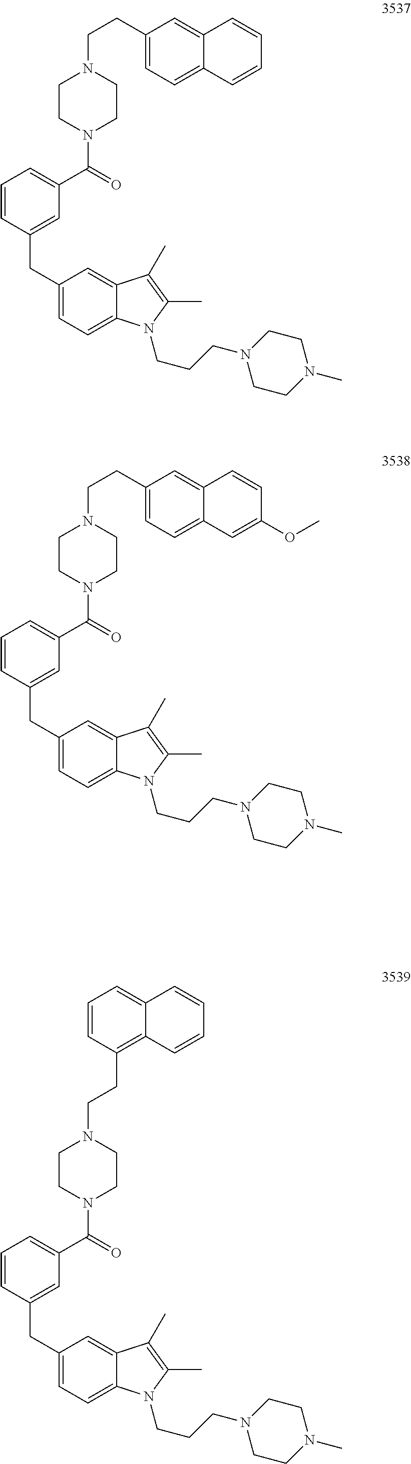 [SMILES: Cc1c(C)n(CCCN2CCN(C)CC2)c2ccc(Cc3cccc(C(=O)N4CCN(CCc5ccc6ccccc6c5)CC4)c3)cc12 ; COc1ccc2cc(CCN3CCN(C(=O)c4cccc(Cc5ccc6c(c5)c(C)c(C)n6CCCN5CCN(C)CC5)c4)CC3)ccc2c1 ; Cc1c(C)n(CCCN2CCN(C)CC2)c2ccc(Cc3cccc(C(=O)N4CCN(CCc5cccc6ccccc56)CC4)c3)cc12]

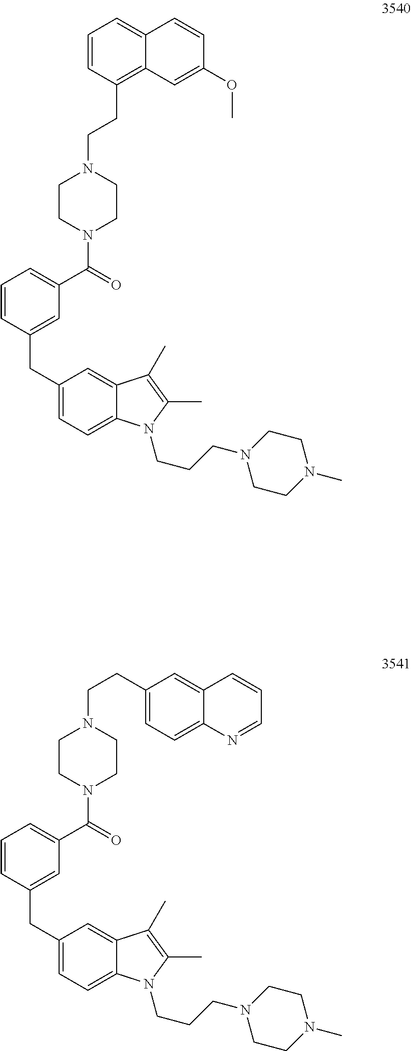 [SMILES: COc1ccc2cccc(CCN3CCN(C(=O)c4cccc(Cc5ccc6c(c5)c(C)c(C)n6CCCN5CCN(C)CC5)c4)CC3)c2c1 ; Cc1c(C)n(CCCN2CCN(C)CC2)c2ccc(Cc3cccc(C(=O)N4CCN(CCc5ccc6ncccc6c5)CC4)c3)cc12]

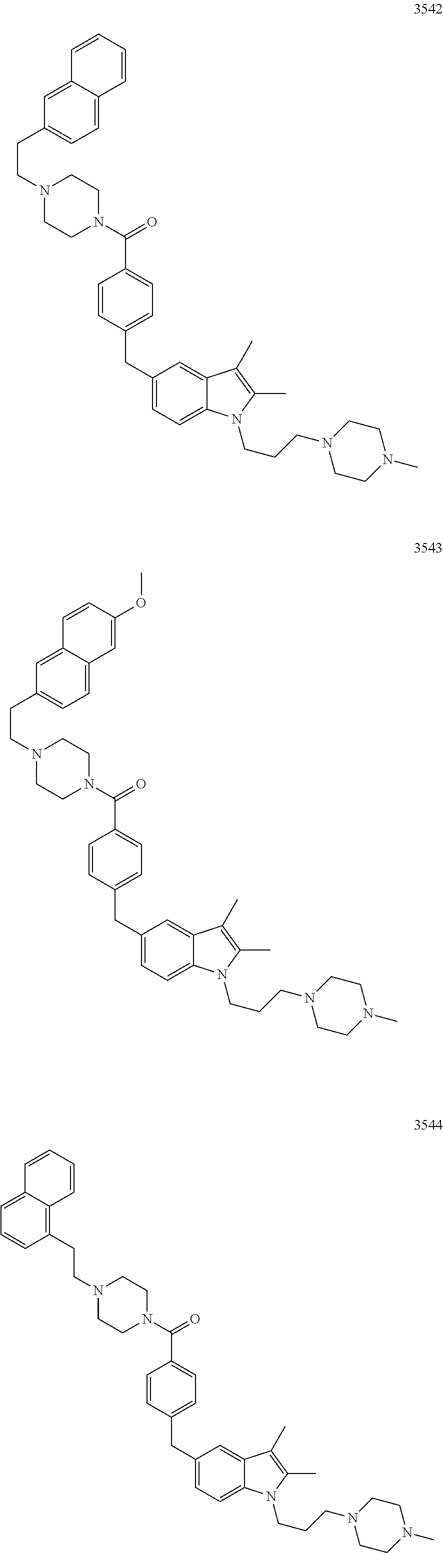 [SMILES: Cc1c(C)n(CCCN2CCN(C)CC2)c2ccc(Cc3ccc(C(=O)N4CCN(CCc5ccc6ccccc6c5)CC4)cc3)cc12 ; COc1ccc2cc(CCN3CCN(C(=O)c4ccc(Cc5ccc6c(c5)c(C)c(C)n6CCCN5CCN(C)CC5)cc4)CC3)ccc2c1 ; Cc1c(C)n(CCCN2CCN(C)CC2)c2ccc(Cc3ccc(C(=O)N4CCN(CCc5cccc6ccccc56)CC4)cc3)cc12]

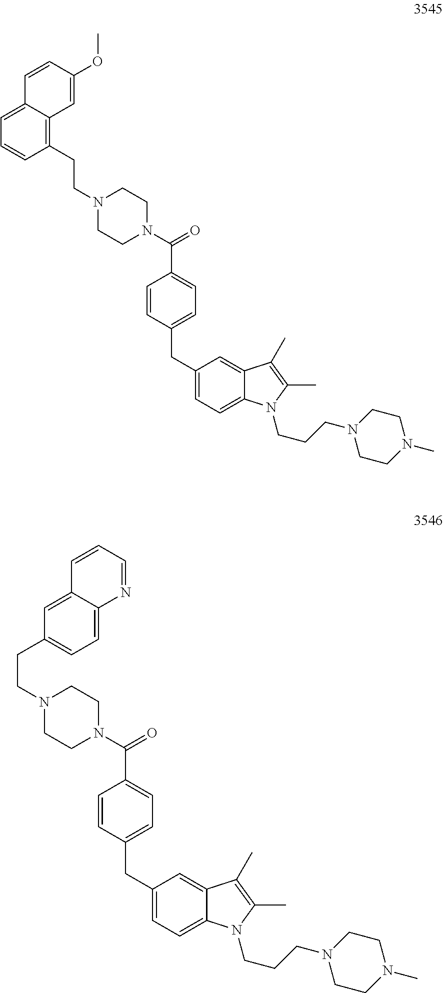 [SMILES: COc1ccc2cccc(CCN3CCN(C(=O)c4ccc(Cc5ccc6c(c5)c(C)c(C)n6CCCN5CCN(C)CC5)cc4)CC3)c2c1 ; Cc1c(C)n(CCCN2CCN(C)CC2)c2ccc(Cc3ccc(C(=O)N4CCN(CCc5ccc6ncccc6c5)CC4)cc3)cc12]

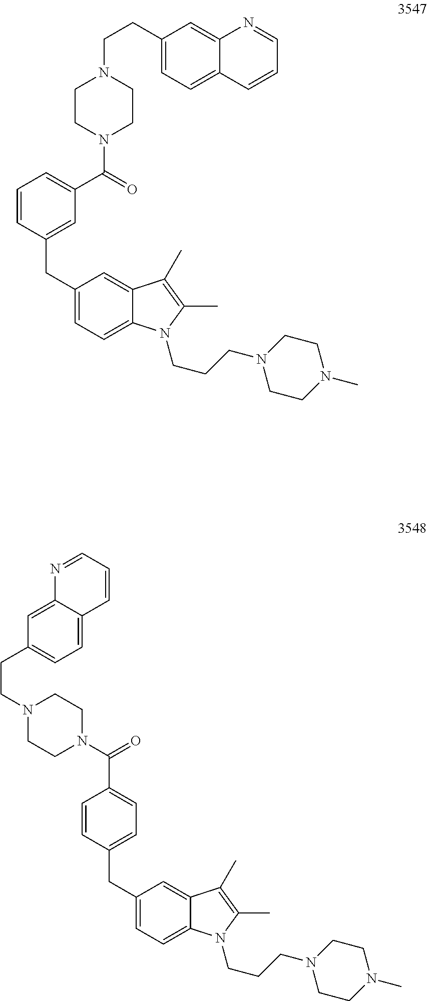 [SMILES: Cc1c(C)n(CCCN2CCN(C)CC2)c2ccc(Cc3cccc(C(=O)N4CCN(CCc5ccc6cccnc6c5)CC4)c3)cc12 ; Cc1c(C)n(CCCN2CCN(C)CC2)c2ccc(Cc3ccc(C(=O)N4CCN(CCc5ccc6cccnc6c5)CC4)cc3)cc12]

43. A pharmaceutical composition for the treatment or prevention of a proliferative disease wherein the composition includes a compound according to any one of claims 1 to 42.

44. A method of treating or preventing a proliferative disease including administering to a subject a therapeutically effective amount of a compound according to any one of claims 1 to 42.

45. Use of a compound according to any one of claims 1 to 42 for the treatment or prevention of a proliferative disease.

46. Use of a compound according to any one of claims 1 to 42 or the pharmaceutical composition of claim 43 in the manufacture of a medicament for treating or preventing a proliferative disease.

47. A pharmaceutical composition according to claim 43, a method according to claim 44, or a use according to claim 45 or 46, wherein the proliferative disease is cancer.

48. A pharmaceutical composition for preventing the recurrence of a solid tumour wherein the composition includes a compound according to any one of claims 1 to 42.

49. A method of preventing the recurrence of a solid tumour including administering to a subject a therapeutically effective amount of a compound according to any one of claims 1 to 42.

50. Use of a compound according to any one of claims 1 to 42 for preventing the recurrence of a solid tumour.

51. Use of a compound according to any one of claims 1 to 42 or the pharmaceutical composition of claim 48 in the manufacture of a medicament for preventing the recurrence of a solid tumour.

52. A pharmaceutical composition for the treatment of an inflammatory disease or disorder wherein the composition includes a compound according to any one of claims 1 to 42.

53. A method of treating an inflammatory disease or disorder including administering to a subject a therapeutically effective amount of a compound according to any one of claims 1 to 42.

54. Use of a compound according to any one of claims 1 to 42 for treatment of an inflammatory disease or disorder.

55. Use of a compound according to any one of claims 1 to 42 or the pharmaceutical composition of claim 52 in the manufacture of a medicament for treating an inflammatory disease or disorder.

56. A pharmaceutical composition according to claim 52, a method according to claim 53 or a use according to claim 54 or 55, wherein the inflammatory disease or disorder is selected from osteoarthritis, inflammatory bowel disease, ulcerative proctitis, distal colitis, autoimmune disorders, asthma and diseases involving pulmonary inflammation, and cardiovascular disorders.
